(19) United States
(12) Patent Application Publication
(10) Pub. No.: US 2025/0302394 A1
(43) Pub. Date: Oct. 2, 2025

(54) QUANTITATIVE MAGNETIC RESONANCE IMAGING AND TUMOR FORECASTING

(71) Applicant: BOARD OF REGENTS, THE UNIVERSITY OF TEXAS SYSTEM, Austin, TX (US)

(72) Inventors: Thomas YANKEELOV, Austin, TX (US); Angela JARRETT, Austin, TX (US); David Andrew HORMUTH, II, Austin, TX (US); Anum KAZEROUNI, Austin, TX (US); Chengyue WU, Austin, TX (US); Stephanie BARNES, Austin, TX (US)

(21) Appl. No.: 18/694,076

(22) PCT Filed: Sep. 21, 2022
(86) PCT No.: PCT/US2022/044285

§ 371 Date: Mar. 21, 2024

Related U.S. Application Data
(60) Provisional application No. 63/247,233, filed on Sep. 22, 2021.
(60) Provisional application No. 63/257,740, filed on Oct. 20, 2021.

(51) Int. Cl. A61B 5/00; G06T 7/00; G16H 20/10; G16H 50/30

(52) U.S. Cl. CPC A61B 5/7275; A61B 5/4848; A61B 5/7267; A61B 5/7425; G06T 7/0016; G16H 20/10; G16H 50/30; G06T 2207/10096; G06T 2207/30096

(57) ABSTRACT

Disclosed are approaches to data acquisition, analysis, and computational forecasting that employs quantitative MRI data to predict the response of cancer to therapy. Example protocols detail how to acquire needed images followed by registration, segmentation, quantitative perfusion and diffusion analysis, model calibration, and prediction. The response of individual cancer patients to therapy is forecast by application of a biophysical, reaction-diffusion model to these data. Application of the protocol results in coregistered MRI data from at least two scan visits that quantifies an individual tumor's size, cellularity and vascular properties. This enables a spatially resolved prediction of how a particular patient's tumor will respond to therapy. A modified therapy can be determined based on predicted response.

### CROSS-REFERENCE TO RELATED APPLICATIONS

This application claims priority to and the benefit of U.S. Provisional Patent Application No. 63/257,740 filed Oct. 20, 2021, and to U.S. Provisional Patent Application No. 63/247,233 filed Sep. 22, 2021, the entirety of each of which is incorporated herein by reference.

### STATEMENT REGARDING FEDERALLY SPONSORED RESEARCH

This invention was made with government support under Grant numbers U01 CA142565 and U01 CA174706 awarded by the National Institutes of Health. The government has certain rights in the invention.

### FIELD OF THE DISCLOSURE

This disclosure relates generally to forecasting a response of a tumor to a therapy via quantitative magnetic resonance imaging (MRI) and biophysical, reaction-diffusion modeling.

### BACKGROUND

It is well-known that neoadjuvant therapy (NAT) in the standard-of-care setting is not optimized for each cancer patient. Currently, therapeutic regimens are based on receptor status, tumor grade, body surface area, and genetic markers (each with known shortcomings), rather than spatially-resolved physiological characteristics describing tumor features specific to the individual. While treatment plans may be altered due to lack of response, significant side-effects, or when considering the quality of life for the patient, this is implemented in an ad hoc manner. As there is no mathematical theory in place to guide such decisions, we are left with trial and error. Moreover, with certain existing clinical trial systems, it is impossible to experimentally evaluate all the possible combinations, timings, orderings, and dosing strategies for unique subsets within a cancer population, let alone for an individual patient. Therefore, effective models designed to predict tumor response in individual patients that can also be used to determine individually-optimized therapeutic regimens based on each patient's unique tumor characteristics are needed.

“Mechanism-based modeling” of cancer implies the incorporation of biological mechanisms into a model designed to predict the spatial and/or temporal dynamics of tumor characteristics. There is growing evidence that imaging-informed, biophysical mathematical models can accurately predict the development of cancers of the kidney, prostate, brain, lung, pancreas, and breast. These studies often aim to evaluate tumor growth or response to therapy on a patient-specific basis without having to first “train” the model on large population data; that is, the individual patient's data calibrates the model, followed by a model-generated prediction about that individual patient's tumor response. Imaging data is a fundamental enabler of this process as the measurements can be collected in three dimensions (3D) at the time of diagnosis and at multiple time points throughout treatment, allowing for patient-specific calibration and prediction. Such an approach not only has the capacity to potentially forecast response for individual patients, but the strategy may also enable an in silico twin to be established for each patient to test therapeutic regimens and optimize treatment. As the majority (85%) of oncology patients receive their care outside of academic research-oriented medical facilities, parameterizing models with data accessible in a community setting that is specific to an individual has the potential to dramatically and positively impact patient care.

### SUMMARY

In various embodiments, disclosed herein are protocols for a complete data acquisition, analysis, and computational forecasting pipeline for employing quantitative, magnetic resonance imaging (MRI) data to predict the response of cancers to a therapy. The disclosed approach is applicable to a heterogeneous patient population. The protocols detail embodiments for how to acquire suitable images, followed by registration, segmentation, quantitative perfusion and diffusion analysis, model calibration, and prediction. In certain embodiments, the data collection portion of the protocol may require approximately 25 minutes of scanning, post-processing may require approximately 2 to 3 hours, and the model calibration and prediction components may require approximately 10 hours per patient, depending on tumor size. In various embodiments, the response of individual cancer patients to a neoadjuvant therapy is forecast by application of a biophysical, reaction-diffusion mathematical model to this data. In various embodiments, successful application of the protocol results in co-registered MRI data from at least two scan visits that quantifies an individual tumor's size, cellularity, and vascular properties. This enables a spatially resolved prediction of how a particular patient's tumor will respond to therapy. In various embodiments, the disclosed protocols may employ an expertise in image acquisition and analysis, as well as numerical solutions of, for example, partial differential equations.

Various embodiments of the protocol involve acquiring quantitative MRI data of a cancer, potentially in community-based radiology centers, analyzing this data to return spatially resolved maps of tumor physiology, and employing the derived parameters for calibrating a predictive, mechanism-based, model of tumor growth and treatment response on a patient-specific basis. The goal of using medical imaging data to effectively inform patient-specific mathematical models of tumor growth and treatment response has been hampered by a lack of consensus on how the relevant data are collected, processed, and modeled. Embodiments of the disclosed protocol also provide a (practical) learning tool that enables future strategies to be developed that use MRI data in mathematical oncology.

The ability to accurately predict response and then rigorously optimize a therapeutic regimen on a patient-specific basis, would transform oncology. Towards this end, various embodiments provide an experimental-mathematical framework that integrates quantitative MRI data into a biophysical model to predict patient-specific treatment response of locally advanced cancer to neoadjuvant therapy. In various embodiments, diffusion-weighted and dynamic contrast-enhanced MRI data is collected prior to therapy, after one cycle of therapy, and at the completion of the first therapeutic regimen. In a study, the model is initialized and calibrated with the first two patient-specific MRI data sets to predict response at the third, which is then compared to patient outcomes (N=18). The model's predictions for total cellularity, total volume, and the longest axis at the completion of the regimen are significant within expected measurement precision (p<0.05) and strongly correlated with measured response (p<0.01). Further, the model is used to investigate, in silico, a range of (practical) alternative treatment plans to achieve the greatest possible tumor control for each individual in a subgroup of patients (N=13). The model identifies alternative dosing strategies predicted to achieve greater tumor control compared to the standard of care for 12 of 13 patients (p<0.01). In summary, a predictive, mechanism-based model has demonstrated the ability to identify alternative treatment regimens that are forecasted to outperform the therapeutic regimens the patients clinically. This has important implications for clinical trial design with the opportunity to alter oncology care in the future.

Various embodiments relate to a method comprising: acquiring, by a computing system, magnetic resonance imaging (MRI) data corresponding to a plurality of MRI scans of an anatomical region comprising a tumor of a patient, the plurality of MRI scans comprising a first set of images obtained through a first scan performed prior to an administration of a therapy to the patient and a second set of images obtained through a second scan performed following the administration of the therapy to the patient, the therapy including administration of a plurality of drugs; determining, by the computing system, from MRI data, tissue properties of tissue surrounding the tumor; registering, by the computing system, image-related data generated from the second set of images with image-related data generated from the first set of images; determining, by the computing system, diffusion characteristics of the tumor based on the tissue properties; determining, by the computing system, growth characteristics of the tumor based on the tissue properties; determining, by the computing system, for each drug of the plurality of drugs, an effect of the drug on tumor cells; and generating, by the computing system, based on the diffusion characteristics of the tumor, the growth characteristics of the tumor, and the determined effect of each drug on cells included in each voxel, a score indicating a predicted response of the tumor to the therapy.

In various embodiments, the method comprises performing tumor segmentation to identify a tumor region of interest (ROI) based on the MRI data prior to determining the tissue properties. In various embodiments, the tissue properties are related to vasculature in the tissue. In various embodiments, the MRI data comprises dynamic contrast enhanced MRI (DCE-MRI) data. In various embodiments, the tissue properties are quantified based on DCE-MRI data. In various embodiments, the tissue properties are quantified based on a pharmacokinetic model and/or based on a fluid mechanics model. In various embodiments, the tissue properties are quantified based on a Kety-Tofts model, and/or a variation of the Kety-Tofts model. In various embodiments, the MRI data comprises diffusion-weighted MRI (DW-MRI). In various embodiments, the method comprises generating a map of apparent diffusion coefficient (ADC) of water. In various embodiments, registering the image-related data from the second set of images with the image-related data generated from the first set of images aligns images with the map into a common domain. In various embodiments, the method comprises determining drug distribution in each voxel of tissue. In various embodiments, determining drug distribution in each voxel of tissue comprises generating a normalized map of blood volume to define initial drug distribution throughout the domain at time of each dose of therapy. In various embodiments, the diffusion characteristics correspond to diffusion of the tumor cells as mechanically linked to material properties of the tissue. The diffusion of the tumor cells may be mechanically linked to material properties of the tissue via a physical stressor, such as a von Mises stress. The diffusion characteristics may represent tumor changes that can cause deformations in the tissue. In various embodiments, the growth characteristics are based on a carrying capacity related to a maximum number of tumor cells that can physically fit within a voxel. In various embodiments, the growth characteristics are based on a proliferation rate per voxel. In various embodiments, the proliferation rate is calibrated per voxel within the tumor ROI for the patient. In various embodiments, the effect of each drug on tumor cells is based on concentration of the drug in the tissue. In various embodiments, the effect of each drug on tumor cells corresponds to a spatiotemporal distribution of each drug in the tissue. In various embodiments, the effect of each drug on tumor cells is based on one or more of an efficacy parameter α of the drug, a washout parameter β of the drug over time after each dose, and/or an initial concentration of the drug. In various embodiments, the efficacy parameter and/or the washout parameter are calibrated for the patient and/or the drug. In various embodiments, calibration of the washout parameter for the patient is restricted using bounds defined from ranges for terminal elimination half-lives of the drug. In various embodiments, the MRI data comprises dynamic contrast enhanced MRI (DCE-MRI) data. In various embodiments, the initial concentration is approximated using the DCE-MRI data. In various embodiments, the method comprises performing rigid and/or nonrigid intrascan registration of MRI data in each of the first set of images and the second set of images. In various embodiments, the method comprises determining a modified therapy based on the score indicating the predicted response of the tumor to the therapy. In various embodiments, the method comprises administering a modified therapy based on the score indicating the predicted response of the tumor to the therapy. In various embodiments, the therapy is a neoadjuvant therapy (NAT). Alternatively or additionally, in various embodiments, the modified therapy is a NAT. In various embodiments, the therapy is a first NAT (such chemotherapy, radiation therapy, or hormone therapy), and the modified therapy is a second NAT (a type of therapy that is different from the therapy type of the first NAT, or a different therapy of the same type as the first NAT). In various embodiments, both the therapy and the modified therapy may be prior to a surgical intervention, or both the therapy and the modified therapy may follow the surgical intervention. In various embodiments, the therapy may be prior to a surgical intervention, and the modified therapy may follow the surgical intervention.

Various embodiments relate to a computing system comprising one or more processors and a computer-readable memory with instructions configured to cause the one or more processors to: acquire MRI data corresponding to a plurality of MRI scans of an anatomical region comprising a tumor, the plurality of MRI scans comprising a first set of images obtained through a first scan performed prior to an administration of a therapy to a patient and a second set of images obtained through a second scan performed following the administration of the therapy to the patient, the therapy including administration of a plurality of drugs; determine from MRI data, one or more tissue properties of tissue surrounding the tumor; register image-related data generated from the second set of images with image-related data generated from the first set of images; determining diffusion characteristics of the tumor based on the tissue properties; determining growth characteristics of the tumor based on the tissue properties; determining, for each drug of the plurality of drugs, an effect of the drug on tumor cells; and generate, based on the diffusion characteristics of the tumor, the growth characteristics of the tumor, and the determined effect of each drug on cells included in each voxel, a score indicating a predicted response of the tumor to the therapy.

In various embodiments, wherein the instructions are configured to cause the one or more processors to perform tumor segmentation to identify a tumor ROI based on the MRI data prior to determining the tissue properties. In various embodiments, the tissue properties are related to vasculature in the tissue. In various embodiments, the MRI data comprises dynamic contrast enhanced MRI (DCE-MRI) data. In various embodiments, the tissue properties are quantified based on DCE-MRI data. In various embodiments, the tissue properties are quantified based on a pharmacokinetic model and/or based on a fluid mechanics model. In various embodiments, the tissue properties are quantified based on a Kety-Tofts model, and/or a variation of the Kety-Tofts model. In various embodiments, the MRI data comprises diffusion-weighted MRI (DW-MRI). In various embodiments, the instructions are configured to cause one or more processors to generate a map of apparent diffusion coefficient (ADC) of water. In various embodiments, registering the image-related data from the second set of images with the image-related data generated from the first set of images aligns images with the map into a common domain. In various embodiments, the instructions are configured to cause one or more processors to estimate drug distribution in each voxel of tissue. In various embodiments, determining drug distribution in each voxel of tissue comprises generating a normalized map of blood volume to define initial drug distribution throughout the domain at time of each dose of therapy. In various embodiments, the diffusion characteristics correspond to diffusion of the tumor cells as mechanically linked to material properties of the tissue via a physical stressor (e.g., a force per area), thereby representing tumor changes that can cause deformations in the tissue. In various embodiments, the growth characteristics are based on a carrying capacity related to a maximum number of tumor cells that can physically fit within a voxel. In various embodiments, the growth characteristics are based on a proliferation rate per voxel. In various embodiments, the proliferation rate is calibrated per voxel within the tumor ROI for the patient. In various embodiments, the effect of each drug on tumor cells is based on concentration of the drug in the tissue. In various embodiments, the effect of each drug on tumor cells corresponds to a spatiotemporal distribution of each drug in the tissue. In various embodiments, the effect of each drug on tumor cells is based on one of, more than one of, or all of: an efficacy parameter α of the drug; a washout parameter β of the drug over time after each dose; and/or an initial concentration of the drug. In various embodiments, the efficacy parameter and/or the washout parameter is calibrated for the patient or the drug. In various embodiments, calibration of the washout parameter for the patient is restricted using bounds defined from ranges for terminal elimination half-lives of the drug. In various embodiments, the MRI data comprises DCE-MRI data. In various embodiments, the initial concentration is approximated using the DCE-MRI data. In various embodiments, the instructions are configured to cause the one or more processors to perform rigid and/or nonrigid intrascan registration of MRI data in the first set of images and/or the second set of images. In various embodiments, the instructions are configured to determine a modified therapy based on the score indicating the predicted response of the tumor to the therapy. In various embodiments, wherein one or both of the therapy and/or the modified therapy is/are a neoadjuvant therapy (NAT).

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE DRAWINGS

FIG. 1A: Example system for implementing disclosed tumor forecasting approaches, according to various potential embodiments. FIG. 1B: Example process for tumor forecasting according to various potential embodiments. FIG. 1C: Overview of an example protocol, according to various potential embodiments. Each panel contains summary keywords for each section. The procedure has five major sections: defining the patient population (step 1, not shown), image acquisition (steps 2-9), data analysis (steps 10-25), mapping imaging data to the mathematical model (steps 26-36), and tumor forecasting (steps 37-40). Each section of the procedure focuses on specific areas of the protocol, and each section can be adapted for alternative investigations or used independently given specific circumstances in other studies. For example, the image acquisition section can be adapted for MRI studies in other organs. Also, given imaging data that is already acquired and analyzed, the mapping and forecasting sections can be applied. DW-MRI: diffusion-weighted magnetic resonance imaging, DCE-MRI: dynamic contrast-enhanced MRI.

FIG. 2: Timeline of MRI acquisition with an example standard-of-care NAT regimen for triple negative breast cancer consisting of two therapeutic regimens, according to various potential embodiments. Panel (a) depicts an example NAT regimen only, while panel (b) depicts the example regimen with the protocol's calibration and prediction strategy. For both panels, wide arrows indicate the first dose and start of each cycle (i.e., the administration of a single drug or combination of drugs over a designated period of time, typically 2-4 weeks), while the narrow arrows indicate any additional doses within each cycle. For the first regimen, dotted-line arrows represent combination doxorubicin and cyclophosphamide (typically consisting of four cycles where drugs are administered as single doses separated by two weeks). For the second regimen, solid-line arrows represent paclitaxel (typically consisting of four cycles where therapy is administered every week with each cycle lasting 3 weeks—thinner solid-line arrows represent the additional doses). Some patients are treated with carboplatin in combination with paclitaxel, where carboplatin is administered during the first week of each paclitaxel cycle only (solid-line arrows). After NAT is complete, patients undergo surgery as part of their standard-of-care to determine pathological response. The protocol has MRI data collected prior to and just after the first cycle of each therapeutic regimen.

FIG. 3: Fuzzy c-means (FCM) clustering to generate a tumor region of interest (ROI), according to various potential embodiments. Depicted is the sagittal cross section of a breast for the average DCE-MRI data for one patient (all panels). For the middle panel, a manually drawn ROI is shown, which identifies a conservative bounding polygon for the tumor. The right panel depicts the resulting ROI generated from the FCM algorithm within the manually drawn bounding polygon.

FIG. 4: Comparison of inter-visit registration results with and without tumor ROI penalties incorporated into the registration scheme, according to various potential embodiments. Panel (a) is the target image (scan 2, defined in FIG. 2), and panel (b) is the “moving” image to be deformed/shifted to align to the target image (scan 3). In panel (b), the moving image's tumor ROI is indicated by a black outline. Panel (c) presents the result of registering the moving image to the target image using the approach described in steps 27-31 of the text (i.e., rigid+non-rigid B-spline with a tumor ROI penalty). To illustrate the resulting deformations due to the registration process, Panel (d) depicts a representative grid of the original moving image, while Panel (e) presents the resulting deformed grid after registration—corresponding to the registered image in Panel (c). Panels (f) and (g) depict the deformations of the representative grid after rigid registration only (translation and rotations only) and after the non-rigid B-spline registration without a tumor ROI penalty, respectively. Across panels (b-g) white circles have been added to aid in comparing the fields for the areas surrounding the tumor. Note that by including the tumor ROI penalty, there is less deformation of the tumor ROI; i.e., there is less deformation within the white circle in Panel (e) versus Panel (g). This procedure is applied to the Scan 1, 3, and 4 MRI datasets.

FIG. 5: Flow chart of data analysis steps of an example protocol, according to various potential embodiments. Both step numbers and step names are provided. While the early data analysis steps (10-19) rely on the completion of the previous steps, several of the latter steps (20-24) can be performed in parallel.

FIG. 6: Example image acquisition results according to various potential embodiments. A central slice for an illustrative patient depicting the 200 s/mm² b-value from DW-MRI (a), the flip angle (FA) ratio from the B₁ map (b), the 10° T₁-weighted acquisitions (c), and the average signal intensity for the DCE-MRI data across all dynamics (d). The tumor burden is indicated with the box.

FIG. 7: Example results from the data analysis according to various potential embodiments. Each of the MRI data are aligned, interpolated to the same resolution, and registered across visits using a rigid registration algorithm. The DW-MRI data is used to calculate the ADC map (panels a and e). The DCE-MRI data is used to identify the tumor ROIs (panels b and f). The multi-flip angle (MFA) T₁ scans and the B₁ map correction are used to calculate a T₁ map (panels c, d, and h, respectively). The DCE-MRI data (panel b) along with the T₁ map (panel h) are used to calculate the Kety-Tofts model parameters (panel g) within the tumor ROI (from panel f).

FIG. 8: Converting imaging data to physical quantities for the mathematical model according to various potential embodiments. Prior to deriving modeling quantities, inter-visit registration is required to align the images across all visits (panel a; details provided in FIG. 4). Once aligned, the ADC maps (panel b) are used to calculate the tumor cellularity (panel c). DCE-MRI data (panel d) is used to identify fibroglandular and adipose tissues (panel e) using a fuzzy k-means algorithm. The Kety-Tofts model parameters, specific to each patient, are used along with each patient's individual therapeutic regimen (panel f) to derive approximate drug distributions in the tumor tissue (panel g).

FIG. 9: Results of the 3D model predictions (over three central slices, left column) compared to the observed results at the third scan time (right column) for one example patient according to various potential embodiments. The number of tumor cells is indicated by the color overlay on each anatomical image. Notice that the model captures the correct shape of the tumor (Dice coefficient=0.79), and while areas of higher and lower cellularity may not directly match, there are similar scales of the cellular densities between the model's predictions and the patient's actual tumor resulting in Pearson and concordance correlation coefficients of 0.80 and 0.78, respectively. Comparing the summary measures of the predicted and measured tumor at scan 3 yields 13, 17, and 4% difference in total cellularity, volume, and longest axis, respectively.

FIG. 10: Graphical depiction of the integration of breast MRI data with the mathematical model for predicting tumor response and simulating alternative treatment regimens according to various potential embodiments. The data from MRI scans obtained before and after one cycle of the first NAT regimen, are used to generate spatially resolved maps of tumor cell number and drug delivery (panels a and b, respectively). The model parameters are then calibrated using this early NAT data (panel c), and the model is run forward to the time of the patient's third scan (panel d). The model's predictions of total cellularity, total volume, and longest axis measures are then directly compared to each patient's actual tumor outcome as determined by their scan 3 data. The model's predictions were also compared to the RECIST designations to determine accuracy in the context of clinical measures. Using the patient-specific model parameters (panel c), alternative therapy regimens adjusting the frequency and dosage of treatments (“Txᵢ”, panel e) are evaluated using the model's predictions from scan 2 to 3 for each of these regimens to determine an optimal treatment schedule for each patient (panel f).

FIG. 11: Example results for the predicted response to NAT regimens for one patient (patient 4) whose standard-of-care regimen consisted of combination doxorubicin and cyclophosphamide every three weeks for 12 weeks (4 total cycles) according to various potential embodiments. The figure depicts anatomical images of a central slice of the breast overlaid with the total tumor cellularity in color. While there is not an exact match between the patient's actual scan 3 data (panel a) and the prediction for the standard regimen (i.e., the standard-of-care regimen the patient actually received, panel b), the percent differences between the predicted and measured tumor response for the standard-of-care regimen are 1%, 16%, and 1% for total cellularity, volume, and longest axis. The total cellularity predicted for two alternative regimens are also depicted: ⅓ of a dose every week (panel c), and 1/21 of a dose administered daily (panel d). (Note: each alternative regimen had the same total drug over the treatment period as the standard-of-care regimen). Across all the regimens, the model predicted the greatest tumor cell reduction when the daily dose regimen was implemented. Compared to the standard-of-care regimen, the model predicted that the daily dose regimen would result in an additional 45% reduction in total tumor cellularity.

FIG. 12: Distributions of sample averages between predicted and measured outcomes (i.e., total cellularity, volume, and longest axis) generated with a Monte Carlo resampling (N=500 for each distribution) across the patient cohort according to various potential embodiments. (See Supplemental Material below for details on the construction of these plots.) Each panel depicts the distribution of the randomly sampled differences between each predicted and measured outcome. The red vertical lines indicate the mean difference for the cohort. (Note that the red lines do not represent mean of the sampled distributions and, therefore, are not in the center of the distributions.) For example, panel a presents the sample mean differences between the predicted and measured total cellularity assuming a 10% absolute difference between predicted and measured total cellularity. Panels b and c display similar data for volume and longest dimension, respectively. Panels d-f correspond to panels a-c, but assuming a 15% absolute difference between predicted and measured total cellularity, volume, and longest axis, respectively. Panels g-i also correspond to panels a-c but assume a 20% absolute difference between predicted and measured total cellularity, volume, and longest axis, respectively

FIG. 13: Scatter plots comparing the model predictions to the actual measurements of scan 3 for each patient (N=18) along with the corresponding correlation coefficients (CCCs, PCCs, and KCCs) per group, according to various potential embodiments. The dashed line indicates the 45° line of unity. Note that for all three tumor response measures, there is greater correlation for the chemo subgroup compared to the chemo+ (i.e., patients that received chemotherapy plus targeted therapy or immunotherapy). Panel (a) depicts the comparison between the predicted total cell number to the measured total cell number as estimated from the DW-MRI data. Panel (b) depicts the comparison between the predicted total tumor volume to the measured tumor volume as determined by the total number of voxels in the tumor ROI. Panel (c) depicts the comparison of the predicted longest axis of the tumors to the actual measured longest axes as determined from the tumor ROI of scan 3. Note that using the log scale for panels (a) and (b) one patient is not shown where zero tumor was measured at the time of scan 3 (chemo subgroup, patient 4), but corresponding CC values include this data.

FIG. 14: Comparison of the percent change in total tumor cellularity from scan 2 to the time of scan 3 achieved by the standard regimen, a one-half dose double frequency regimen, a one-third dose triple frequency regimen, a one-quarter dose quadruple frequency regimen, and an equivalent daily dose when administered daily to each patient (panel a), according to various potential embodiments. Across patients, differences in the percent change between the standard therapeutic regimen and the model-identified, most effective regimen have a maximum difference of 46%, a minimum difference of 0%, and a median difference of 17% (panel b). Note: a positive difference indicates a potential additional percent reduction in the total tumor cellularity achieved by the alternative regimen compared to the standard dose the patient received. The group of standard regimens were found to be statistically inferior to the group of optimal regimens selected for each patient for tumor control/reduction (p<0.001 for percent change from scan 2 to 3 and for the predicted cell number at the time of scan 3). The most effective regimens by number of patients: standard N=1, one-half N=4, one-third N=2, one-quarter N=2, daily N=4.

FIG. 15: The MRI data goes through four different steps prior to incorporation into the mathematical model in various potential embodiments: intra-scan registration, data processing, inter-scan registration, and calculation of the modeling quantities. While more details can be found in the below text and in the figure, briefly, inter-scan registration includes alignment of the different MRI data types (panels a and b) within each scan session to correct for any possible motion that occurred during the scan visit. The data processing step includes deriving values from each of the MRI data sets to identify the region of interest (panel c), quantify the vasculature (panel d), and define values to determine cellular density of the tumors (panel e). The inter-scan registration includes aligning all of the MRI scans for each patient across time (panel f). The final step includes taking the registered data and defining the specific quantities to be used in the mathematical model including the breast region for modeling (panel g), tissue maps (panel h), tumor cell numbers (panel i), and drug concentrations (panel j).

FIG. 16: A simplified block diagram of a representative server system and client computer system usable to implement certain embodiments of the present disclosure.

FIG. 17: The two Frameworks to predict patient-specific response to NAST, according to various potential embodiments. Panel A shows the timeline of treatment administration and data acquisition for each patient. Panel B illustrates the processing-modeling pipeline to generate patient-specific digital twins. Two frameworks are established to evaluate the predictive ability of the digital twins. Framework 1 (Panel C) employs digital twins to predict the outcome of the doxorubicin and cyclophosphamide (A/C) regimen. Patient-specific images from visits 1 (V1) and 2 (V2) along with the schedule of A/C provide the input to which the digital twin is calibrated. Once calibrated, the digital twin outputs a prediction of the spatiotemporal development of the tumor in response to A/C. The prediction is then directly compared to the V3 images. Framework 2 (Panel D) employs digital twins to predict the outcome of the entire NAST. Images from V1, V2, and V3, along with the schedule of both A/C and paclitaxel, are given as input, and the digital twin outputs a prediction of whether the patient will achieve a pCR. The prediction is then directly compared to the post-surgical pathological response.

FIG. 18: Flow charts of MRI data processing, according to various potential embodiments. Panel A shows an example set of DW-MRI and DCE-MRI data acquired at one visit of a patient. Panels B-D illustrates the three steps of the processing pipeline, respectively. In panel B, multiparametric images are trimmed to the same field-of-view (FOV) and registered. In panel C, images from V1 and V3 are registered to those from V2. In panel D, tissue segmentation and calculation of tumor cellularity (from the DW-MRI data) are performed. These steps prepare the data for calibration with the biologically-based mathematical model and establishing each patient's digital twin.

FIG. 19: Temporal accuracy of patient-specific predictions of the response of TNBC to A/C, according to various potential embodiments. Panels A and B show the time courses of calibrated therapeutic efficacies over the A/C regimen in two representative patients, respectively. Panels C and D present the temporal dynamics predicted by the digital twins of the same two patients, in which subpanels (i) and (ii) represent the change of tumor cellularity (TTC) and tumor volume (TTV), respectively. In each panel, red circles present the measured TTC or TTV at certain time points, while blue curves and shadows present the predicted median and range of dynamics, respectively. Very small differences are observed between the measured and predicted changes of TTC and TTV over time in the example patients. Panels E and F plot the correlation between the measured and predicted changes of TTC and TTV (CCC=0.95 and 0.94), respectively, in the cohort. These results indicate high precision and accuracy for predicting patient-specific temporal dynamics of TNBC in response to A/C.

FIG. 20: Spatial accuracy of patient-specific predictions of the response of TNBC to A/C, according to various potential embodiments. Panels A and B show the measured and predicted tumor cell distributions on the central tumor slice for two patients. Panels C and D show the 3D renderings of the measured and predicted change of those two tumor shapes. Very small differences are observed between the measured and predicted tumor cell distributions or tumor shapes in the patients. Panel E presents the difference between the measured and predicted change of tumor cell distributions in the cohort. The median (red circle) and interquartile range (blue bar) of difference within each patient's tumor region are presented. The difference across all patients has a mean (95% CI) of 0.20% (−20.35%-20.75%). These results indicate a high accuracy of the digital twins for predicting patient-specific spatial dynamics of TNBC in response to A/C.

FIG. 21: Accuracy of patient-specific prediction of final pathological response, according to various potential embodiments. Panels A and B show the time courses of calibrated therapeutic efficacies during NAST in two example patients, respectively. Panels C and D present the temporal dynamics predicted by the digital twins for the same two example patients, in which subpanels (i) and (ii) represent the change of tumor cellularity (TTC) and tumor volume (TTV), respectively. Panel E presents the ROC analysis of differentiating pCR from non-pCR based on predicted (blue) and measured (red) TTC; Similarly, panel F presents the ROC analysis based on predicted and measured TTV. The larger AUCs of blue curves compared to red curves in both panels E and F indicates superior accuracy for predicting final pathological response

FIG. 22: Shows predicted TTC time courses (median and range) for the patient showing the largest range in FIG. 19E, according to various potential embodiments.

FIG. 23: The predicted TTV at the end of treatment (EoT), the predicted TTC at EoT, the measured TTV at V3, and the measured TTC at V3, respectively, versus the RCB-classes, according to various potential embodiments.

FIG. 24: Shows the measured and predicted tumor cell distributions on the central tumor slice for Patient 26 in FIG. 20E, according to various potential embodiments.

FIG. 25: Shows the measured and predicted tumor cell distributions on the central tumor slice for Patient 49 in FIG. 20E, according to various potential embodiments.

FIG. 26: Plots the whole-NAST prediction with the 2-scan-calibrated model and the 3-scan-calibrated model, according to various potential embodiments.

The foregoing and other features of the present disclosure will become apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, may be arranged, substituted, combined, and designed in a wide variety of different configurations, all of which are explicitly contemplated and make part of this disclosure.

Traditionally, high-spatial resolution imaging data that enables anatomical and morphological assessment has been acquired in the standard-of-care setting. Such data does not typically provide insights into the underlying physiological, cellular, and molecular characteristics of cancer, thereby limiting its use for mechanism-based, mathematical modeling. Developing more specific and quantitative measurements related to tumor biology, such as (for example) vascular status, perfusion, cellularity, hypoxia, metabolism, and proliferation is a major effort in MRI research.

In various embodiments, the disclosed protocol uses two quantitative MRI modalities: dynamic contrast-enhanced MRI (DCE-MRI) and diffusion-weighted MRI (DW-MRI). DCE-MRI acquires images in rapid succession before, during, and after the injection of a contrast agent. If the data are acquired at high enough temporal resolution (e.g., preferably 15 seconds or less per frame in various embodiments) they can be analyzed with an appropriate pharmacokinetic model to estimate different tissue vascular features on a voxel-specific basis within the imaging volume. DCE-MRI is repeatable and reproducible, and the output of DCE-MRI analysis has a statistical relationship with the response of tumors (e.g., breast tumors) to neoadjuvant therapy (NAT). In a parallel fashion, DW-MRI acquires data on water mobility in tissues that is linked to the number and quality of cell barriers present, thereby providing a noninvasive readout on tissue cellularity. DW-MRI is also repeatable and reproducible and can predict the response of tumors to NAT. These two methods may therefore be used for mechanism-based, mathematical modeling.

To predict individual prognosis for cancer patients (e.g., breast cancer patients), various embodiments employ models that use patient-specific MRI data to initialize and constrain model parameters and predictions. That is, the model parameters are calibrated to the unique characteristics of each patient. In some embodiments, a simpler logistic model that utilizes DW-MRI data may be employed to estimate tumor cellularity. In certain embodiments, the logistic model may be defined both temporally and spatially in 2D, where the baseline measurements of the tumor, tumor cell movement, and the mechanical properties of the tissues (e.g., breast tissue) are incorporated to constrain the model's predictions of the tumor growth and shape according to each individual patient's anatomy. While such a model's predictions outperform standard measures (such as the Response Evaluation Criteria In Solid Tumors, RECIST) as a prognostic indicator of response to therapy, it does not explicitly account for the therapies of each individual patient. Various embodiments thus extend the model to include estimates of drug delivery to each voxel via DCE-MRI, enabling a more accurate assessment of local tumor cell death due to therapy on a patient-specific basis. In various embodiments, this model may be used to identify theoretical treatment regimens that are hypothesized to outperform the standard-of-care regimen the patient actually received.

### Details of Model

In various embodiments, the model used is based on a 3D mathematical model that includes the mechanical coupling of tissue properties to tumor growth and the delivery of systemic therapy. The model is designed to be initialized with patient-specific imaging data to predict response of cancer patients to NAT. The governing equation (a reaction-diffusion type partial differential equation) for the spatiotemporal evolution of tumor cells N_{TC}(x̅,t), (see ‘Approximating tumor cellularity’ below), with respect to time, t, and per voxel, x̅, is:

$$\frac{\partial {N}_{T C} ( \overline{x} , t )}{\partial t} = \begin{matrix}Diffusion \\ \overbrace{∇ \cdot ( D ( \overline{x} , t ) ∇ {N}_{T C} ( \overline{x} , t ) )}\end{matrix} + \begin{matrix}Reaction \\ \overbrace{\begin{matrix}\begin{matrix}\underbrace{k ( \overline{x} ) ( 1 - \frac{{N}_{T C} ( \overline{x} , t )}{θ} ) {N}_{T C} ( \overline{x} , t )} - \\ Proliferation\end{matrix} & \begin{matrix}\underbrace{α {C}_{tissue}^{drug} ( \overline{x} , t ) {N}_{T C} ( \overline{x} , t )} \\ Death due to \\ treatment\end{matrix}\end{matrix}}\end{matrix} , \qquad ( 1 )$$

where the first term on the right-hand side describes the effects of tumor cell movement (i.e., the diffusion term), and the second term describes the tumor cell proliferation and death in time (i.e., the reaction term). All model parameters and functions are described in Table 1.

The function D(x̅,t) represents the random diffusion (movement) of the tumor cells. This function may simply be a constant resulting in isotropic tumor spread, but by incorporating individual patient anatomy into the diffusion term results in statistically significant improvements in the prediction of total tumor cell number when compared to clinical observations. Various embodiments thus mechanically couple the function D(x̅,t) to the tissue's (e.g., breast tissue's) material properties via von Mises stress σᵥₘ(x̅,t):

$$D ( \overline{x} , t ) = {D}_{0} exp ( - {γσ}_{vm} ( \overline{x} , t ) ) , \qquad ( 2 )$$

where D₀ is the diffusion coefficient in the absence of external forces, and γ is an empirical coupling constant. The exponential term damps D₀, where the von Mises stress is calculated for the fibroglandular and adipose tissues (e.g., within the breast) with the fibroglandular tissue assigned a greater stiffness than the adipose tissue. The mechanical coupling is subject to an equilibrium condition dependent upon changes in tumor cell number:

$$∇ \cdot G ∇ \vec{u} + ∇ \frac{G}{1 - 2 v} ( ∇ \cdot \vec{u} ) - λ ∇ {N}_{T C} ( \overline{x} , t ) = 0 , \qquad ( 3 )$$

where G is the shear modulus, G=E/(2(1−v)), with Young's modulus (E) and Poisson's ratio (v) describing the material properties, \vec{u} is the displacement due to tumor cell growth, and λ is another empirical coupling constant. Therefore, the diffusion term encompasses tumor changes such as growth or response to therapy that can cause deformations in the surrounding healthy tissues (i.e., fibroglandular and adipose tissues), thereby changing the stress field and the associated expansion of the tumor. It is noted that, alternatively or additionally, physical stressors other than the von Mises stress may be taken into account.

In various embodiments, the second term on the right-hand side of Eq. (1) is the reaction term that describes tumor proliferation and therapy response. Due to the nature of the data, logistic growth is defined per voxel. Specifically, for MRI data, measurements are defined per voxel, and for each voxel the volume is known. Therefore, a maximum number of tumor cells can be estimated by using an approximate cell size and packing density (see ‘Approximating tumor cellularity’ below). Using logistic growth, the carrying capacity, θ, is defined per voxel as one value for all voxels, and the proliferation rate k is spatially resolved, k(x̅), and derived from the data (see step 38).

In various embodiments, the reaction portion of the model also contains a term for tumor cell death due to therapy. The parameter α is a global parameter that represents the effectiveness of the therapy, and Cₜᵢₛₛᵤₑ^{drug}(x̅,t) represents the concentration of drug in the tumor tissue at position x̅ and time t, as approximated from the Kety-Tofts model and patient-specific parameters (see ‘DCE-MRI analysis’ below). Importantly, the concentration of contrast agent in the tissue and plasma time courses from the Kety-Tofts model now represent those quantities for the administered drug. This is achieved by replacing the concentration of contrast agent in the plasma curve with standard drug concentration curves for individual drugs and calculating the concentration in the tissue with each patient's derived vascular perfusion parameters (see ‘DCE-MRI analysis’ below). Therefore, an approximation of the concentration of drug in the tumor tissue that is spatially non-uniform and temporally varying based on the individual patient's NAT schedule is generated. Thus, the therapy term provides an estimation of the spatiotemporal distribution of drug in the tissue and its effect on the cells of each voxel. This assignment of drug delivery in the model is a first order approximation, an effort to characterize the heterogenous delivery of systemic therapy through tissue. It is noted that, in certain embodiments, this approach may implicitly assume that the chemotherapy will extravasate into the tumor tissue in a manner similar to that of the gadolinium-based contrast agent; however, this assumption may be relaxed. If a patient's drug concentration in the plasma deviates from population curves, this would affect the calibration of the model parameters; however, a provides a layer of flexibility in the model whereby the population averaged approximation of the drug in the plasma is modulated by this global parameter.

### Overview of Systems and Methods

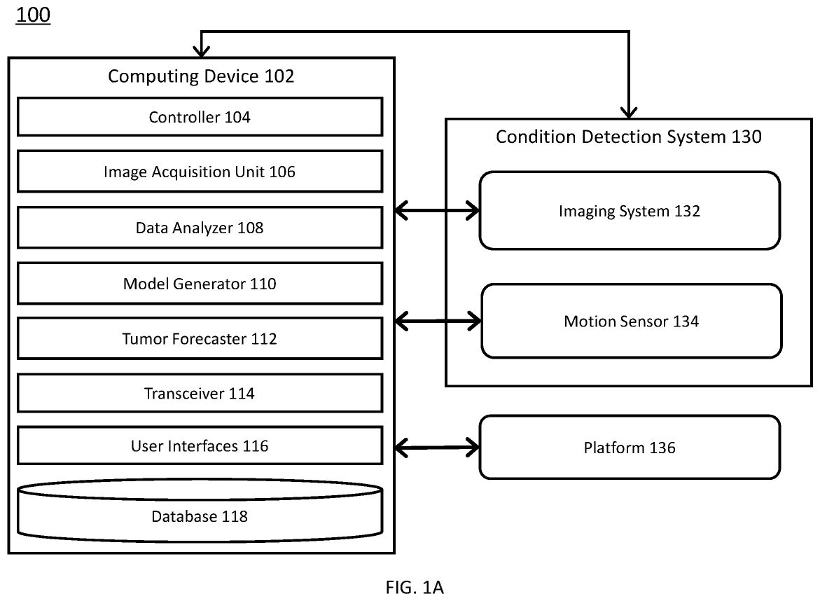

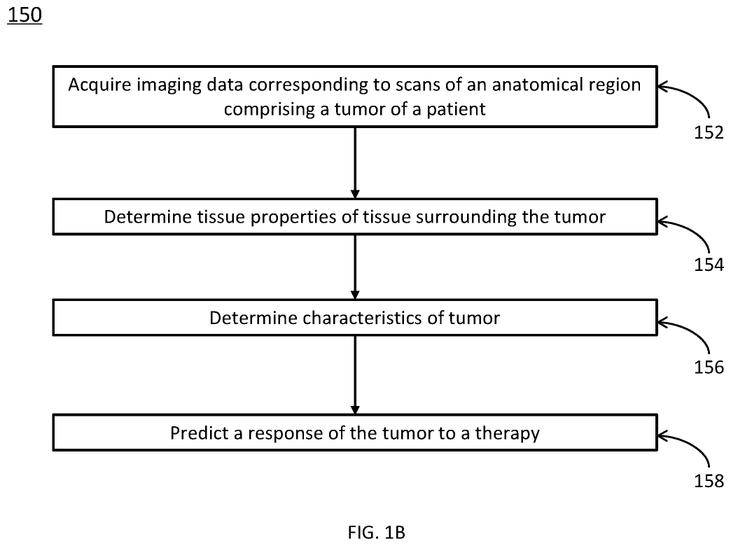

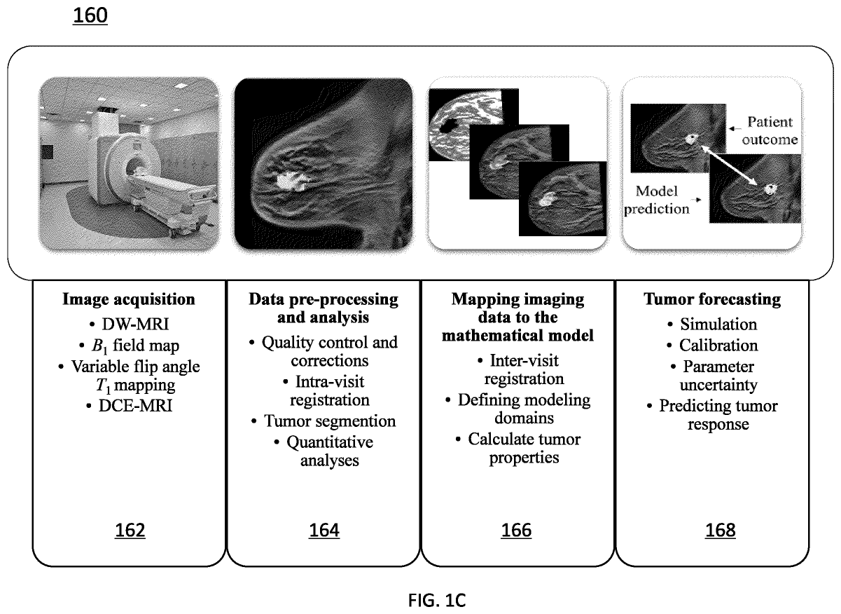

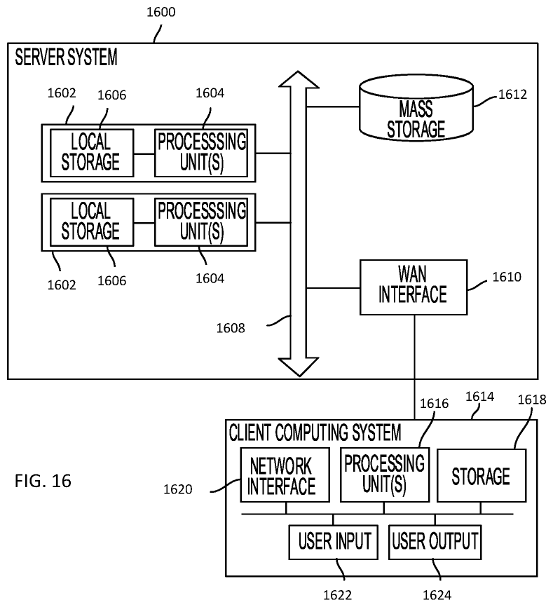

Referring to FIG. 1A, in various embodiments, a system 100 may be used to implement example protocol 160 (see FIG. 1C, and functions 162, 164, 166, and 168) and overall approach disclosed herein. The system 100 may include a computing device 102 (or multiple computing devices, co-located or remote to each other), an imaging system 132, and a motion sensor 134. The imaging system 132 may include, for example, one or more MRI scanners and/or other imaging devices and sensors capable of capturing various data so as to provide DW-MRI and/or DCE-MRI data. In various implementations, the imaging system 132 and the motion sensor 134 may be integrated into one condition detection system 130. In certain implementations, computing device 102 (or components thereof) may be integrated with one or more of the condition detection system 130, imaging system 132, and/or motion sensor 134. In various potential setups, with reference to FIG. 16, one or more of the computing devices 102 may correspond to server system 1600 that receives MRI data from a client computing system 1614 (which may be, or may comprise, an imaging system), and/or to a client computing system 1614 that sends MRI data and analyses to a server system 1600.

The condition detection system 130, imaging system 132, and/or motion sensor 134 may be directed to a platform 136 on which a patient or other subject can be situated (so as to image the subject, apply a treatment or therapy to the subject, and/or detect motion by the subject). In various embodiments, the platform 136 may be movable (e.g., using any combination of motors, magnets, etc.) to allow for positioning and repositioning of subjects (such as micro-adjustments due to subject motion).

The computing device 102 (or multiple computing devices) may be used to control and/or receive signals acquired via imaging system 132 and/or motion sensor 134 directly. In certain implementations, computing system 102 may be used to control and/or receive signals acquired via condition detection system 130. The computing device 102 may include one or more processors and one or more volatile and non-volatile memories for storing computing code and data that are captured, acquired, recorded, and/or generated. The computing device 102 may include a controller 104 that is configured to exchange control signals with condition detection system 130, imaging system 132, motion sensor 134, and/or platform 136, allowing the computing device 102 to be used to control the capture of images and/or signals via the sensors thereof, and position or reposition the subject if needed. The computing device 102 may also include an image acquisition unit 106 configured to perform, for example, image acquisition functions 162 (steps 2-9 discussed below), a data analyzer configured to perform data analysis functions 164 (steps 10-25 below), a model generator 110 configured to map imaging data to models by performing modeling functions 166 (steps 26-36), and a tumor forecaster 112 configured to perform tumor forecasting functions 168 (steps 37-40). As discussed with respect to FIG. 1C, example protocol 160 may comprise five components: defining the patient population (e.g., step 1, not shown), image acquisition (e.g., steps 2-9), data analysis (e.g., steps 10-25), mapping imaging data to the mathematical model (e.g., steps 26-36), and tumor forecasting (e.g., steps 37-40).

A transceiver 114 allows the computing device 102 to exchange readings, control commands, and/or other data with condition detection system 130, imaging system 132, motion sensor 134, and/or platform 136 wirelessly or via wires. One or more user interfaces 116 allow the computing system to receive user inputs (e.g., via a keyboard, touchscreen, microphone, camera, etc.) and provide outputs (e.g., via a display screen, audio speakers, etc.). The computing device 102 may additionally include one or more databases 118 for storing, for example, signals acquired via one or more sensors, raw and processed MRI data, and results of analyses. In some implementations, database 118 (or portions thereof) may alternatively or additionally be part of another computing device that is co-located or remote and in communication with computing device 102, condition detection system 130, imaging system 132, motion sensor 134, and/or platform 136.

With reference to FIG. 1B, an example tumor forecasting process 150 is illustrated, according to various potential embodiments. Process 150 may be implemented by or via one or more computing devices 102. At 152, the computing device 102 may acquire imaging data corresponding to scans of an anatomical region of a patient, the region having a tumor. The imaging data may be MRI data corresponding to a plurality of MRI scans of the anatomical region comprising. The plurality of scans may comprise a first set of images obtained through a first scan performed prior to an administration of a therapy to the patient, and a second set of images obtained through a second scan performed following the administration of the therapy to the patient. The therapy may comprise one or more therapies, such as chemotherapy, radiation therapy, and/or hormone therapy. The therapy may comprise administration of a plurality of drugs. In various embodiments, image-related data generated from the second set of images could be registered with respect to image-related data generated from the first set of images.

At 154, process 150 may comprise determining one or more properties of tissue that surrounds the tumor. The properties may be determined based on MRI data acquired at 152. The tissue properties may comprise mechanical or other material properties of tissue (e.g., breast tissue for breast cancer). Example tissue properties may be related to stiffness of tissue, and/or to tissue vasculature. In certain embodiments, tissue properties may correspond to shear modulus, Young's modulus, etc. In certain embodiments, the tissue properties may be known for certain tissue types, and in other embodiments, patient-specific tissue properties are determined from imaging data from scans of the patient. In certain embodiments, tumor segmentation may be performed to identify a tumor region of interest (ROI). In various embodiments, the imaging data may comprise DCE-MRI data, and the tissue properties may be quantified based on the DCE-MRI data. The tissue properties may be quantified based on a pharmacokinetic model and/or a fluid mechanics model. In certain embodiments, the tissue properties may be quantified based on a Kety-Tofts model or a variation thereof.

At 156, process 150 may comprise determining one or more characteristics of the tumor. The characteristics may comprise diffusion characteristics of the tumor and/or growth characteristics of the tumor. In various embodiments, tumor characteristics may correspond to tumor cellularity and/or tumor vasculature. In various embodiments, the characteristics may be determined based on the tissue properties.

At 158, process 150 may comprise predicting a response of the tumor to the therapy. This may comprise generating a score indicative of the predicted response. The score may be on any scale deemed suitable. In some embodiments, the score may be a likelihood (e.g., in percent) of the therapy having an intended or desired effect. Predicting the response of the tumor to the therapy may comprise determining an effect of the therapy on tumor cells. The effect of the therapy may be determined for tumor cells in each voxel. If the therapy involved multiple drugs, the effect of each drug administered as part of the therapy could be determined. The predicted response (e.g., the score) could be based on characteristics of the tumor, such as diffusion characteristics, growth characteristics, and/or the determined effect of the therapy (e.g., each drug in the therapy) on the tumor.

### Overview of Example Protocol

Various embodiments of the protocol/procedure 160 may be divided into five major components (see FIG. 1C): identification of patients who would benefit (step 1), functions 162 related to image acquisition (steps 2-9), functions 164 related to data pre-processing and analysis (steps 10-25), functions 166 related to mapping imaging data to the mathematical model (steps 26-36), and functions 168 related to tumor forecasting (steps 37-40). Each component has been divided into multiple steps for clarity of presentation. The below discussion of procedure 160 provides detailed descriptions of each component, as well as guidance on avoiding potential pitfalls and suggestions for troubleshooting.

Patient selection: In a study, an embodiment of the protocol was developed for patients recruited from community-based care centers that are eligible for NAT as a component of their care. Such patients are heterogeneous in tumor size, receptor status, age, body mass index, and ethnicity. NAT (i.e., any treatment that occurs prior to surgical intervention) is typically indicated for patients with locally advanced breast cancer and consists of one or more regimens given over the course of 4-6 months. For example, in the case of triple negative breast cancer, the standard-of-care can include doxorubicin and cyclophosphamide (first regimen), followed by paclitaxel (second regimen). There are, however, many variations in these protocols as determined by treating physicians. (This is a primary motivator for developing a mathematical forecasting system so that treatments can be optimized on a patient-specific basis.) The clinical response designations for NAT of pathological complete response (pCR) or residual disease (non-pCR) are determined by surgical pathology. Specifically, pCR is defined and reported as no residual invasive disease in either breast or axillary lymph nodes after NAT.

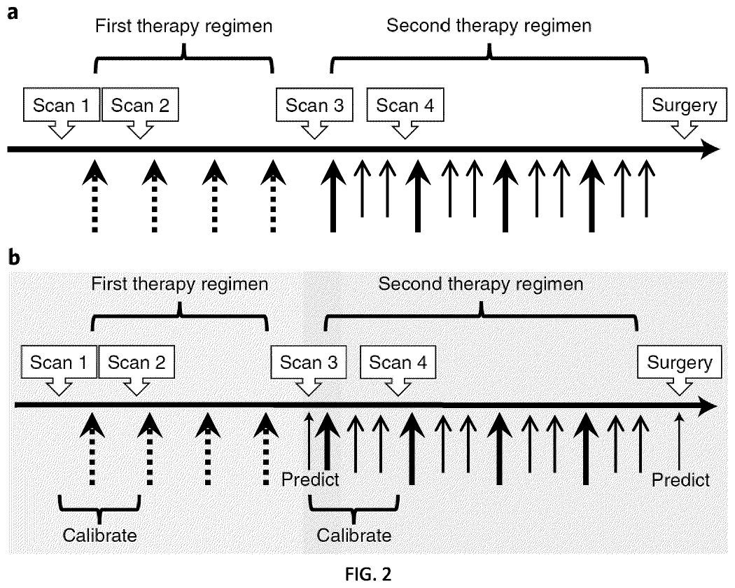

Image acquisition: In the study, the embodiment of the protocol requires the acquisition of quantitative MRI data of a breast cancer patient before and during NAT for calibrating a predictive, mechanism-based mathematical model designed to forecast their individual response. The timing of the imaging time points before and during NAT are of particular importance as they are used to calibrate, simulate, and assess predictions of tumor response with the mathematical modeling system. In certain embodiments, MRI data may be acquired at four time points: 1) prior to the initiation of NAT, 2) after one cycle of NAT, 3) after 2-4 cycles of NAT, and 4) after one cycle of NAT from scan 3. (Note: “cycle” refers to the administration of a single drug or combination of drugs over a designated period of time, e.g., 2-4 weeks.) These four time points provide data that correspond to the first cycles of each therapeutic regimen for the patients that receive two consecutive regimens (see FIG. 2). While three or more imaging time points are encouraged, two imaging time points are all that is required to calibrate the model system and then compare predictions to standard clinical measures (e.g., pathological data from biopsies or surgery) to directly test the modeling predictions.

In various embodiments, all image acquisition and patient care (imaging, oncology treatments, etc.) can be performed in community care settings (i.e., not academic, research-oriented medical centers). However, to work within the confines of imaging in standard-of-care settings certain factors should be considered. For example, in the figures and examples included in this disclosure, two scanners were used: one in an outpatient imaging facility, and the second in a regional hospital that provides both inpatient and outpatient services. While both imaging facilities undertook breast MRI as part of their routine clinical practice (a full diagnostic scan is ˜20 minutes) in the study, they are located at different sites and on different service contracts, and have different quality control guidelines. The MRI technologists at such sites are usually responsible for positioning the patients and running the research protocols, but might not have prior experience or expertise. Therefore, it is important to establish the repeatability and reproducibility of the required MRI measurements in each new environment, and implementation of the acquisition protocol requires clear (step-by-step) descriptions to be provided for the MRI technologists performing the scans.

Working with community physicians enables a broader segment of the population to be reached, but scheduling research MRI scans requires close integration and frequent communication regarding the availability of the patients, treating oncologist, referring physicians, nurses, imaging center staff, and study staff. Missing time points or lack of data due to equipment failure, patient health, scheduling issues, and/or parties unwilling to provide time is not uncommon. Moreover, community settings do not always employ a research-oriented nurse; therefore, lines of communication need to be clearly defined at the beginning of the study. Despite these challenges, working in community-based radiology settings can be easier for patients, with greater access to different facilities and allowing for more convenient travel to participate in the study.

In certain embodiments, the protocol involves acquisition of five MRI data types at each scan session: 1) DW-MRI, 2) B₁ field map to correct for radiofrequency inhomogeneity, 3) variable flip angle T₁-weighted data for generating a pre-contrast T₁ map, 4) dynamic, high-temporal resolution, T₁-weighted data before, during, and after the injection of a gadolinium-based contrast agent (DCE-MRI), and 5) high-resolution, pre-and post-contrast, T₁-weighted anatomical scans. These MRI data types were selected to provide reliable and quantitative values for individual breast cancer tumors as they have been well established in the literature. The imaging protocol utilizes standard sequences that are available on all clinical MRI scanners, eliminating the need for work in progress sequences (WIPs) or novel sequences that are not universally available.

DW-MRI provides information about the tissue microstructure by quantifying the motion of water molecules. Water molecules freely diffuse at approximately 3×10⁻³ mm²/s at 37° C., but as they encounter various tissue barriers, including large densities of cells, this diffusion rate, known as the apparent diffusion coefficient (ADC), will decrease. A minimum of two b-values (a factor that reflects the strength, duration, and timing of the diffusion-encoding gradients in the scan) is required for estimation of ADC (this protocol used three b-values of 0 s/mm², 200 s/mm², and 800 s/mm², which are commonly utilized for breast tissue). DW-MRI acquired with very high b-values (greater than 1000 s/mm²) may result in low signal-to-noise (SNR) that can adversely affect the ADC estimate, while low b-values (less than 100 s/mm²) can be affected by tissue perfusion where blood flow in the smallest vessels mimics diffusion, thereby altering the interpretation of the image.

As different tissues exhibit different T₁ relaxation times, T₁ mapping provides a means to differentiate tissue types (e.g., fat, muscle, parenchyma, and/or tumor) and provides native T₁ values needed for downstream pharmacokinetic analyses of DCE-MRI data. Various embodiments may employ a standard approach for clinical breast T₁ mapping, involving the collection of multiple T₁-weighted images at variable flip angles. Various embodiments collect images at ten flip angles ranging from 2° to 20° (in 2° increments) for estimation of typical breast tissue T₁ values (where more flip angles provide more data points for better curve fits, and this range allows for accurate estimations of various tissues in the breast from adipose to tumor). However, this approach is sensitive to inhomogeneities in the radiofrequency B₁ magnetic field used to “tip” the magnetization by various flip angles, potentially leading to inaccurate estimations of native T₁. To address this issue, a B₁ map is acquired to quantify and correct any spatial deviations in the nominal flip angle during acquisition of the T₁-weighted images used in mapping the T₁ parameter. In various embodiments, other T₁ mapping approaches include inversion and saturation recovery sequences (that are not as affected by B₁ inhomogeneities); these methods are the “gold standard” for the calculation of T₁, but the time necessary to collect these sequences in multi-slice or 3D may be clinically prohibitive and thus not incorporated into the protocol.

In DCE-MRI, a paramagnetic contrast agent is injected into the bloodstream through a peripheral vein. It travels throughout the circulatory system and can extravasate into the tumor, leading to a decreased T₁ relaxation time and corresponding increase in signal intensity in a T₁-weighted image. DCE-MRI data is acquired by collecting T₁-weighted images before, during, and after the delivery of contrast agent. DCE-MRI data can then be analyzed to segment different tissues with differing contrast enhancement and also to extract measures characterizing contrast agent pharmacokinetics (details provided below in ‘DCE-MRI analysis’). In various embodiments, acquisition parameters for the DCE-MRI measurement were selected to yield a temporal resolution<10 s (7.27 s) for accurate estimation of pharmacokinetic parameters. In various embodiments, the protocol may be adjusted for other tissue types, bearing in mind that an appropriate flip angle that minimizes contrast agent saturation effects can be selected for optimal DCE-MRI results, which may vary depending on the tissue imaged (i.e., breast vs brain).

Data pre-processing and analysis: Image processing may start with quality control, image correction, and image registration, before moving to extract tumor specific characteristics and quantitative descriptions of each tumor's cellular density and vasculature. Various embodiments involve methods including segmentation via clustering techniques as well as analysis of the quantitative MRI data to return maps of quantities reporting on blood flow and water diffusion.

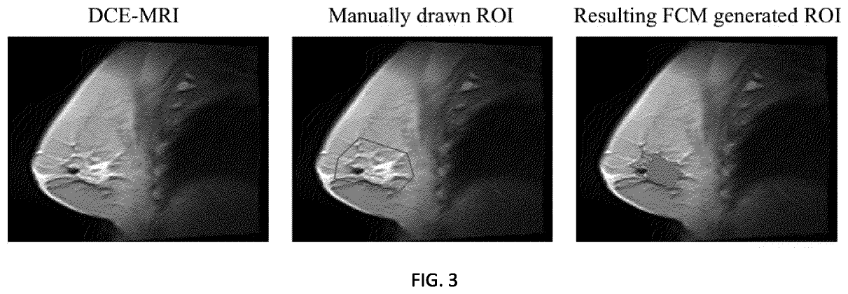

Tumor segmentation: In various embodiments, to analyze and process data, a tumor region of interest (ROI) is obtained for each patient and scan session. Some embodiments may use expertly drawn ROIs for the tumor burden. However, if provided with a conservatively drawn “bounding box” (i.e., a hand drawn polygon that surrounds the tumor, but not its specific contours), thresholding based on enhancement may be used to determine the boundaries of tumors from DCE-MRI data. This threshold is a value chosen for which any voxel with signal intensity above that threshold in the post contrast image is considered part of the tumor. As thresholding techniques can require manual adjustment for each patient scan and additional information to define patient-specific thresholds (and vary by contrast type and amount), it is preferable to use an automated approach. Various embodiments employ a fuzzy c-means (FCM) clustering algorithm. The FCM algorithm outputs the probability of a voxel being tumor or non-tumor, based on DCE-MRI contrast enhancement patterns. As FCM clustering does not partition voxels into clusters, it is more tunable compared to other “hard” clustering methods (e.g., k-means clustering). See FIG. 3 for representative images of generating ROIs using FCM.

Registration techniques: Various embodiments employ an approach to imaging-based modeling that requires that all image sets for each patient be registered to one common spatial coordinate system; i.e., the images are co-registered. Note that all registration processes do not completely preserve voxel information—even when rigid registration is used, due to multiple interpolations and re-sampling. To achieve image alignment, various embodiments may perform two types of registration: intra-visit registration (aligning all the data collected within one scan session) and inter-visit registration (aligning each of the data sets across all scanning sessions for each patient). In various embodiments, intra-visit registration is performed to correct for patient motion during the scanning session and is accomplished through a rigid registration prior to the calculation of quantitative parameters from the data.

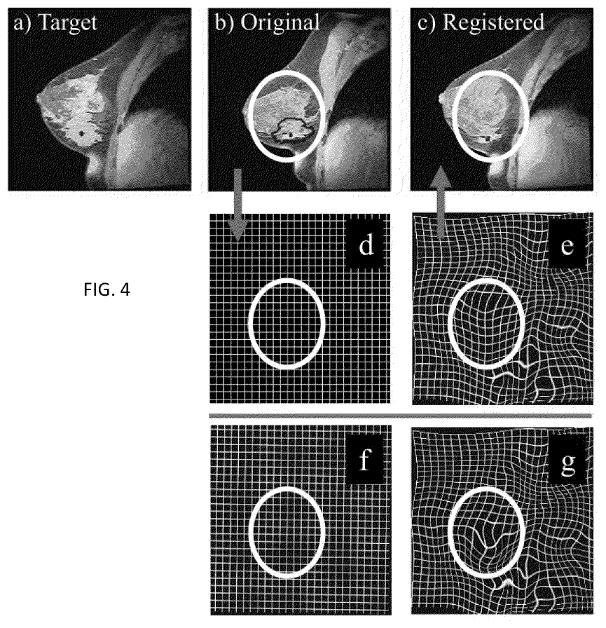

In the study, for each patient, all image data sets are registered across time (inter-visit) to a common space via a non-rigid registration algorithm with a constraint that preserves the tumor volumes at each time point. If MRI data is obtained at four time points, various embodiments may choose to register scans 1, 3, and 4 to scan 2 (target), as scan 2 is not an endpoint scan and is acquired early enough so that the patient will (likely) have tumor burden present to help guide alignment. In some embodiments, the registration algorithm may include, or may consist of, a rigid registration of the tumor ROIs followed by a deformable b-spline registration with a rigidity penalty on the tumor regions. This rigidity penalty is imposed to preserve the tumor volume/size and shape across all scan times. With a fully deformable registration, the tumor ROIs of scans 1, 3 and 4 may be morphed to match the tumor ROI of scan 2. See FIG. 4 for example comparisons of different registration results.

DCE-MRI analysis: In various embodiments, the DCE-MRI data is analyzed using models of contrast agent pharmacokinetics to derive quantitative parameters of vascular perfusion and tissue volume fractions. For example, the extended Kety-Tofts model (or a variation thereof) is used to perform quantitative analysis of DCE-MRI data. In various embodiments, temporal resolution of about 7.27 s for DCE-MRI data provides sufficient SNR and temporal sampling for the extended Kety-Tofts model to be applied. However, various embodiments include evaluation of voxel time course fits to determine which model appropriately captures the time course behavior of specific data sets. Pharmacokinetic modeling requires characterization of the time rate of change of the concentration of contrast agent in a feeding artery, i.e., the arterial input function (AIF). Various embodiments estimate the AIF from the population averaged signal intensity time course extracted from the axillary artery. Further, various embodiments calculate the bolus arrival time (BAT) to shift the population AIF on a voxel-wise basis to align the enhancement time of the AIF with that of the individual voxel, allowing for improved fits and more accurate parameter estimation.

In various embodiments, the reference region model is another approach to quantitative DCE-MRI analysis, removing the need for direct measurement of an AIF, where the tumor enhancement curve is compared to that of a reference region, such as pectoral muscle tissue (requiring additional tissue segmentation efforts). In various embodiments, simpler methods to analyze DCE-MRI data, such as the calculation of the signal enhancement ratio and area under the signal intensity time course curve, can provide semi-quantitative measures of vascular characteristics that have proven informative in distinguishing benign and malignant lesions and predicting disease recurrence.

Approximating tumor cellularity: In various embodiments, the ADC is calculated from the DW-MRI data, representing the rate at which water molecules diffuse in the tissue. It has been shown to approximate the cellular density of tissue. ADC values may be calculated for each voxel via standard methods, and there is an inverse relationship between the measured ADC and tumor cellularity. However, the source of changes in ADC may vary, as many other factors (e.g., cell membrane permeability, cell size, and tissue tortuosity) in addition to cellularity can also affect the measured ADC. Therefore, the approach of appraising cellularity with the ADC may be an approximation, and other methods for estimating ADC with less ambiguity to improve outcomes from applying the protocol.

Mapping imaging data to the mathematical model: In various embodiments, after the generation of quantitative maps from each MRI data type, the maps are registered across all scan sessions (i.e., inter-visit registration) for each patient so that all imaging data is aligned to a common image space. Once aligned, a tissue domain (e.g., breast domain) is defined, within which each individual patient's data is used to calculate physical characteristics of each patient's tumor utilized by the mathematical model. The steps for generating tumor characteristics include calculating tumor cellularity, defining masks delineating fibroglandular and adipose tissues, approximating drug distributions, and estimating summary measures for each tumor across all scans.

In various embodiments, the mathematical model employs the patient-specific characterization of breast (or other tissue) anatomy, cellularity, vascular features, and approximate drug distributions. If a mathematical model, initialized and constrained by non-invasive imaging data for an individual patient collected early in the course of therapy, could be used to reliably predict tumor response, oncologists may be able to intervene and modify therapy on a patient-specific basis. Additionally, such a model could be used to optimize therapeutic regimens for patients with more robust mathematical methods such as optimal control theory.

Tumor forecasting: In various embodiments, the quantitative maps are then used to initialize and calibrate tumor cell proliferation, drug efficacy, and cellular migration within the mathematical model. That is, each patient's own imaging series is used to parameterize or identify growth and response parameters unique to that individual patient. Once parameterized, the model can be run forward for patient-specific predictions of the spatio-temporal evolution of tumor cellularity, allowing for a prediction of treatment response that can be directly compared to the observed outcome for each patient.

### Implementing the Protocol

Various embodiments of this protocol may be implemented by a multi-disciplinary team with experience and expertise across multiple fields including both the acquisition and analysis of advanced, non-standard-of-care MRI data in the clinical setting, image processing (including segmentation and registration), as well as the numerical solution of partial differential equations (PDEs). If implemented in the community setting, implementation may involve coordination with community health providers.

In various embodiments, the procedure described below addresses challenges presented for the incorporation of cancer (e.g., breast cancer) MRI data into predictive mathematical models. These methods are thus applicable to data collected in the community setting, across multiple imaging sites, within specific standard-of-care constraints, and for a heterogeneous patient population. The repeatability and reproducibility of this quantitative MRI protocol at community-based imaging centers has been established. Furthermore, embodiments of the protocol utilize several strategies to reduce bias and dependency on user interaction, including detailed data acquisition strategies, as well as automated or semi-automated computational algorithms. Certain tasks would benefit from expert evaluation (e.g., outlining tumor regions of interest by radiologists) and others that rely on the operator's input (e.g., positioning the field-of-view for MRI acquisition). Embodiments of the disclosed approach include automated quality checks and quantification of the data to reduce overall user/operator influence.

### Applications of Protocol

In various embodiments, the protocol is well-suited for predicting response in cancer patients receiving NAT as a component of their clinical care. While certain details of the protocol presented here are specific to breast cancer, the methods are generally applicable to any solid tumor for which the requisite data is accessible. There are also applications of the disclosed approach in the pre-clinical setting.

In various embodiments, in addition to the mechanism-based, mathematical modeling for which the protocol is well-suited, investigators who have previously collected the appropriate imaging data may use portions of the protocol for data processing and analysis to yield (for example) longitudinally aligned quantitative maps of tumor features. The data returned at the completion of step 24 can then be employed in more conventional statistical studies that assess longitudinal changes in tumor characteristics and separating responders from non-responders. Such data can also provide the input data for analysis in the field of radiomics, “habitat imaging,” as well as for application in artificial intelligence-based models.

### Considerations

The temporal sampling requirements of the DCE-MRI protocol constrains the achievable spatial resolution of data that can be collected while limiting noise. All images are acquired in the sagittal plane; while the transverse plane is the standard-of-care choice with a bilateral field-of-view (FOV), embodiments of the disclosed imaging protocols use sagittal slices for better resolution over the affected tissue within a minimal amount of time. For example, a standard-of-care, T₁-weighted, contrast-enhanced acquisition requires 80-90 sec, whereas the same scan in embodiments of the disclosed approach is less than 10 seconds. With this coarser spatial resolution, important anatomical features such as small feeding vessels may potentially be missed. This, in turn, can affect modeling strategies that aim to incorporate nutrient/oxygen delivery as well as estimates of the distribution of systemic therapies. However, faster acquisitions at the spatial resolution and FOV typically acquired in the standard-of-care setting would be expected to enhance results.

Because some error is expected with quantitative measurement, various embodiments employ best practices for encountering and dealing with physiologically implausible results—such as during the interpretation of diffusion-weighted data and pharmacokinetic analysis. This may be of particular importance because mathematical models project/propagate errors derived from the data employed for calibration and prediction (which are addressed in the tumor forecasting section, steps 37-40). The MRI data and corresponding quantitative analyses are ultimately approximations of the breast cancer's characteristics at the tissue and cellular scale, and alternative methods and procedures may be employed to reduce error and ambiguity in these quantities.

It is emphasized that the protocols and approach presented herein are examples, and alternative embodiments may employ one of many other possible modeling formulations applicable to the data set produced by this pipeline, and there are various other cancer modeling styles. Also, while embodiments of the disclosed protocol are built to accommodate heterogenous populations of patients, there are certain exclusions applied to make the mathematical modeling as practical as possible. For example, a study excluded tumors at stages I and IV due to the fact that stage I tumors may be too small to reliably measure (<2 cm in diameter) with the MRI techniques, and stage IV may require alternative modeling techniques to account for tumor invasion and metastasis. Also, stage I and IV patients do not typically receive NAT—stage I tumors are treated with surgery and radiation, and stage IV tumors are treated with palliative care. Furthermore, it is noted that many programming languages and numerical schemes are available for determining solutions for PDEs (such as the finite element method), and for those less interested in the derivation of numerical codes, specific software programs exist to aid in the implementation of PDEs (e.g., FEniCS and MATLAB's PDE solvers).

### Study Materials

Subjects: Women over the age of 18 who present with intermediate to high grade invasive primary breast cancer and are considering NAT as a component of their clinical care. Patients with disease from all subtypes and treatment regimens (including immunotherapy, targeted, and cytotoxic therapies) may be included if they have disease stage II and III cancers. Patients with a history of kidney disease, abnormal creatinine or estimated glomerular filtration rate, who are pregnant or who are breast feeding must be excluded. Also exclude any patients who have any non-MR compatible ferromagnetic materials, are acutely ill, and/or for whom an MRI is technically unfeasible (e.g., due to breast volume or obesity). For the example data presented here, health information related to each participant's disease and MRI scans were obtained. This information includes laboratory test results, medical imaging reports, and diagnosis and treatment codes.

Reagents: Gadolinium-based contrast agent (e.g., Multihance (Bracco, Monroe Township, NJ) or Gadovist (Bayer, Leverkusen, Germany)). Used for contrast-enhanced scans.

Equipment: Power injector for administration of contrast agent (e.g., Medrad, Warrendale, PA), MRI Scanner, and personal computer or server. For the data processing and analysis as well as the model simulations, a personal computer may be capable of running the model calibration via the software described below, but a server with specifications such as those presented here, with parallelized scripts for computational efficiency, may be preferred for various embodiments: 40 nodes; CPU per node: 2/8 Xeon E5-2680 2.7 GHz (turbo, 3.5) 1/61 Xeon Phi SE10P 1.1 GHz; memory: 32 GB per node.

MRI Scanner Setup: MRI of the breast may be acquired using 3T scanners equipped with an 8- or 16-channel double-breast receive coil. Studies discussed herein employed Siemens scanners (Siemens Healthcare, Erlangen, Germany) with Sentinelle coils (Invivo, Gainesville, Florida). Other 3T scanners (Philips, GE) and breast coils may require slight adjustments to replicate the pulse sequences described in section 3.2, but as these are standard sequences, the scanners have the capabilities of collecting equivalent imaging protocols. After scanning, Digital Imaging and Communications in Medicine (DICOM) files are stripped of protected health information (PHI), labeled with assigned study identifiers, and uploaded onto a firewalled protected server.

Software Setup: REDCap (https://www.project-redcap.org/) or similar. Clinical information regarding patient demographics, diagnosis, treatment, and surgical outcome may be relayed to the research team and stored in REDCap, a secure, HIPAA-compliant web application for building and managing online databases. REDCap is a widely available and utilized service for managing single and multi-institutional clinical studies.

MATLAB (MathWorks, Natick, MA) or similar. In various embodiments, MATLAB software may be used for the vast majority of processes due to its portability and convenient functionality across a diverse group of biomedical researchers including experimentalists, computational scientists, engineers, physicists, mathematicians, and medical professionals. However, alternative software programs are available for the data processing, and with a sufficient programming background, the functions described throughout the disclosed protocol can be replicated in those environments.

Elastix (https://elastix.lumc.nl/). Elastix is a toolbox for rigid and nonrigid registration of images in 3D. Elastix is an open-source toolbox, and its functions can be run through the command line via MATLAB allowing it to be integrated into existing data processing scripts.

### Study Procedure

Patient selection, timing˜15 minutes:

Step (1). Ensure patient is eligible for the study. Patients should be women over the age of 18 who present with intermediate to high grade invasive primary breast cancer and are considering NAT as a component of their clinical care. Verify with the treating oncologist that each patient has no history of kidney disease and has normal creatinine and estimated glomerular filtration rate within 30 days of imaging studies. Exclude pregnant women and women who are breast feeding. Also exclude any patients who have any non-MR compatible ferromagnetic materials, are acutely ill, and/or for whom an MRI is technically unfeasible (e.g., due to breast volume or obesity).

Step (2). Obtain informed consent from patient. Patients should be asked to consent to participate in research. Quantitative MRI, PHI, and therapeutic regimens are acquired for patients diagnosed with intermediate to high grade invasive breast cancers who are eligible for NAT as a component of their clinical care at community-care oncology sites. The study population consists of women over the age of 18 who present with primary breast cancer and are considering NAT as a component of their clinical care. Importantly, patients with disease from all subtypes and treatment regimens (including immunotherapy, targeted, and cytotoxic therapies) may be included for disease stage II and III cancers.

Image acquisition, timing˜1.5 to 2 hrs total per session (patient arrival to departure), 40 min (patient placement and removal from scanner), ˜25 min (MRI scanning). Forty minutes may allow sufficient time for patient to be positioned in the breast coil (prone position), scanned, and exited from the scanner. Preparation for the MRI examination is may be a typical, standard-of-care exam, including the placement and removal of an intravenous line for administration of the contrast agent.

Step (3). Insert an intravenous line. A short peripheral intravenous catheter (20-22 gauge) is inserted in the antecubital or forearm area. Correct positioning of the catheter tip should be checked for venous backflow by withdrawing blood and flushing with normal saline. Please see Table 2 for a complete summary of the imaging parameters described in the following steps.

Step (4). Establish the FOV. Obtain several localizer scans at the beginning of the MRI session. First obtain a localizer scan by acquiring a three-plane (axial, sagittal, and coronal) scout image series covering both breasts. Adjust the FOV to ensure coverage of the tumor in the affected breast without changing scan parameters that would affect resolution or scan time. As all patients have been diagnosed with breast cancer and tumor location is known it is helpful to identify the approximate location of the lesion (e.g., ‘7 o'clock, right breast’) from the referring physician's office prior to the MRI to aid FOV placement. Often, a metal-induced susceptibility artifact around the biopsy clip/marker can be used to locate the approximate tumor center on the localizer scans. While finding the center of the FOV, adjust the coverage in the anterior-posterior direction to include the axillary artery whenever possible (to allow for characterization of the AIF). Acquire a second localizer scan with a sagittal-only sequence with 30 slices each 5-mm thick (15 cm of patient right to left coverage). The imaging volume is centered on the tumor and all or as much of the tumor as possible is included.

Step (5). Acquire DW-MRI data. Acquire DW-MRI over 10 slices with 5 mm thickness and no slice gap. The following parameters may be used: repetition time/echo time TR/TE=3000/52 ms, flip angle 90°, matrix 128×128 (over a 256×256 mm² field-of-view), and GeneRalized Autocalibrating Partial Parallel Acquisition (GRAPPA) acceleration of 2. The study includes spectrally selective adiabatic inversion recovery (SPAIR) fat suppression. To allow for approximately equal SNR ratios at all b-values, average six acquisitions for b-values of 0 and 200 s/mm², and average 18 acquisitions for a b-value of 800 s/mm² for a monopolar, single-shot spin echo, echo planar imaging sequence in a 3D-diagonal diffusion-encoding direction. If using other scanners use a sequence comparable to GRAPPA (e.g., ARC (Autocalibrating Reconstruction for Cartesian imaging) for GE, and image-domain SENSE (SENSitivity Encoding) for Phillips.

Step (6). Map the B₁ field. Use the Siemens TurboFLASH sequence with a pre-conditioning RF pulse to map the B₁ field with the following acquisition parameters: TR/TE=8680/2 milliseconds, flip angle=8°, matrix 96×96, and slice thickness 5 mm. Due to the inclusion of a slice gap in the B₁ mapping protocol, perform two acquisitions with a 5 mm slice gap, to cover the same FOV as the above measurements without missing coverage in the slice direction. This pulse sequence is only available as a standard product sequence on Siemens scanners at the time of this printing. When Philips or GE Healthcare scanners are used, an approximate B₁ map can be computed from assuming a uniform T₁ over adipose tissue and interpolating over the rest of the images as published previously.

Step (7). Acquire a high-resolution T₁-weighted image. Use a VIBE (Volumetric Interpolated Breath-hold Examination; no breath-holding required) sequence with the following acquisition parameters: TR/TE=5.3/2.3 milliseconds, flip angle 10°, acquisition matrix 256×256, slice thickness 1 mm (96 slices), GRAPPA acceleration of 2 in the phase encode direction, and SPAIR (Spectral Selection Attenuated Inversion Recovery) fat suppression. Then acquire images for the pre-contrast T₁ map without fat suppression with T₁-weighted images from the 3D gradient-echo, FLASH (fast low angle shot) sequence, also known as an SPGRE (spoiled gradient recalled echo) sequence, at 10 flip angles (2, 4, 6, . . . 20°) with the following parameters: TR/TE=7.9/2.71 ms and GRAPPA accelerating factor of 3 in the phase encode direction. Use an acquisition matrix of 192×192×10 over a sagittal square FOV (256 mm²) with slice thickness of 5 mm. This pre-contrast T₁-weighted MRI scan with fat suppression is required for anatomical visualization purposes as well as for radiologists to identify the tumor ROI. The pre-contrast T₁ map is required for pharmacokinetic modeling of the DCE-MRI data described in the next step.

Step (8). Acquire a dynamic set of T₁-weighted, VIBE (no breath-holding required) images (this is the DCE-MRI acquisition). Use the following acquisition parameters: TR/TE=7.02/4.6 ms a flip angle=15°, matrix 192×192, with 10 slices of 5 mm thickness each, and GRAPPA acceleration factor of 2 in the phase encode direction yielding a temporal resolution of 7.27 seconds. (Note that, with further developments in rapid acquisition, slices thinner than 5 mm may be accessible without sacrificing too much SNR.) The comparable sequences on GE and Philips scanners are FAME (Fast Acquisition with Multiphase EFGRE3D) and THRIVE (T1W High Resolution Isotropic Volume Examination), respectively. Start acquisition of this DCE-MRI data. After 1 minute begin to administer a gadolinium-based contrast agent via a power injector at the dosage recommended on the product insert and at the flow rate of 2 mL/sec through the IV catheter placed in step 3, all while continuing to acquire the DCE-MRI data. Follow administration of the contrast agent with a saline flush (20 mL) at a flow rate of 2 mL/sec through (again) the IV catheter placed in step 3. Note that different contrast agents or injection rates may lead to different pharmacokinetics. If pharmacokinetic parameters are calculated, used, or compared across the patient population, it is important to use a consistent contrast agent and injection protocol. It is also important to record the contrast agent dosage used for reference at subsequent visits.

Step (9). Acquire a post-contrast, high-resolution T₁-weighted image. Use a VIBE (no breath-holding required) sequence with the following acquisition parameters: TR/TE=5.3/2.3 milliseconds, flip angle 10°, acquisition matrix 256×256, slice thickness 1 mm (96 slices), GRAPPA acceleration 2, and SPAIR (Spectral Selection Attenuated Inversion Recovery) fat suppression. A post-contrast, T₁-weighted MRI scan with fat suppression is required for anatomical visualization purposes as well as for radiologists to identify the tumor ROI.

Image analysis, timing˜2-3 hrs:

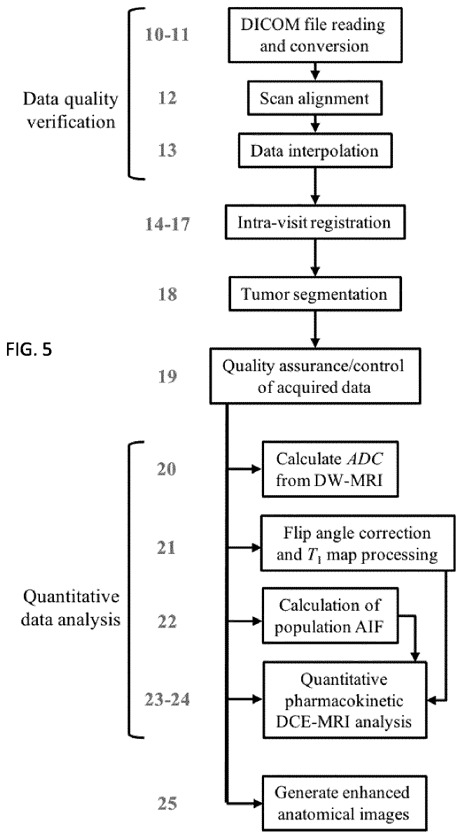

Step (10). Upload and save data. Save as un-anonymized DICOM files onto the Picture Archiving Communication System (PACS) server associated with the radiology practice that is acquiring the scans. Save a separate copy of the DICOM files for which PHI is replaced with a unique subject identifier and upload these anonymized files onto a firewalled protected server. For each patient, it is advisable that the images be processed and analyzed as a set across all visits. See FIG. 5 for a flow chart of all steps in this data analysis pipeline.

Step (11). Organize data. Copy DICOM data into individual patient visit directories to ensure original copies of the data taken off the scanner are preserved. Import DICOM data into the MATLAB workspace using built-in MATLAB functions dicomread and dicominfo. Arrange DICOM slices in ascending order of slice position within the scanner (i.e., not acquisition order but spatial order; slices collected in an interleaved fashion should be reordered by spatial location). Save the final image for each type of MRI scan as a matrix in a MATLAB structure, along with the header information for each DICOM (pulled in using dicominfo). Save each scan's structure in floating point precision as .mat files to integrate with the pipeline's image processing and analysis steps. Save the DW-MRI, variable flip angle T₁-weighted and DCE-MRI data as 4D matrices where the fourth dimension represents each b-value, flip angle, and repetition, respectively. Save the final MATLAB structure to each patient/visit directory.

Step (12). Check the slice positions. Using the DICOM header information so that each scan is aligned with those of the DCE-MRI data to ensure the same FOV is being analyzed across scans for each patient and visit. At the scanner, several types of errors can occur including a shifted FOV (compared to previous scans) or slices offset by a value different than the 5 mm slice thickness.

Step (13). Up-sample the DW-MRI and B₁ map data to match the DCE-MRI spatial resolution. Use a nearest neighbor interpolation for 2D gridded data (interp2, MATLAB). Save the resulting interpolated data to the .mat file in the MATLAB structure for that patient and visit. In this protocol the B₁ map and DW-MRI data is acquired at a lower spatial resolution than that of the DCE-MRI data (due to time constraints and SNR considerations). These scans should match the resolution of the target image for intra visit registration.

Step (14). Align DW-MRI data to DCE-MRI data. Use the b=0 image (as the SNR is higher for the b=0 s/mm² data compared to the b=200 or 800 s/mm² data). Register each slice of the b=0 s/mm² diffusion-weighted scan to the corresponding slice of the first repetition of DCE-MRI data using the MATLAB rigid registration algorithm with function imregtform. Apply the transformation obtained from the function's output to the b=200 and 800 s/mm² DW-MRI data using the MATLAB imwarp function. Save the resulting 4D matrix of DW-MRI data registered to the DCE-MRI data as a separate .mat file.

Step (15). Align the variable flip angle T₁-weighted MRI to the DCE-MRI data. Repeat step 14, but use the variable flip angle T₁-weighted MRI data in place of the DW-MRI data. Register each slice of each T₁-weighted image to the first repetition of DCE-MRI data (imregtform, imwarp, MATLAB). Save the variable flip angle data as a 4D matrix and then export as a .mat file.

Step (16). Align B₁ maps. The B₁ mapping sequence utilized for this study outputs a proton-density weighted image and a calculated map of the estimated flip angle each voxel in the image experienced. To align the B₁ maps to the DCE-MRI data, register each slice of the proton-density weighted image to the first repetition of DCE-MRI data (imregtform, imwarp, MATLAB—see step 14) due to the higher SNR of the proton-density weighted image compared to the flip angle map. Apply the resulting geometric transformation to the flip angle map. Save the resulting data and export as a .mat file.

Step (17). Correct for motion within the DCE-MRI scan. Align each slice of each repetition of the DCE-MRI sequence to the corresponding slice of the first repetition (imregtform, imwarp, MATLAB—see step 14). Save the resulting data as a 4D matrix and export as a .mat file. Breast motion due to respiration or patient movement, can occur during the DCE-MRI acquisition.

Step (18). Generate tumor ROIs. Manually draw a bulk ROI (this can be done by a board-certified radiologist) to conservatively outline the lesion under investigation. Then apply FCM clustering to the voxels within the drawn ROI with the class number set to two (one each for the lesion and non-lesion tissue types) using MATLAB's fcm function. After the binary mask is identified, post-process to fill holes (i.e., zeros in the mask that are surround by ones) via the MATLAB function imfill. Additionally, eliminate regions less than 8.45 mm³ (i.e., 1×1×1 voxels) via the MATLAB function bwareaopen. Save the resulting ROI to the .mat file in the MATLAB structure for that patient and visit.

Step (19). Ensure registration is successful and identify any artifacts (due to silicone implants, cardiac motion, etc.). Manually visualize each set of pre-(from steps 11-13) and post-registration (from steps 14-17) scans. Remove patient data sets with substantial artifacts (deforming the images) from the study. To evaluate the results of tumor segmentation, visualize ROIs by overlaying them on background subtracted DCE-MRI data as well as high-resolution post-contrast T₁-weighted images.

Step (20). Calculate ADC maps. Fitting the DW-MRI data to Eq. (4):

$$S ( b ) = {S}_{0} \cdot exp ( - ADC \cdot b ) , \qquad ( 4 )$$

where S(b) is the signal intensity in the presence of diffusion gradients at strength b, S₀ is the signal intensity in the absence of diffusion gradients, and b is the strength of the diffusion gradients. Fit Eq. (4) to the signal intensities from the b=200 and b=800 s/mm² DW-MRI data on a voxel-by-voxel basis. In particular, for data sets that have only two b-values, the ADC values can be calculated directly (i.e., no fitting). Alternatively, use linear regression with ln(S(b)) and b to determine the ADC map via MATLAB function regression. While the b=0 s/mm² data is acquired, it is not used in the ADC calculation for this protocol—due to low b-values being affected by tissue perfusion—but is used in the registration of the diffusion-weighted data (details in step 14). Save the resulting ADC map to the .mat file in the MATLAB structure for that patient and visit.

Step (21). Calculate the B₁-corrected T₁ values for each voxel. Fit (lsqcurvefit, MATLAB) the measured multi-flip angle, signal intensity data to the gradient recalled echo signal, S, equation:

$$S = {S}_{0} [ \frac{sin ( f \cdot a ) \cdot ( 1 - exp ( - TR / {T}_{1} ) )}{1 - ( exp ( - TR / {T}_{1} ) \cdot cos ( f \cdot a ) )} ] exp ( - TE / {{T}_{2}}^{*} ) , \qquad ( 5 )$$

where S₀ is a constant related to scanner gain and proton density, a is the prescribed set of flip angles, and f is the flip angle correction factor for a given voxel that accounts for inhomogeneity in B₁ (TE<<T₂* is assumed, so exp(−TE/T₂*) can be set to 1). The flip angle correction factor (f) is calculated by dividing the B₁-map by the flip angle (a). The fitting process yields the flip-angle corrected T₁ map and S₀. Save the resulting corrected T₁ map to the .mat file in the MATLAB structure for that patient and visit.

Step (22). Calculate the population AIF. For each patient's data set, generate a subtraction image from the contrast-enhanced data where the average pre-contrast dynamics are subtracted from the average post-contrast dynamics. For each scan, identify the axillary artery in the subtraction images. Manually select a single voxel within the axillary artery as a “seed” for a 3×3 kernel centered on the seed. Save the location of the seed in a vector. For an adjacent slice, select a 5×5 ROI centered on the voxel corresponding to the seed voxel location. For each voxel within the 5×5 ROI, define a 3×3 window centered on each voxel. Compare the average signal intensity time courses of each window (25 windows) to that of the seed voxel's kernel using the correlation coefficient (MATLAB's function corr). For the voxel window with the greatest correlation to the seed, save the location of the voxel in the vector and set the voxel as the new seed. Repeat this process through all of the slices. For the voxels saved in the vector, remove any voxels with the following criteria (i)-(iii):
(i) the maximum signal intensity does not occur within the first (approximately) 45 seconds of the post contrast dynamics
(ii) the maximum signal intensity is <(approximately) 20 times greater than the standard deviation of the first three pre-contrast dynamics
(iii) the average signal intensity of the last (approximately) 120 seconds are >(approximately) 40% of the maximum

For the voxels that remain in the vector after the three bulleted steps, average them at each time point to yield one single, average time course. Save the resulting time course as the individual AIF to the .mat file in the MATLAB structure for that patient and visit. To generate the population AIF, average the resulting individual AIFs across all patient sets available. This calculation of the population AIF makes use of the semi-automatic algorithm developed by Li et al. Calculation of the population AIF can be updated with each acquisition of new patient data.

Step (23). Determine the BAT. Fitting the signal intensity time course from each tumor voxel (using MATLAB's lsqcurvefit) to the half-logistic function

$${S}_{hl} ( t ) = \{ \begin{matrix}0 , & 0 < t \leq {τ}_{A T} \\ \frac{2 A}{1 + exp ( - B ( t - {τ}_{B A T} ) )} - A , & t > {τ}_{A T}\end{matrix} , \qquad ( 6 )$$

where A and B control the amplitude and slope of the uptake portion of the Sₕₗ(t) time course, respectively, and τ_{BAT} is the BAT at a specific voxel location. Temporally align the population AIF curve for each voxel with:

$${C}_{p} ( t ) = \{ \begin{matrix}0 , & 0 < t \leq {delay}_{pop} \\ AIF ( t - {delay}_{pop} ) , & t > {delay}_{pop}\end{matrix} , \qquad ( 7 )$$

where delayₚₒₚ=τ_{BAT}−τₚₒₚ and τₚₒₚ is the BAT of the population AIF.

Step (24). Convert the measured signal intensity for the DCE-MRI experiment to the concentration of contrast agent to enable the pharmacokinetic modeling described below. The signal intensity measured in the DCE-MRI experiment is described by Eq. (5), where we note that T₁≡T₁(t) is how the measured T₁ value at time t changes due to the concentration of contrast agent according to:

$$1 / {T}_{1} ( t ) = {r}_{1} {C}_{t} ( t ) + 1 / {T}_{10} , \qquad ( 8 )$$

where r₁ is the relaxivity constant specific to the contrast agent, and T₁₀ is the native T₁ (from the pre-contrast T₁ map obtained from step 21). Finally, Cₜ(t) is described using the BAT-shifted AIF (i.e., Cₚ(t) from Eq. (7)) and fitting each voxel's concentration time course to the extended Kety-Tofts model,

$${C}_{t} ( \overline{x} , t ) = {K}^{t r a n s} ( \overline{x} ) {\int }_{0}^{t} {C}_{p} ( u ) exp ( - ( {K}^{t r a n s} ( \overline{x} ) / {v}_{e} ( \overline{x} ) ) ( t - u ) ) du + {v}_{p} ( \overline{x} ) {C}_{p} ( t ) , \qquad ( 9 )$$

where Cₜ(x̅,t) and Cₚ(t) are the contrast agent concentrations in the tissue and plasma, respectively, at position x and time t; K^{trans}(x̅) is the volume transfer constant of contrast agent from the plasma to the tissue space at position x, vₑ(x̅) is the extravascular extracellular volume fraction at position x̅, and vₚ(x̅) is the plasma volume fraction at position x̅. The measured signal intensity time course for each voxel within the tumor ROI can now be fit by this nested set of three equations (i.e., Eq. (9) substituted into Eq. (8), which is then substituted into Eq. (5) and Cₚ(t) from Eq. (7), using lsqcurvefit (MATLAB). This fitting routine provides a value of K^{trans}, vₑ, and vₚ at each voxel position, x̅, within the tumor ROI. Calculate the efflux constant, kₑₚ(x̅) as K^{trans}(x̅)/vₑ(x̅). Save the resulting four pharmacokinetic parameter maps to the .mat file in the MATLAB structure for that patient and visit.

Step (25). Generate enhanced anatomical images. Enhance contrast by applying a local-statistics-based transfer function to each voxel of a subtraction anatomical image (average pre contrast images minus average post contrast images). Specifically, use the transfer function

$${I}_{e n h} = \{ \begin{matrix}{I}_{c} - \sqrt{{I}_{c}^{2} - {I}_{orig}^{2}} , & 0 < X \leq {X}_{c} \\ {I}_{c} - \sqrt{{( {I}_{max} - {I}_{c} )}^{2} - {( {I}_{max} - {I}_{orig} )}^{2}} , & {X}_{c} < X \leq {X}_{max}\end{matrix} \qquad ( 10 )$$

where I_{org} is the original image, Iₘₐₓ the maximum intensity in the original image, and Iₑₙₕ is the resulting enhanced image. This transfer function is S-shaped, so I_{c} is the inflection point defined as the 95th percentile of the intensity. This strategy for histogram normalization ensures that foreground enhancement as well as background suppression can be achieved simultaneously. Save the resulting enhanced anatomical images to the .mat file in the MATLAB structure for that patient and visit.

Mapping the imaging data to the mathematical model, timing˜approximately one day (steps 28-30 are the rate limiting steps as they require the use of all patient data in a study)

The following steps (i.e., steps 26-36) describe methods for converting the processed MRI data into a single modeling domain and deriving relevant, physical quantities for model simulations starting with inter-visit registration.

Step (26). Manually compare the slices across the different visits evaluating each patient's anatomy to determine a rough slice alignment. (We note that this is the first-time data from different visits are compared; i.e., one cannot complete this step or continue to the next steps until data is acquired from at least two visits.) Using enhanced images, utilize unique characteristics in the patient's anatomy (visible structures in the tissue and vessels) and the tumor bearing slices to determine which of the 10 slices for each visit correspond to the slices across all visits. Performing this initial alignment improves the ability of the registration algorithm to converge to a solution. Save the corresponding slices to the .mat file in the MATLAB structure for that patient and visit. This is performed prior to applying the registration algorithm.

Step (27). Convert files to .mhd files. This can be accomplished by using MATLAB's Medical Imaging Toolbox function write_mhd. To use this function, save the tumor ROIs and enhanced anatomical images for the slices determined in step 26 for registration. Additionally, define parameter files for each patient and scan to be registered (further instructions for formatting of these files can be found at http://elastix.bigr.nl/wiki/index.php/Parameter_file_database). Once these files are generated, perform registration by calling the elastix function, which first rigidly aligns the tumor ROIs and corresponding anatomical images followed by the b-spline registration with rigidity penalty over the tumor ROI. Once the deformation field is generated, apply the deformation field to all remaining maps and images for each patient set by calling the transformix function. This step is performed prior to running Elastix.

Step (28). Explore a range of rigidity penalty weights. To select an appropriate weight for the rigidity penalty, apply the above registration functions (step 27) for a range of values for subset of patients, where scan 2 is the “target” image and scan 1 as the image to be registered.

Step (29). Evaluate rigidity penalty weights. Evaluate the results of the different penalty weights (from step 28) using the following metrics:
Metric 1: similarity or normalized mutual information between reference image and registered target image
Metric 2: sum of the Jacobian determinant of the final deformation field within the tumor region
Metric 3: consistency between the distributions of quantitative parameters before and after registration

Select a weight that maximizes metrics 1 and 3 while minimizing metric 2. For metric 3, divide histograms (probability density functions) of the original target and registered maps into the same 100 segments (i.e., converting the histograms into vectors) and then normalize to calculate their inner product as similarity.

Step (30). Select a rigidity penalty weight. Compare the resulting registered images (step 28) to identify a “tolerant range” of the studied configuration parameter (i.e., weight of the rigidity penalty term). Within this tolerant range, the performance of registration is reasonable and similar; outside the range, the registration either fails or significantly changes values within the tumor ROI (per the metrics described in step 29). Note that the tolerant range varies between different patients, as well as between different visits for individual patients. Therefore, choose a value that falls in the tolerant range for all the patients used in this parameter investigation.

Step (31). Register the images. Using the details of step 27 and the penalty weight derived from step 28-30, register the enhanced images of scans 1, 3, and 4 to scan 2 (target). Apply the resulting deformation fields to all corresponding patient parameter maps. Save the resulting registered data to new .mat files (separate from previous data analysis files) in the MATLAB structure for that patient and visit.

The following steps describe defining and calculating modeling quantities.

Step (32). Define modeling domains. Manually draw breast ROIs to define the domain for mathematical modeling. Using the enhanced images, for each slice, carefully outline the breast region outside of the chest wall and within the skin of the organ using the MATLAB function roipoly. Save the outlined shapes as binary masks, with zeros outside the breast ROI. If the tumor is not near the chest wall, segmentation can simply eliminate the chest region. Save the resulting breast masks to the .mat file in the MATLAB structure for that patient and visit.

Step (33). Calculate tumor cells per voxel. Using the MATLAB function imfilter, smooth the registered ADC maps for each slice using a Gaussian filter with size 3×3 voxels. Convert the ADC value for each voxel within the tumor (as segmented using the above methods) to an estimate of the number of tumor cells per voxel at each position x̅ and time t, N_{TC}(x̅,t), via established methods:

$${N}_{T C} ( \overline{x} , t ) = θ ( \frac{{ADC}_{w} - ADC ( \overline{x} , t )}{{ADC}_{w} - {ADC}_{min}} ) , \qquad ( 11 )$$

where ADC_{w} is the ADC of free water (3×10⁻³ mm²/s), ADC(x̅,t) is the ADC value for the voxel at position x̅ and time t, and ADCₘᵢₙ is the minimum (positive) ADC value over all tumor voxels for the patient across all scans. The parameter θ is the carrying capacity describing the maximum number of tumor cells that can physically fit within a voxel; its numerical value can be determined by assuming a spherical packing density of 0.7405, a nominal tumor cell radius of 10 μm, and the voxel volume of 8.45 mm³ (using the DCE-MRI resolution). Save the resulting tumor cell map (N_{TC}(x̅,t) to the .mat file in the MATLAB structure for that patient and visit.

Step (34). Segment the fibroglandular and adipose tissues. Use the enhanced imaging data and a two-class k-means clustering (MATLAB function kmeans) to generate initial masks for fibroglandular and adipose tissues. To suppress noise and eliminate voxels containing both tissues, apply k-means clustering for a second time on the adipose region segmented by the first clustering to erode the edges. Finally, for the fibroglandular tissue mask, eliminate small “islands” (≤10 connected voxels) using MATLAB function bwareaopen. Save the resulting segmentation masks to the .mat file in the MATLAB structure for that patient and visit.

Step (35). Approximate drug delivery. To approximate concentrations of drug delivered throughout the tumor tissue, utilize the derived physiological parameters from the perfusion/diffusion analysis and each patient's individual therapeutic regimen. Using Eq. (9) (the Kety-Tofts model) whereby the DCE-MRI derived parameters (K^{trans}, vₑ, and vₚ) are available for each voxel (from the analysis described in step 24), replace the Cₚ(t) term in Eq. (9) with the concentration of drug in the plasma from measured population curves for each drug the patient received with repeated doses according to each patient's specific therapeutic regimen. Save the resulting drug distributions as a 4D matrix (time being the fourth dimension) to the .mat file in the MATLAB structure for that patient and visit.

Step (36). Calculate tumor summary measures. Calculate the total tumor cellularity by summing the number of tumor cells across all the voxels in the tumor ROI. Approximate the tumor volume as the product of the total number of voxels within the segmented tumor ROI and the voxel volume (8.45 mm³ using the DCE-MRI spatial resolution described above in section 7). To calculate the longest axis of each tumor, evaluate the 3D tumor ROI using the function regionprops3 within MATLAB, which approximates the longest possible axis within a 3D object. These measures of cellularity, volume, and longest axis are to be applied to all of the model's predictions to enable direct comparison to the clinically measured data. Note that by implementing this process of image segmentation and longest diameter calculation makes the evaluation as rigorous as possible.

The following steps provide the methods for implementing a mathematical model to utilize the above patient-specific, MRI-derived quantities to generate individual patient response predictions.

Tumor forecasting, timing˜10 hours per patient, can be up to several days depending on tumor size.

Step (37). Implement the model (i.e., Eqs. (1-3)) in 3D. Use a fully explicit finite difference scheme with central difference in space and forward difference in time. With voxel dimensions defined by the size of the DCE-MRI voxel grid, a maximum diffusion coefficient of 0.001 mm²/day, a time step of Δt=0.25 day will ensure numerical stability. Set the size of the computational domain by a square whose dimensions are determined by the size of the breast domain for each patient. Assign the tissue stiffnesses in Table 1 to the tumor, fibroglandular, and adipose tissue ROIs for the mechanical coupling. Apply a no flux boundary condition for the tumor cells and zero tissue displacement (i.e., \vec{u}=0 at the x-boundary) on the breast domain boundary. For computational efficiency, solve the mechanical coupling (Eq. (3) to the diffusion (Eq. (2) every 20 timesteps. Please also see the troubleshooting table for additional details to ensure simulation accuracy.

Step (38). Calibrate model parameters for each patient. Utilize two of the MRI data sets for each patient to calibrate the mathematical model and then simulate the model to a later scan or the time of surgery to make a prediction of tumor response. For example, the data sets from visits 1 and 2 are used for calibration to the first therapeutic regimen, thereby enabling simulating the model and predicting the measured response of the tumors at the time of visit 3. Similarly, use data sets from visits 3 and 4 for calibration to the second therapeutic regimen to simulate the model and predict the response of tumors at the time of surgery (as determined by pathology). See FIG. 2 for an illustration of this calibration and prediction strategy.

Use the cellularity maps of the two scans for each patient (derived from the ADC of the DW-MRI data—see steps 20 and 33) to calibrate model parameters, where the tumor from the earlier imaging visit initializes the calibration (with the corresponding tissue maps for mechanical coupling and distributions of drugs) and the later tumor ROI is the target for calibration. Calibrate the D₀ and α parameters globally, and the k parameter map spatially, where the remaining parameters are assigned to the literature values in Table 1 and θ is directly calculated (step 33). Utilize a Levenberg-Marquardt least squares, non-linear optimization for calibration, where the sum of squared errors between the simulated tumor cell numbers from the model and the calculated number of tumor cells from the imaging data is minimized with the following parameters: maximum number of iterations=200, initial lambda=10⁻²⁰, lambda increment factors=9 and 11, and perform the Jacobian calculation every 25 iterations. Additionally, bound the global parameters D₀ and α to be greater than zero, and bound D₀<0.001 mm²/day. Stopping criteria are the sum of the squared errors<0.001, concordance correlation coefficient of one between the model simulation and target distribution of tumor cells, and/or after the maximum number of iterations has been realized.

Step (39). Assess uncertainty. For the following predictions and evaluations, uncertainty related to the calibrated parameters and data measurements can be considered. Choose three representative data sets from the cohort. Using the calibrated parameters, simulate the model from scan 1 to scan 2 (target scan for calibration) for each tumor. Add an appropriate range of noise (determined by, for example, repeatability studies for DW-MRI data) to voxels in each tumor cell map of the model generated scan 2 results using a normal distribution (MATLAB function randn). Calibrate the model to the noisy scan 2 tumor cell map and save the resulting parameter values. Repeat for a total of 100 sets for each tumor in the three representative data sets (N=300 total). Calculate the percent difference for each parameter between the corresponding original parameter values and each of the results from fitting the noisy data. Calculate the 95% confidence interval for each parameter using all samples. Determine whether a uniform or normal distribution is representative of the resulting parameter difference distributions.

For each patient in the cohort, generate 50 random parameter sets by sampling distributions of the parameters defined by the calculated 95% confidence intervals centered about each patient's calibrated parameter set (use MATLAB's rand for uniform distributions or randn for normally distributed values). Simulate the model from scan 2 to scan 3 for each of the 50 randomized parameter sets for each patient. Calculate the 95% confidence interval across all 50 resulting tumor predictions for each patient for total cellularity, volume, and longest axis. Therefore, simulation results will include confidence intervals based on the uncertainty in the parameter estimates.

Step (40). Forecast tumor response. Using the corresponding calibrated parameters and maps (tissues, tumor cellularity, drug distributions) from scan 2, simulate the model from the time of scan 2 to scan 3. Evaluate the resulting 3D prediction with measured tumor response using the summary measures (total tumor cellularity, total tumor volume, and longest axis, step 36). Additionally, compare the predicted tumor and measured scan 3 tumor with the Dice coefficient (measuring overlap of the predicted ROI and measured ROI; a Dice of zero indicates no overlap, whereas a Dice of 1 indicates perfect overlap), and/or the concordance correlation coefficient to directly compare the prediction to the measurement for each patient. The predicted percent change from baseline to scan 3 can be compared to actual response defined by RECIST.

Using the corresponding calibrated parameters and maps (tissue, cellularity, drug distribution) from scan 4, simulate the model from the time of scan 4 to scan surgery. Compare the resulting summary measures and their corresponding predicted percent changes to surgically defined responses for the cohort (e.g., pCR vs non-pCR groups) using appropriate statistical comparison test for the cohort size (i.e., t tests, Wilcoxon rank sum test, Kendall and Pearson correlation coefficients etc.). See Table 3 for troubleshooting guidance.

Timing: Here is provided a detailed breakdown of timing for each individual step in the protocol; all times are approximate.

Step 1—highly variable depending on local method of accruing patients; Step 2—15 min to consent a patient; Step 3—10 min to place the IV line; Step 4—2 min to determine the FOV for the MRI exam; Step 5—1 min and 39 sec to acquire the DW-MRI data; Step 6—34 sec to acquire the B1 map data; Step 7—3 min 13 sec for the pre-contrast high-resolution T1-weighted scan, and 99 sec for the variable flip angle T1-weighted images; Step 8—8 min to acquire the DCE-MRI data; Step 9—3 min 13 sec for the pre-contrast high-resolution T1-weighted scan; Step 10—5 to 10 min to upload to PACS; Step 11—6 min for DICOM reading and conversion; Step 12—1 min to check the slice positions using the DICOM header information; Step 13—3-4 min to upsample the DW-MRI and B1 map data to the resolution of the DCE-MRI data; Step 14—1 min to align the DW-MRI data to the DCE-MRI data; Step 15—7 min to align the variable flip angle T1-weighted images to the DCE-MRI data; Step 16—1 min to align the B1 data to the DCE-MRI data; Step 17—9 min to correct for motion within the DCE-MRI data; Step 18—20 min to segment the tumor; Step 19—5 min to perform quality control of the acquired data; Step 20—4 min to calculate the ADC maps; Step 21—22 min to compute the B1-corrected T1 maps; Step 22—5 min to construct a patient's AIF; Step 23—5 min to determine the BAT for each voxel in the DCE-MRI data set; Step 24—25 min to perform the pharmacokinetic analysis for each voxel in the DCE-MRI data; Step 25—2 min to enhance the anatomical images; Step 26—10 min to compare slices across all visits for each patient; Step 27—1 min to convert filetypes to .mhd; Steps 28—30-1 day to identify longitudinal registration weighting for all patient data from all patient visits; Step 31—15 min to align parameter maps from all visits for each patient; Step 32—5-10 min to select domain for mathematical modeling for each patient set (i.e., all scan sessions from one patient); Step 33—1 min to convert ADC maps to estimates of cell number; Step 34—1 min to segment to the breast tissue into fibroglandular and adipose tissues; Step 35—1 min to estimate spatiotemporal distribution of drug concentration; Step 36—1 min to calculate the estimate of tumor cells, total tumor volume, and longest axis; Step 37—5 minutes to simulate the model forward in space and time; Step 38—several hours to 2 days to calibrate the model to the patient data depending on the domain size; Step 39—one day to assess uncertainty in model predictions; Step 40—5 min to evaluate predictive ability.

### Results

In the following sections, we describe and present illustrative results associated with processing one data set through the entire protocol. The example data set used is that of a breast cancer patient with triple negative (estrogen receptor, progesterone receptor, and human epidermal growth factor receptor 2 negative) invasive ductal carcinoma in the left breast. At the time of the first imaging session, the patient was 59 years of age with a body mass index of 28.3. All four scans were acquired over a period of 6 months during which the patient was treated with NAT. Surgery determined that the patient had residual disease (i.e., a non-pCR outcome) at the conclusion of NAT.

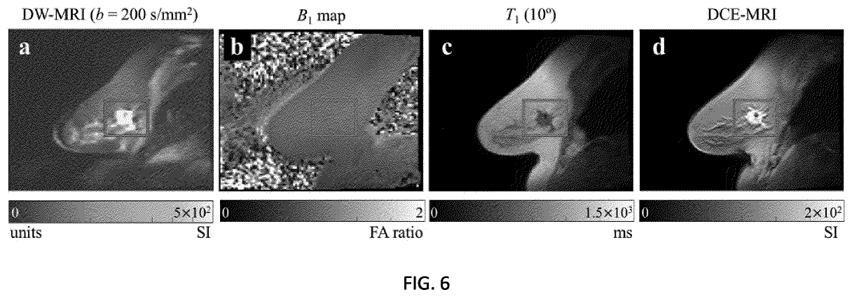

Image acquisition: See FIG. 6 for examples of the resulting MRI data collected for the patient's first scan from the data acquisition steps. Panel a shows the lower b-value diffusion weighted image (see step 5), while panel b presents the B₁ map quantifying the difference between the prescribed flip angle and that actually experience by each voxel (step 6). Panel c presents a single 10° flip angle image from the multi-flip angle data acquired to estimate the T₁ map (step 7), and panel d displays the average of all images acquires during the DCE-MRI sequence (step 8). Note the differences between the various MRI modalities; in particular, observe how the DCE-MRI data (panel d) allows for visualization of the tumor and other tissue structures.

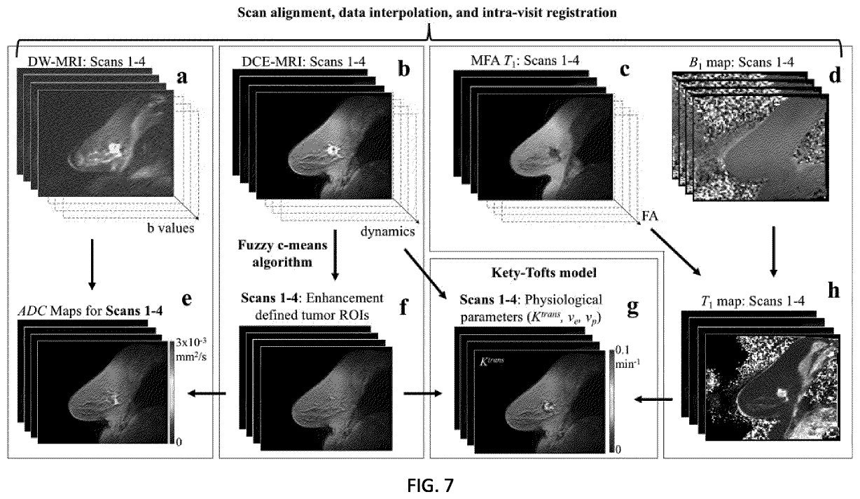

Data processing and analysis: See FIG. 7 for how the example images in FIG. 6 are processed using the protocol (steps 10-25) to identify various properties of the patient's tumor. In particular, the ADC map is derived from the DW-MRI data acquisitions (step 20); the tumor ROI is defined using the DCE-MRI data and FCM algorithm (step 18), a corrected T₁ map is generated from the variable flip angle T₁ images and the B₁ map (step 21), and the resulting Kety-Tofts parameters characterizing the vasculature within the tumor are derived utilizing the DCE-MRI data, tumor ROI, and corrected T₁ map (step 23).

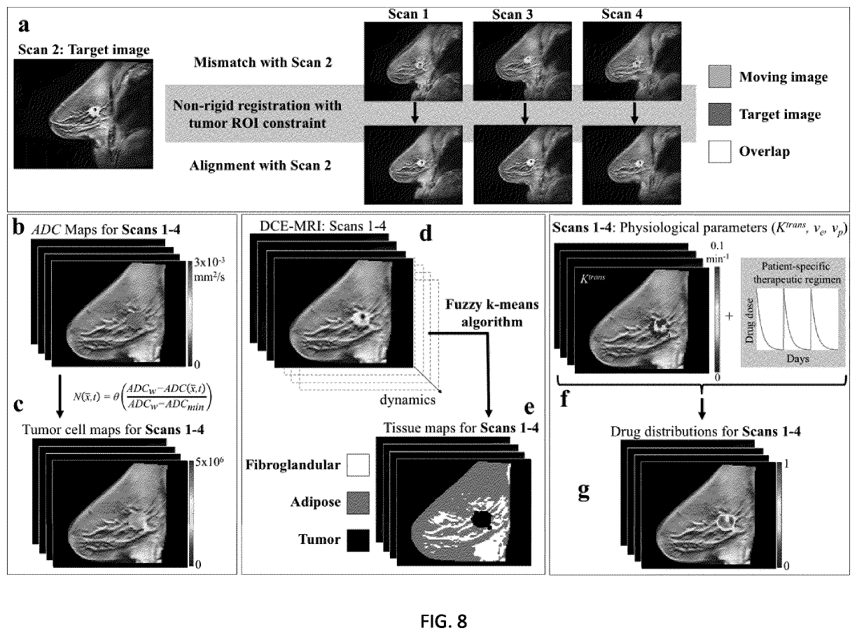

Mapping imaging data to the mathematical model: See FIG. 8 for example intermediary and subsequent images for the steps that convert the quantitative data maps to quantities utilized within the mathematical modeling system. Here for example images for all four scans can be visualized in the inter-visit registration panel a (steps 26-31). After inter-visit registration, a modeling domain is identified over the breast (step 32), ADC values within the tumor ROI are converted to tumor cellularity (step 33), masks for the fibroglandular and adipose tissues are generated using a k-means clustering algorithm (step 34), and drug distribution in the patient's tissue is approximated using the Kety-Tofts model and plasma concentration curves of the patient's therapeutic regimen (step 35). For this patient, the resulting summary measures described in step 36 for visits 1-4 are: total cellularity (cells) of 1.53×10⁹, 1.42×10⁹, 1.13×10⁹, and 1.20×10⁹; volumes (mm³) of 14,462, 11,831, 5,813, and 4,551; longest axes (mm) of 36, 34, 32, and 29, respectively.

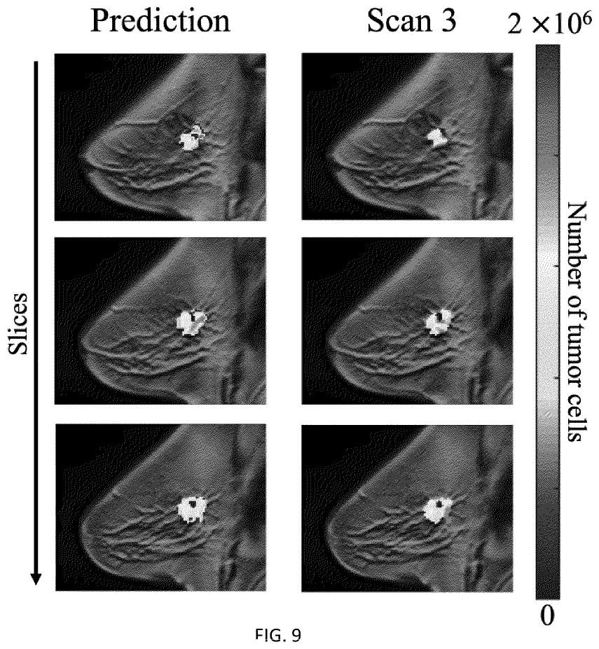

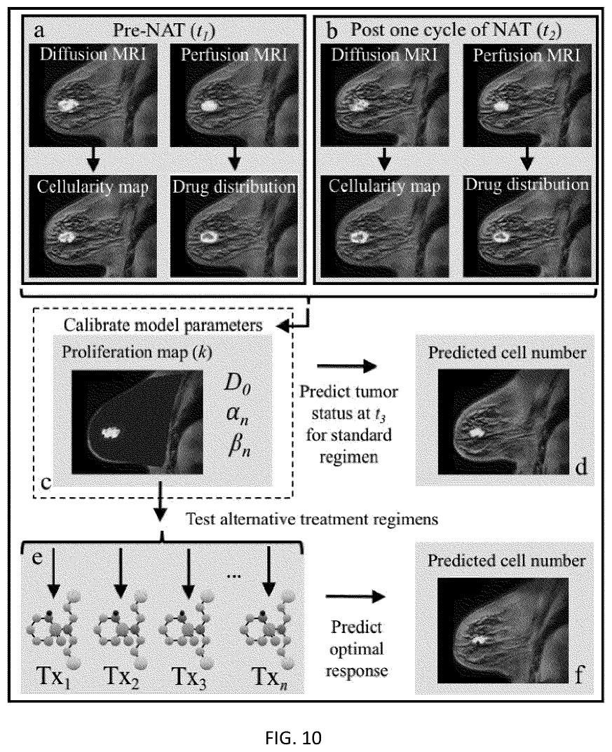

Tumor forecasting: See FIG. 9 for an example comparison of the prediction from the mathematical model and the experimentally measured data for three central slices at the time of scan 3 for the same patient data presented in FIGS. 6-8. Specifically, the mathematical model was calibrated using the patient's data from her first two scans, and then, with the resulting patient-specific parameters, the model was simulated forward in time from scan 2 to the time of scan 3 (as described in steps 37-40). Note that the model is able to predict the patient's tumor response with errors<17% for the three summary tumor measures (total cellularity, volume, and longest axis) and has strong statistical correlation with the shape and cellular densities of the tumor (see figure caption for more details).

TABLE 1
Description of variables for the model system.

| Variable | Description | |
|---|---|---|
| N_{TC}(x̅, t) | Number of tumor cells in the voxel at position x̅ at time t | |
| D | Diffusion coefficient of tumor cells, where | |
| | D = D₀exp(−γσ_{νm}(x̅, t)) (mm²/day) | |
| σ_{νm} | Von Mises stress (N/m²) | |
| [symbol] | Displacement vector due to tumor cell growth (mm) | |
| G | Shear modulus due to breast tissue properties, | |
| | where G = E/(2(1 − ν)) (kPa) | |
| Cₜᵢₛₛᵤₑ^{drug} | Concentration of drug in the tissue in the | |
| (x̅, t) | voxel at position x̅ at time t (μM) | |
| Parameter | Description | Value |
| D₀ | Diffusion coefficient of | Calibrated, mm²/day |
| | tumor cells without stress | |
| γ | Mechanical coupling | Assigned at 2.0 × 10⁻³ |
| | coefficient for stress | (m²/N) |
| ν | Poisson's ratio | Assigned, 0.45 |
| | | (dimensionless) |
| E | Young's modulus for adipose, | Assigned, 2 kPa, 4 kPa, |
| | fibroglandular, and tumor tissues | and 20 kPa, respectively |
| λ | Coupling constant for | Assigned as 2.5 × 10⁻³ |
| | displacement of tumor cells | (N · m/cell) |
| k(x̅) | Proliferation rate of tumor | Calibrated, 1/day |
| | cells in the voxel at position x̅ | |
| θ | Carrying capacity of tumor | Defined, cells |
| | cells per voxel | |
| α | Efficacy of the drug against | Calibrated, 1/(μM · day) |
| | tumor cells | |

TABLE 2
Breast MRI acquisition parameters. All breast MRI data were acquired in the
sagittal plane with a field of view of 256 × 256 mm².

| | | | High- | T₁ mapping: | |
|---|---|---|---|---|---|
| | | | resolution T₁ | variable | |
| MRI | | | (pre- and | flip | |
| parameters | DW-MRI | B₁ mapping* | post-contrast) | angle | DCE-MRI |
| Scan sequence | single-shot | TurboFLASH | T₁-weighted, | 3D gradient- | T₁-weighted, |
| | spin echo, | | VIBE | echo FLASH | VIBE |
| | echo planar | | | | |
| TR (ms) | 3000 | 8680 | 5.3 | 7.9^{‡} | 7.02 |
| TE (ms) | 52 | 2 | 2.3 | 2.4^{‡} | 4.6 |
| Flip angle | 90 | 8 | 10 | 2-20 | 15 |
| (degrees) | | | | | |
| Acquisition | 128 × 128 | 96 × 96 | 256 × 256 | 192 × 192 | 192 × 192 |
| matrix | | | | | |
| Slice | 5^{†} | 5^{†} | 1 | 5^{†} | 5^{†} |
| thickness (mm) | | | | | |
| GRAPPA | 2 | None | 2 | 3 | 2 |
| acceleration | | | | | |
| factor | | | | | |
| Fat suppression | SPAIR | None | SPAIR | None | None |

*Due to the inclusion of a slice gap in the B1 mapping protocol, two acquisitions (at 17 seconds each) were performed to cover the same field-of-view as the other measurements.
^{‡}The parameters should be set to “minimum” given the confines of the other pulse sequence settings.
^{†}If adequate signal-to-noise can be maintained, then these values can be reduced.

TABLE 3
Troubleshooting table

| | | | |
|---|---|---|---|
| 4 | Unable to identify | Breast tissue anatomy and tumor | Biopsy clips and radiological notes |
| | the tumor | sizes/shapes vary widely between | are frequently helpful for |
| | | patients and it may be very | identifying the tumor location. |
| | | difficult to identify the tumor. | Biopsy clips create a dark signal |
| | | | and are easily spotted. |
| 5 | Low SNR | The resolution of the image may | Boost SNR by choosing a lower |
| | | be too fine. | resolution or re-gridding the image |
| | | | post acquisition |
| 6 | When a product | Scanner is not equipped with a | After an initial uncorrected T₁ map |
| | sequence for | B₁-mapping sequence | is made using the variable-flip |
| | measuring B₁ is | | angle sequence approach, use |
| | not available | | threshold to select only those |
| | | | voxels that most likely belong to |
| | | | adipose fat. Based on the |
| | | | assumption that T₁ values should |
| | | | be a consistent value for these |
| | | | voxels, compute an estimate of the |
| | | | flip angle error by assuming the |
| | | | acquired map includes B₁ error. |
| | | | Once the B₁ map is filled in for |
| | | | adipose fat voxels, use spatial |
| | | | interpolation to estimate B₁ at |
| | | | other (non-fat) voxels. This |
| | | | correction, while suboptimal to |
| | | | performing a true B₁ ap, should |
| | | | serve to reduce the magnitude of |
| | | | flip angle error when B₁ mapping |
| | | | sequences are not available on the |
| | | | study scanner. |
| 12 | A certain scan | This often happens during | Only utilize the aligned slices or |
| | acquisition | scanning for variable flip angle | do a 3D interpolation to re-align |
| | contains more | T₁ mapping data, the high | the images for downstream |
| | slices than | resolution T₁-weighted data | analysis and save the data in a |
| | included for the | | new structure |
| | DCE-MRI data | | |
| 18 | Too much or too | FCM algorithms have a threshold | Adjust the membership threshold |
| | little tumor results | that can be tuned for belonging | in the FCM algorithm. |
| | from segmentation | to different partitions. | |
| 20 | Unable to derive | Using low b-values (less than | Including more than two b-values |
| | expected values | 100 s/mm²) can be affected by | can improve the accuracy of |
| | for ADC | tissue perfusion thus, altering the | parameter estimation. |
| | | interpretation of the image. | |
| | | Poor data fitting | May need to use interpolation |
| | | | methods to populate ADC values |
| | | | for voxels with bad fits. |
| 21 | Signal drop in the | Standard-of-care exams may | May need to assume an analytical |
| | AIF | administer high concentrations | form of the concentration of the |
| | | of contrast agent, which may | contrast agent in the plasma and |
| | | induce T2* effects in the signal | fit to the average signal intensity |
| | | intensity curves measured from | time-source. |
| | | the large vessels (i.e., those | |
| | | typically employed for AIF | |
| | | estimation). | Bound calibration method or |
| 22 | Unphysiological | Could be in regions of necrosis | filter out values beyond the |
| | values from the | where the Kety-Tofts model does | physiological range from |
| | pharmacokinetic | not account for such phenomena | downstream analysis (may need |
| | moodel fits | | to use interpolation methods to |
| | K^{trans}, νₑ, νₚ, | | populate filtered out voxels). |
| | kₑₚ < 0, νₑ, | | |
| | νₚ > 1, and/or | | |
| | K^{trans}, kₑₚ > 5 | | |
| 26 | Alignment of | Can occur despite best efforts to | Additional blank slices may be |
| | tumors/anatomy | center the FOV on the center of | needed to “pad” the ends of any |
| | is off by several | the tumor during scanning-due to | particular scan set to achieve |
| | slices | the tumor dramatically changing | alignment and maintain equal |
| | | in size and shape during the | dimensions across data sets. |
| | | course of therapy | |
| 31 | Inter-visit | Despite careful initial slignment | Additional manipulations may be |
| | registration failure | of slices and selection of the | required including trimming the |
| | | tumor ROI weight factor, the | moving images to remove large |
| | | registration can fail (especially | amounts of image outside the |
| | | for tumors that have greatly | breast and/or remove large |
| | | reduced in size by the time of | amounts of the image that includes |
| | | scan 4). | the chest cavity. Even with these |
| | | | additional corrections, registration |
| | | | can fail or simply be unable to align |
| | | | tumor ROIs. In these cases, simple |
| | | | rigid registration or manual |
| | | | alignment may be necessary. The |
| | | | MATLAB functions imregtform |
| | | | and imwarp can be used for rigid |
| | | | registration. |
| 32 | Unusual breast | Some breasts contain implants | For breasts containing implants, |
| | domain | near the tumor region of interest | two domains can be drawn. The |
| | | that would not be a part of the | first containing the entire breast |
| | | modeling domain | regio and the second where the |
| | | | breast implant is segmented out. |
| | | | The first breast mask can be used |
| | | | for image/display purposes, but the |
| | | | mathematical model simulates on the |
| | | | second domain as we assume the |
| | | | tumor cannot grow into the breast |
| | | | inplant. |
| 35 | DCI-MRI data | We note that certain MRI | In these cases, to approximate a |
| | does not have the | protocols do not allow for the | normalized drug distribution in each |
| | requisite temporal | requisite temporal resolution for | voxel of tissue, a map of the blood |
| | resolution for | pharmacokinetic analysis. | volume can be calculated by |
| | pharmacokinetic | | computing the area under the dynamic |
| | analysis | | curve (AUC) of the baseline- |
| | | | subtracted time course for each voxel |
| | | | and normalized by the AIF AUC |
| | | | value (as an approximation of blood |
| | | | volume) from contrast-enhanced data. |
| | | | The signal enhancement ratio may |
| | | | also be used in this way to |
| | | | estimate drug distribution. |
| 37 | Numerically | This can be due to several | Check the diffusion coefficient, grid |
| | unstable results | numerical scheme errors | size, and time step selected maintain |
| | | | numerical sttability. Verify boundary |
| | | | conditions where tumor cells are |
| | | | conserved and not lost when |
| | | | proliferation is zero and diffusion is |
| | | | large. |
| 38 | Calibration | Calibration can take >1 day per | To reduce computation time for all |
| | computationally | patient when using a spatitally | calibrations, the voxel matrix within |
| | expensive | defined proliferation map for | the designated domain can be down |
| | | large tumors and domains | sampled. Parallelize codes to further |
| | | | reduce computational cost by |
| | | | utilizing multiple available processors |
| | | | concurrently on a server. Focus |
| | | | calibration on a smaller domain |
| | | | containing the tumor ROI and not |
| | | | over the whole breast. Broyden’s |
| | | | method can also be used to |
| | | | approximate the update of the |
| | | | Jacobian for each iteration. |
| 38 | Calibration will | Using maps from the previous | Can use the starting number of tumor |
| | not converge | scan may limit the calibration | cells and shape from scan 1 (scan 3) |
| | | scheme to hit the target (e.g., | with the tissue and drug distribution |
| | | using the scan 1 maps to | maps of scan 2 (scan 4) for |
| | | calibrate to scan 2). Different | calibration. The lambda increment |
| | | tumors may be more difficult | factors increased or decreased are |
| | | for the calibration to converge. | often problem-specific and need to be |
| | | | empirically determined to improve |
| | | | convergence. Larger tumors may |
| | | | require more iterations. |

### Models Constrained by MRI Data for Evaluating Patient-Specific Neoadjuvant Regimens

One approach to individualizing mathematical models is to leverage physiological information (acquired in 3D and at multiple time points) from non-invasive imaging data to initialize and constrain model parameters, thereby enabling patient-specific predictions. Imaging-informed, biologically-derived mathematical models can provide accurate predictions for tumor development in, for example, the kidney, brain, lung, and pancreas. Various embodiments employ DW-MRI data to estimate tumor cellularity, mechanical-coupling of the breast tissue properties with tumor growth based on each individual patient's anatomy, and estimates of drug delivery to each voxel via DCE-MRI. Various embodiments incorporate a mathematical description of the decay and efficacy of chemotherapies (calibrated from each patient's data set). Various embodiments enable allow for identifying alternative—individualized—dosing strategies predicted to outperform the therapeutic regimen each patient would otherwise receive as standard-of-care. In various embodiments, the requisite imaging data may be acquired at community-based radiology centers (i.e., not research-oriented academic medical centers) using widely available hardware. Because various embodiments may be used in such facilities—where the majority (85%) of oncology patients receive their care—the disclosed approach dramatically increases the population these technologies may serve in the future.

### Study Data and Methods

Patient population: Quantitative MRI data was acquired in a cohort of 21 patients diagnosed with intermediate to high grade invasive breast cancers, who were eligible for NAT as a component of their clinical care. Participants received their care and imaging in community care settings (i.e., not academic, research-oriented medical centers). Table 4 summarizes the key clinical features of the patient population.

TABLE 4
Clinicopathologic characteristics of the patient cohort.

| | | | | | Receptor status | Therapy | RECIST | RECIST |
|---|---|---|---|---|---|---|---|---|
| | | | | Tumor | (ER, PR, | from scan | by scan | by scan |
| Patient | Age | BMI | Ethnicity | type | HER2) | 1 to 3 | 2 | 3 |
| 1 | 74 | 18.18 | Hispanic | IDC | (−, −, −) | DC | — | — |
| [symbol] | 25 | 22.46 | Hispanic | IDC | (−, −, −) | DC | SD | SD |
| | 42 | 24.80 | Hispanic | IDC | (+, −, −) | DC | SD | SD |
| | 47 | 24.80 | Asian | IDC | (+, −, −) | DC | SD | CR |
| | 54 | 24.36 | Caucasian | ILC | (+, +, −) | DC | PD | SD |
| 6 | 59 | 28.30 | Caucasian | IDC | (−, −, −) | PC + I | SD | SD |
| 7 | 63 | 18.95 | Caucasian | IDC | (+, +, −) | DC | — | — |
| [symbol] | 27 | 18.79 | Caucasian | IDC | (+, +, −) | DC | SD | PR |
| 9 | 32 | 35.8 | Asian | IDC | (+, +, +) | DC* + T | SD | SD |
| [symbol] | 52 | 21.10 | Caucasian | IDC | (−, −, −) | PC | SD | PR |
| | 38 | 27.50 | Caucasian | IDC | (−, −, −) | PC | SD | PR |
| 12 | 38 | 31.40 | Caucasian | IDC | (−, −, −) | PC + I | PR | PD |
| [symbol] | 62 | 39.32 | Caucasian | IDC | (−, −, −) | DC | SD | SD |
| 14 | 38 | 36.18 | African | IDC | (+, +, +) | DC*+ T | PR | PR |
| | | | American | | | | | |
| [symbol] | 42 | 29.95 | Caucasian | IDC | (+, +, −) | DC | SD | SD |
| 16 | 53 | 33.94 | Caucasian | IDC | (+, +, −) | DC | SD | — |
| 17 | 58 | 31.60 | Caucasian | IDC | (−, −, −) | PC + I | PR | PR |
| [symbol] | 48 | 16.90 | Caucasian | IDC | (+, +, −) | DC | SD | PR |
| | 54 | 26.62 | Caucasian | IDC | (+, +, −) | DC | SD | SD |
| | 41 | 24.21 | Hispanic | IDC | (+, +, +) | DC | PD | PR |
| | 26 | 29.26 | Hispanic | IDC | (−, +, +) | DC | SD | SD |

BMI: body mass index;
IDC: invasive ductal carcinoma;
ILC invasive lobular carcinoma;
ER: estrogen receptor;
PR: progesterone receptor;
HER2: human epidermal growth factor receptor 2.
DC: doxorubicin and cyclophosphamide;
P: paclitaxel;
+T: target therapies trastuzumab and pertuzumab;
PC: paclitaxel and carboplatin;
+I: immunotherapy regimen (pembrolizumab or placebo):
DC*: docetaxel and carboplatin;
CR: complete response;
PR: partial response;
SD: stable disease;
PD: progressive disease.
Bolded labels denote changes in the RECIST category from the scan 1 to scan 2 evaluation to the scan 1 to scan 3 evaluation. Patients 6, 9, and 14 did not receive a second regimen; therefore, for patients 6 and 9, scans 2-4 were aquired after the first through third cycles, respectively, and for patient 14, scan 2 was acquired after the first cycle, scan 3 after the fourth, and scan 4 after the fifth. Three patients (6, 12, and 17

For the majority of the patients, NAT consisted of two regimens; for example, patient 1 received doxorubicin and cyclophosphamide (regimen 1), followed by paclitaxel (regimen 2). MRI data were acquired at four times throughout NAT: 1) prior to therapy, 2) after one cycle of the first therapeutic regimen, 3) at the completion of the first therapeutic regimen, and 4) after one cycle of the second therapeutic regimen. For this study, we utilize the first three data sets summarizing the first regimen. NAT regimens include cycles of doxorubicin and cyclophosphamide approximately every 2 weeks for 4 cycles, paclitaxel (with or without carboplatin and/or targeted therapies) weekly for 3 weeks for 4 cycles (12 total doses), and docetaxel (with carboplatin and targeted therapies) every 3 weeks for 6 cycles. There were some variations in regimens indicated by the treating physician, as is commonly done in the standard-of-care setting (variations reported in Table 4). Note that 14 patients received only cytotoxic therapies for their first NAT regimen, which we refer to as the “chemo” group; while seven patients received additional therapies (targeted or immunotherapies), which we refer to as the “chemo+” group.

MRI data acquisition: MRI was performed at two community imaging facilities. The MRI technologists at each site were directly involved and responsible for positioning the patients and deploying the research imaging protocols. Thus, the image acquisition protocol is designed for practical use for routine imaging using widely available hardware and expertise.

Five MRI data types were acquired at each scan session: 1) a pre-contrast T₁ map, 2) a pre-contrast, B₁ field map to correct for radiofrequency (RF) inhomogeneity, 3) DW-MRI data 4) high-temporal resolution, T₁-weighted DCE-MRI data before, during, and after the injection of a gadolinium-based contrast agent (Gadovist, Bayer, Ontario, Canada, or Multihance, Bracco Diagnostics, New Jersey, USA), and 5) a high-resolution, T₁-weighted anatomical scan (post contrast). The Supplemental Materials provide a more detailed summary of the acquisition parameters for each of these measurements.

Data analysis: Various embodiments of data-processing methods are briefly summarized here (additional details are provided in the below Supplemental Materials section). The first step includes intrascan registration of the MRI data within each scan session to correct for motion via rigid registration. Tumor regions of interest (ROIs) are then identified based on postcontrast scans, and then estimations of tissue properties related to perfusion-permeability of the vasculature are quantified by analyzing the DCE-MRI data with the standard Kety-Tofts model. The DW-MRI data is analyzed to return maps of the apparent diffusion coefficient (ADC) of water. The third step is the interscan registration that aligns the images and calculated maps across all of the patient's imaging sessions into a common domain. The final step calculates the specific quantities to be used within the mathematical model. These quantities include approximating the number of tumor cells from the voxel-based ADC values and segmenting the fibroglandular and adipose tissues (based on enhancement in the DCE-MRI data). To approximate the drug distribution in each voxel of tissue, a normalized map of the blood volume is calculated and then scaled by the peak concentration of drug (as estimated from the Kety-Tofts model; see Supplemental Materials below) to define the initial drug distribution throughout the domain at the time of each dose of therapy.

The volume and longest axis of the tumors from each patient's scans are automatically calculated on the interscan registered images, and the response evaluation criteria in solid tumors (RECIST) is used to assign the response of each patient. We note that we implement this process of image segmentation and longest diameter calculation to make the RECIST evaluation as rigorous as possible so as not to place it at a systematic disadvantage for the comparisons to the mathematical modeling. We note that when computing the longest axis of a tumor, we only consider the central, bulk tumor and disregard smaller, disconnected sections. Using the RECIST evaluation, responders were patients with a complete or partial response (CR and PR, respectively), and nonresponders were patients with stable disease or progressive disease (SD and PD, respectively) comparing tumor burdens in scans 1 and 3.

Model: In various embodiments, a 3D mathematical model that includes the mechanical coupling of tissue properties to tumor growth and the delivery of therapy may be used. This model may be initialized with patient-specific, quantitative, MRI data to predict therapy response. This approach may be extended to account for multiple chemotherapy regimens. The governing equation for the spatiotemporal evolution of tumor cells (NTC) is:

$$\frac{\partial {N}_{T C} ( \overline{x} , t )}{\partial t} = {\overbrace{∇ \cdot ( D ( \overline{x} , t ) ∇ {N}_{T C} ( \overline{x} , t ) )}}^{diffusion} + {\overbrace{k ( \overline{x} ) ( 1 - {N}_{T C} ( \overline{x} , t ) / θ ) {N}_{T C} ( \overline{x} , t )}}^{growth} - {\overbrace{{C}_{drug} ( \overline{x} , t ) {N}_{T C} ( \overline{x} , t )}}^{therapy} \qquad ( 12 )$$

where the first term on the right-hand side describes tumor cell movement (diffusion, D), the second term describes the logistic growth of the cells with carrying capacity θ and proliferation rate k per voxel, and the third term describes the effect of chemotherapy where C_{drug}(x̅,t) is the concentration of the drugs in the tissue. (See Supplemental Materials section below for additional modeling details.) The first term on the right-hand side of Eq. (12), representing the random diffusion (movement) of the tumor cells (D(x̅,t), is mechanically linked to the breast tissue's material properties via the von Mises stress. Additionally, we enforce equilibrium between the tumor and its environment dependent upon changes in tumor cell number. Therefore, the diffusion term encompasses tumor changes (growth or response to therapy) that can cause deformations in the surrounding healthy tissues (fibroglandular and adipose tissues), potentially increasing stress and therefore reducing the outward expansion of the tumor. The carrying capacity is defined as the maximum number of tumors cells that can physically fit within a voxel, while the proliferation rate is calibrated per voxel for each individual patient.

The therapy term describes the spatiotemporal distribution of each drug in the tissue and its effect on the cells of each voxel. Here we expand the model to acknowledge their differing efficacies and decay rates using the following equation:

$${C}_{drug} ( \overline{x} , t ) = {\overbrace{{α}_{1} {C}_{{drug}_{1}} ( \overline{x} , {t}^{*} ) exp ( - {β}_{1} t )}}^{chemotherapy 1} + {\overbrace{{α}_{2} {C}_{{drug}_{2}} ( \overline{x} , {t}^{*} ) exp ( - {β}_{2} t )}}^{chemotherapy 2} , \qquad ( 13 )$$

where αᵢ is the efficacy of each drug, C_{drug}_{ᵢ}(x̅,t*) is the initial concentration of each drug for each dose with t* being time relative to the patient scan data (described in more detail below), and the exponential decay terms (exp(−βᵢt)) represent the eventual washout of the drug over time after each dose. The αᵢ and βᵢ parameters are calibrated for each patient and each drug, where the βᵢ calibration is restricted using bounds defined from ranges in the literature for the terminal elimination half-lives of each drug. The initial concentration of drug, C_{drug}_{ᵢ}(x̅,t*), is approximated using the DCE-MRI data as described above (also see Supplemental Materials below). This concentration is dependent on the time t*, indicating that for the calibration of the model the drug distribution map may be derived from scan 1, but an updated drug distribution map from scan 2 is provided to the model to predict the tumor at the time of scan 3. Thus, the concentration of drug in the tumor tissue is spatially non-uniform and temporally varying based on the individual patient's response to therapy and NAT schedule.

Model parametrization and evaluation of predictive ability: Embodiments of the disclosed approach use the first two MRI data sets for each patient to calibrate the mathematical model and the third MRI data set to evaluate the ability of the mathematical model to predict tumor response to therapy. Specifically, various embodiments use the cellularity maps of each tumor from before and after the first cycle of NAT (scans 1 and 2, respectively) to calibrate model parameters (D₀, α, and β parameters are global; k is spatially determined). Using these calibrated, patient-specific parameters, the model is reinitialized with the tumor cellularity, tissue, and drug distribution maps from scan 2 and run forward to the time of scan 3 to predict tumor response. See FIG. 1 for a graphical depiction of this strategy and the Supplemental Materials below for additional details on the numerical implementation.

The predictive ability of the model is evaluated by comparing three measures quantifying tumor response: total tumor cellularity, tumor volume, and longest axis of the tumor. Using these measures, three evaluations are made to assess the model's predictive ability. First, the error between the model's predictions and the patient's actual tumor values (as determined from their corresponding third scan) are calculated for the three measures, as is the significance of the model's predictive accuracy. Second, total cellularity, volume, and longest axis are evaluated across the cohort to determine the level of agreement between the model's predictions and the measured values from the data as a group. Third, the predicted percent change in the three tumor measures from scan 1 to scan 3 are compared between the two RECIST defined responder groups.

Simulating modified therapeutic regimens for the individual patient: Alternative regimens are proposed for the group of patients for which the model's predictions have the greatest correlation with observation (chemo group). The alternative regimens proposed use the same total dose that each individual patient received during their NAT regimen from scan 2 to scan 3, but the alternative regimens vary in the individual doses and frequency between their second and third scans. More specifically, embodiments simulate the effects of alternative regimens that consist of doses that were ½, ⅓, or ¼ of the dose the patients were administered but given 2×, 3×, or 4×, respectively, as frequently as what the patients received. A daily dose fraction was also investigated. For example, if a patient who received their chemotherapies every two weeks, the alternative regimens will be a ½ dose every week, a ⅓ dose every 4-5 days, a ¼ dose every 3-4 days, and a 1/14 dose every day.

Embodiments run the model forward from scan 2 to the time of scan 3 with each patient's previously calibrated parameters and the alternative regimens implemented to evaluate the differences in tumor response across all regimens (see FIG. 1). The model simulation serves as an in silico twin for each patient to determine therapy response for an alternate regimen. This allows us to identify individualized, alternative therapeutic regimens that we hypothesize can lead to greater tumor control when compared to the “one-size-fits-all” approach that is the current standard.

Analysis: To summarize the model's predictive performance, the absolute percent differences between the measured tumor response from the third scan data and the corresponding predicted values from the model are calculated for each patient (median and quartile ranges reported). The accuracy of the patient-specific predictions for total cellularity, volume, and longest axis are tested by generating a Monte Carlo estimated p-value (see Supplemental Materials) to determine the significance of 10, 15, or 20% absolute differences between model predictions and measured outcomes. (The 10-20% range corresponds to measurement precision.) Due to the modest sample size, it is challenging to accurately evaluate the normality of the data. Thus, we calculate Kendall correlation coefficients (KCC) to determine agreement between the model-predicted tumor response and the observed values at the time of scan 3 across the cohort. However, to enable comparison to previous and future efforts with larger data sets, we also report the concordance and Pearson correlation coefficients (CCC and PCC, respectively). The two-sided Wilcoxon rank sum test is used to determine significant differences in the medians of the percent change from scan 1 to scan 3 between the responder versus non-responder groups with p<0.05 considered significant.

To determine which therapeutic regimen yields the greatest tumor control for each patient, embodiments calculate the percent change from initiation of the alternate regimen (scan 2) to the predicted tumor at the time of scan 3. Comparing these percent changes across regimens for each patient, we determine the “optimal” regimen based on the greatest tumor reduction/control. Embodiments then calculate the difference between the percent changes of the standard regimen and the optimal regimen to determine the additional percent tumor reduction potentially achieved if the alternate regimen had been administered. Median and quartile ranges are reported for these values. Using the paired, two-sided Wilcoxon signed rank test, we determine if there is a significant difference between the tumor control achieved by the group of standard regimens compared to the group of alternative regimens by comparing the percent reduction from scan 2 to scan 3 as well as the total tumor burden predicted at the time of scan 3 between the two groups.

Results: Three patients are excluded from the analysis. For patient 1, the tumor was invading the chest wall, which violates the assumption of a no-flux boundary condition employed in the numerical implementation (see Supplemental Materials). For patient 7, a silicon breast implant caused significant artifacts in the DW-MRI data. For patient 16, the third scan was not collected due to scheduling conflicts. Excluding these three data sets reduces the total cohort to N=18 and the chemo subgroup to N=13.

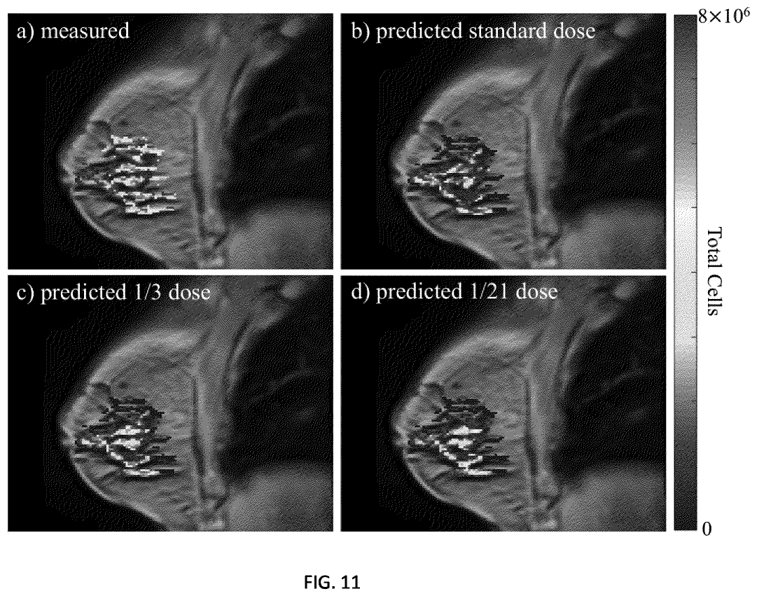

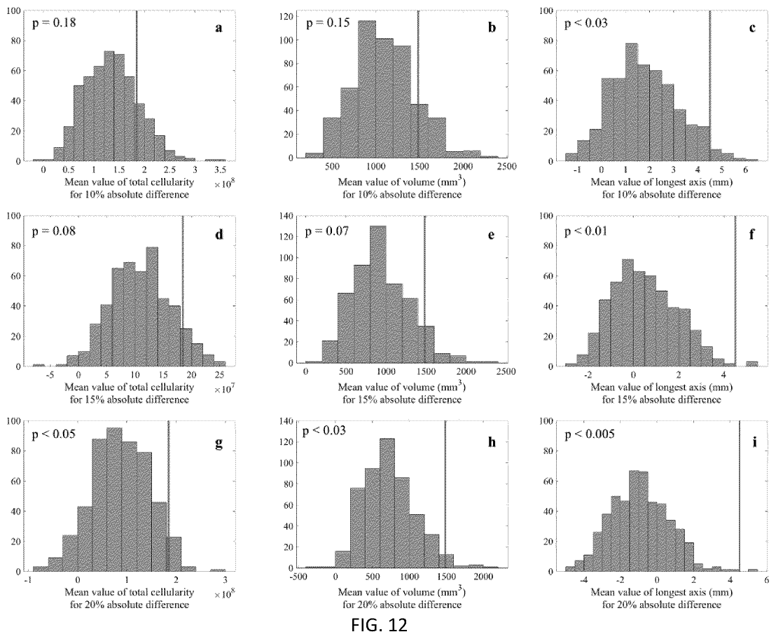

FIG. 11 presents a comparison of the predicted and experimentally measured cellularity maps for a representative patient when the calibrated model is run forward to the time of scan 3 utilizing scan 1 and 2 information. The number of tumor cells predicted by the model are overlaid (in color) on a greyscale anatomical image of the breast. While areas of higher and lower cellularity may not directly match between the prediction and observation, the model is able to capture the general shape of an individual tumor, and there are small errors between the predicted and measured values for the patient's scan (listed in the figure caption). Table 5 summarizes the results for the absolute percent error in all three tumor measures (i.e., total cellularity, total volume, and longest axis) compared to the measured values from each patient's third scan. FIG. 12 depicts distributions of the Monte Carlo generated sample averages for determining the significance of the model's prediction accuracy for 10, 15, and 20% absolute difference between model predictions and measured outcomes. For the cohort (N=18), the model's prediction for longest axis is significant for all three thresholds (p<0.05). The total cellularity and volume measures trend toward significance for the 10% and 15% thresholds (i.e., p<0.1), and achieve p values<0.05 for the 20% threshold.

TABLE 5
Absolute percent errors of model predictions for scan 3 compared to
actual measures derived from each patient's MR images.
Note one patient had no detectable tumor in their scan 3
(N = 17, patient 4 had zero tumor measured by the time of scan 3).

| | Total cellularity | Total volume | Longest axis |
|---|---|---|---|
| Median | 28 | 29 | 14 |
| Quartile range | [8, 95] | [13, 97] | [2, 24] |

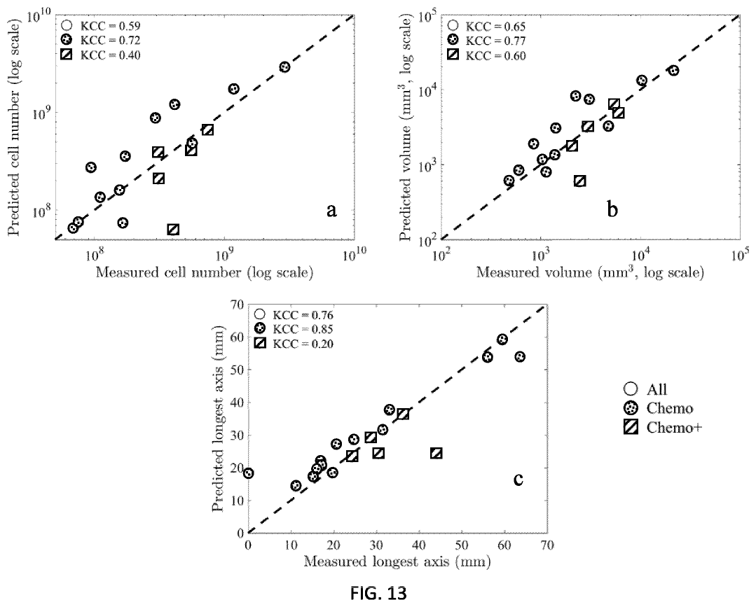

FIG. 13 presents scatter plots comparing the predicted tumor response to the actual tumor response. Across all three measures, the model's predictions strongly correlate with actual tumor response; in particular, we find for cellularity CCC/PCC=0.91/0.92, for volume CCC/PCC=0.90/0.90, and for the longest axis CCC/PCC=0.86/0.88 across the whole cohort (p<0.01). Considering the KCC measure, the predictions are correlated with actual tumor response for these tumor measures: KCC=0.59, 0.65, 0.76 for total cellularity, total volume, and longest axis, respectively (p<0.01). Note that for all three tumor measures, there is greater correlation for the chemo subgroup compared to the chemo+. In particular, the chemo subgroup has greater CCs values than the cohort as a whole where for cellularity we find CCC/PCC=0.92/0.94, for volume CCC/PCC=0.90/0.91, and for the longest axis CCC/PCC=0.92/0.96 (p<0.01). For the KCC measures, the predictions are strongly correlated with actual tumor response for these tumor measures: KCC=0.72, 0.77, 0.85 for total cellularity, total volume, and longest axis, respectively (p<0.01).

The model's predictions are also compared against the tumor response status determined by RECIST at the end of the NAT regimen. Applying RECIST to each tumor from scan 1 to scan 3 results in eight patients labeled as responders and 10 patients as non-responders (see Table 1). See Supplemental Table S.4 for the measured percent changes in longest axis for each of the patients (as well as percent change for total cellularity and volume) from scan 1 to scans 2 and 3. Evaluating the observed percent changes from scan 1 to scan 3, percent change in each of the three tumor measures results in significantly different medians between responder and non-responder groups in total cellularity (p<0.05), total volume (p<0.04), and longest axis (p<0.001). In comparison, the model predicts a significant median percent change between responders and non-responders for the longest axis (p<0.002), while for total cellularity and total volume, p=0.08. See Table 3 for median and quartile ranges for measured and predicted percent change in all three measures. Note that comparing responder and non-responder values for the chemo subgroup (N=6 and N=7, respectively), the model predicts significantly different median changes for all three tumor measures (p<0.04 for total cellularity and volume, p<0.01 for longest axis). Model predicted percent changes for this subgroup are also noted in Table 6.

TABLE 6
Measured and model predicted percent changes from baseline (scan 1) to the completion of the first NAT
regimen (scan 3) for responder and non-responder groups (N = 8 and N = 10, respectively). Note
that a positive percent change indicates tumor regrowth from the baseline. We have noted in parentheses
any differing values for the chemo subgroup (N = 6 and N = 7 for responders and non-responders, respectively).

| | | Total cellularity | | Total volume | | Longest axis | |
|---|---|---|---|---|---|---|---|
| | | | Non- | | Non- | | Non- |
| | | Responders | responders | Responders | responders | Responders | responders |
| Measured | Median | −80 | −50 (−62) | −79 | −60 (−63) | −42 | −12 (−13) |
| Changes | Quartile | [−85 (−88), −56 | [−71 (−75), −11 | [−86 (−90), −63 | [−69 (−74), −22 | [−60 (−64), −37 | [−22 (−23), −2 |
| | range | (−63)] | (−13)] | (−68)] | (−18)] | (−36)] | (−5)] |

| | | |
|---|---|---|
| Total cellularity | Total volume | Longest axis |

| | | | Non- | | Non- | | Non- |
|---|---|---|---|---|---|---|---|
| | | Responders | responders | Responders | responders | Responders | responders |
| Predicted | Median | −69 | 22 (−5) | −65 | −35 (−29) | −30 (−24) | −10 (−6) |
| Changes | Quartile | [−81 (−82), −55] | [−70 (−63), 5 | [−81 (−80), −49 | [−66 (−61), −20] | [−53 (−37), −17 | [−15 (−11), 1] |
| | range | | (10)] | (−50)] | | (−15)] | |

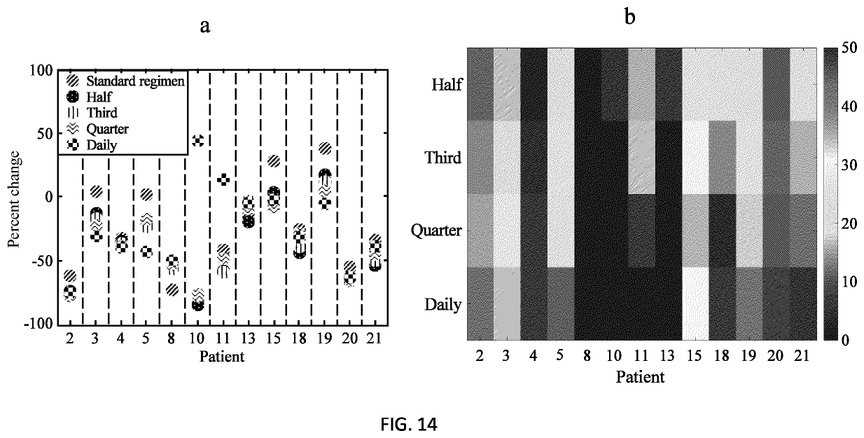

As the model's predictions show the greatest correlation with the chemo subgroup, an in silico study of alternative regimens is performed for this patient subset (see Table 1, N=13)—this choice is further discussed below. Using each patient's parameters calibrated from scans 1 and 2, the model is simulated from scan 2 to the time of scan 3 to determine if greater or lesser control/reduction in the tumor burden can be achieved with the proposed hypothetical alternative therapy schedules (i.e., ½, ⅓, ¼ and a daily dose fraction, given 2×, 3×, 4× as frequently as the standard dose, and daily, respectively). For the two measures pertaining to the size of the tumor (volume and longest axis), comparing their percent change from scan 2 to scan 3 for the standard regimen to that of the alternative regimens tested results in a median difference of 8% for the total volume, and <1% for the longest axis. However, the differences between the standard and the alternative regimens that achieved the greatest reduction in total cellularity per individual patient range from 0% to 46% with a median difference of 17% (quartile range [6%, 35%]). The positive differences represent the additional percent reduction/control that could have been achieved if the patient received the alternative regimen. The 0% instance is the one patient where the standard regimen was optimal amongst all those tested; however, all other patients had additional percent reductions in total cellularity when comparing between the standard and best alternative regimens. FIG. 14 summarizes the predicted percent change from initiation of the alternate regimen (scan 2) to the completion of the therapy (scan 3), as well as a heat map illustrating the differences in the alternative regimens compared to the standard-of-care regimen each patient received. When compared to the standard regimens, the model predicted optimal regimens yield a significantly greater reduction in the total tumor cell number from scan 2 to scan 3 (p<0.001), as well as for the total number of tumor cells predicted at the time of scan 3 (p<0.001). Notice that no single regimen is the most effective across all patients; the daily dosing regimens was best for four patients, while the quarter, third, half, and standard regimens were best for two, two, four, and one patient, respectively. FIG. 2 presents examples of the resulting changes in total cellularity for two alternative regimens for one patient.

### Considerations

In an example embodiment, a mathematical model accounting for patient-specific proliferation and diffusion of tumor cells, mechanical properties of breast tissue, and treatment regimens was individually calibrated with quantitative MRI data acquired in the community-based care setting. After the patient-specific calibration, the model was run forward in time to predict tumor response at the completion of the first therapeutic regimen. Analyzing various absolute differences between model predictions and measured outcomes established significance of the model's prediction accuracy (FIG. 12). The model predictions are also found to be significantly correlated to the actual tumor outcome for the cohort for 3 different measures of tumor response (total cellularity, volume, and longest axis; FIG. 13); in particular, the CCC values for the whole cohort are ≥0.86 when comparing all 3 tumor measurements. Further, the model predictions are significantly different for the change in the longest axis when compared between the RECIST defined responder and nonresponder groups (Table 6). It is important to note that RECIST is only an evaluation of response and is not intended to be employed for predicting response; in fact, the RECIST designation (i.e., CR, PR, SD, PD) from scan 1 and 2 changes for 8 out of the 18 patients when compared to the RECIST designation from scan 1 and 3. However, recall that the mathematical model only required the data from scans 1 and 2 (after just 1 cycle of therapy), to make predictions of response observed at the conclusion of the first regimen of NAT.

The results indicate that various embodiments of the model are superior at predicting tumor response for regimens consisting of only chemotherapy. For the chemotherapy only subgroup, the mathematical model's predictions had greater correlation with the measured tumor response of each patient compared to the chemo+ subgroup, and the model had significantly different predictions between responder and nonresponder patients for all 3 tumor measures. In some embodiments, the model does not explicitly consider the effect of targeted and/or immunotherapies but only implicitly through the calibration of parameters (such as the proliferation map). Therefore, for these embodiments, the chemo subgroup was selected for the in silico alternative regimen investigation. In various embodiments, modeling efforts could account for the effects of targeted therapies to enhance the generalizability of the methodology to all breast cancer subtypes.

Using the parameters derived from each individual patient of the chemo subgroup, several alternative treatment regimens were assessed where the frequency and dosage of drug differed (but the total amount of drug remained consistent) from the standard-of-care regimen each patient received. Therefore, the mathematical model served as an in silico twin for each patient, and an in silico clinical trial was performed with 4 branches—each branch testing an alternative schedule for the total standard-of-care dose and time course. The overall tumor control achieved by the group of standard regimens was found to be statistically inferior to the group of optimal regimens selected for each patient. Because no single regimen may be optimal for all patients, success of any particular treatment approach depends on tumor characteristics of the individual patient and the importance of identifying patient-specific treatment protocols to maximize or otherwise enhance response. Various embodiments of the disclosed patient-specific mathematical modeling systematically exploit this realization for individually optimizing response. Furthermore, it is noted that other possible alternative regimens could be employed to identify patient-specific therapeutic regimens that maximize efficacy.

An aim of this study was to demonstrate use of biophysical, mathematical methods for optimizing therapies on a patient specific basis. The above analyses presented indicated that the mathematical model's predictions are both significantly correlated with and predictive of actual response. It is also important to note that the results of this study would be extraordinarily difficult to achieve using the methods of artificial intelligence, which typically require vast training databases to identify patterns that emerge at the population (rather than the individual) level.

In various embodiments, the number of data points (i.e., scan times) employed in the model calibrations can be increased to enhance the predictive ability of the approach. In particular, if the tumor changes between the calibration scans are minimal compared to the overall change observed at the conclusion of therapy, the model's ability to capture the overall dynamics may be compromised. Additional data acquired prior to and throughout therapy would enable the model calibration scheme to more accurately determine patient-specific parameter values.

Various embodiments employ a biology-based, mathematical model in predicting tumor response using data obtained from the earliest time points—and from the individual patient—during neoadjuvant regimens. The in silico results illustrate how therapeutic regimens can be tailored, and even optimized, for each patient using a mathematical model and simulation studies. These results personalize patient regimens through quantitative imaging and mathematical modeling.

### Supplemental Material

MRI data acquisition: The two imaging facilities are an outpatient imaging facility and a regional hospital that routinely perform breast MRI but have different service contracts and quality control guidelines. Note again that the repeatability and reproducibility of quantitative MRI at these centers was previously established. Breast MRI employed Siemens 3T scanners (Erlangen, Germany) equipped with an 8- or 16-channel receive double-breast coil (Sentinelle, Invivo, Gainesville, Florida). All images were acquired in the sagittal plane. Diffusion-weighted MRI (DW-MRI) was acquired using a monopolar, single-shot spin echo, echo planar imaging sequence in a diagonal diffusion-encoding direction. Six acquisitions were averaged for b-values of 0 and 200 s/mm², while 18 acquisitions were averaged for a b-value of 800 s/mm². (This allowed for approximately equal signal-to-noise ratios at all b-values.) DW-MRI was acquired over 10 slices with 5 mm thickness and no slice gap. Spectrally selective adiabatic inversion recovery (SPAIR) fat suppression was included for a total scan time of 1 minute 39 seconds. Additional acquisition parameters were repetition time/echo time (TR/TE)=3000/52 ms, flip angle 90°, matrix=128×128 (over a 256×256 mm² field-of-view), and a GeneRalized Autocalibrating Partial Parallel Acquisition (GRAPPA) acceleration of 2.

The MRI protocol also included a high-resolution T₁-weighted, 3D gradient-echo, FLASH (fast low angle shot) acquisition using the following parameters: TR/TE=5.3/2.3 milliseconds, flip angle=10°, acquisition matrix=256×256, slice thickness=1 mm, GRAPPA acceleration of 2, and SPAIR (Spectral Selection Attenuated Inversion Recovery) fat suppression. Acquisition time for the anatomical image was 3 minutes and 11 seconds. The DCE-MRI protocol consisted of a T₁-weighted, VIBE (Volumetric Interpolated Breath-hold Examination; though no breath-holding was employed in these studies) acquisition with TR/TE=7.02/4.6 ms, a flip angle of 6°, matrix=192×192, with 10 slices of 5 mm thickness each, and a GRAPPA acceleration factor of 3 yielding a temporal resolution of 7.27 seconds for 1 minute prior to and 6 minutes post administration of a gadolinium-based contrast agent (Multihance (Bracco, Monroe Township, NJ) or Gadovist (Bayer, Leverkusen, Germany)) via a power injector followed by a saline flush. A Siemens TurboFLASH sequence was used to map the B₁ field to correct for transmit inhomogeneity with the following acquisition parameters: TR/TE=8680/2 milliseconds, flip angle=8°, matrix=96×96, and slice thickness=5 mm. Due to the inclusion of a slice gap in the B₁ mapping protocol, two acquisitions were performed to cover the same field-of-view as the above measurements for a total acquisition time of 34 seconds. See Table S.1 for a summary of all imaging parameters.

TABLE S.1
Breast MRI acquisition parameters. All breast MRI data were acquired in the sagittal plane
with a field of view of 256 × 256 mm. For the DW-MRI, six acquisitions were averaged for
b-values of 0 and 200 s/mm2, while 18 acquisitions were averaged for a b-value of 800 s/mm2.
DCE-MRI data had a temporal resolution of 7.27 seconds.

| | single-shot spin | | | |
|---|---|---|---|---|
| | echo, echo | | 3D gradient- | T₁-weighted, |
| Scan sequence | planar | TurboFLASH | echo FLASH | VIBE |
| TR (ms) | 3000 | 8680 | 5.3 | 7.02 |
| TE (ms) | 52 | 2 | 2.3 | 4.6 |
| Flip angle | 90 | 8 | 20 | 6 |
| Acquisition | 128 × 128 | 96 × 96 | 256 × 256 | 192 × 192 |
| Slice thickness | 5 | 5 | 1 | 5 |
| GRAPPA | 2 | N/A | 2 | 3 |
| acceleration | | | | |
| factor | | | | |
| Fat suppression | SPAIR | SPAIR | SPAIR | N/A |

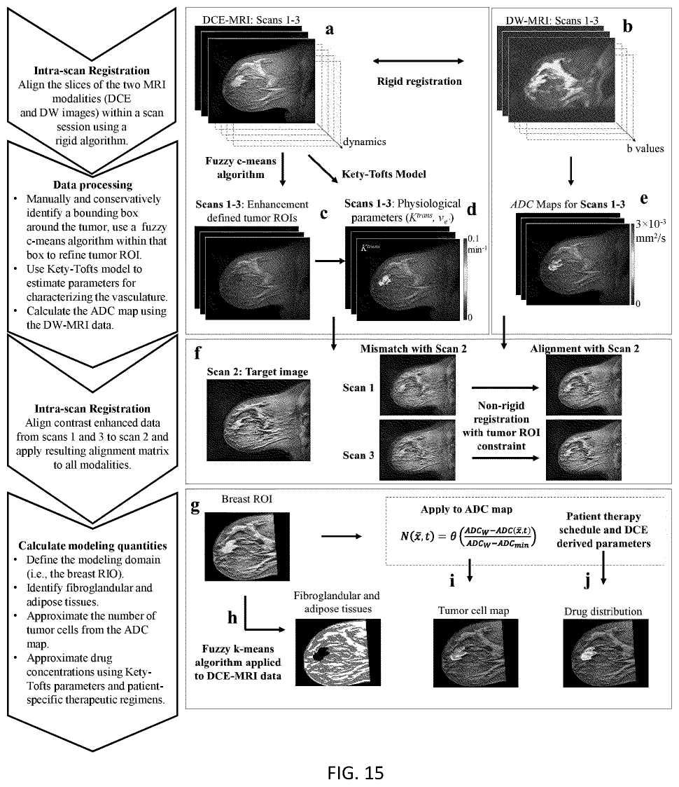

Data analysis techniques: FIG. 15 provides a summary of data analysis and processing steps. Two types of image registrations were performed on each data set: intra-scan (registration within a single visit) and inter-scan (registration across visits). For each patient, the MR images acquired at each session (intra-scan) were aligned using a rigid registration algorithm where the B₁, T₁, and diffusion-weighted images were registered to the DCE images. Images acquired at a different resolution compared to the DCE images were upsampled using a nearest neighbor approach via MATLAB's (MathWorks, Natick, MA) function interp3. The rigid algorithm used for the intra-scan registration was implemented using MATLAB's function imregister. For each patient, all image data sets were registered across time (inter-scan) to a common space via a non-rigid registration algorithm with a constraint that preserves the tumor volumes at each time point. The inter-scan registration employs an adaptive basis algorithm performed using the software Elastix.

The DCE-MRI data were used for both tissue segmentation and characterizing the vasculature for each patient. The DCE-MRI data were used to segment the tumor region of interest (ROI) at each time point using a fuzzy c-means-based clustering algorithm. The clustering algorithm partitions the voxels into classes based on a probability weighting for likely voxel membership to the tumor ROI. To generate masks of fibroglandular and adipose tissues, MATLAB's adapthisteq function was first applied to enhance the post inter-scan registered DCE images, which uses a contrast-limited adaptive histogram equalization algorithm. The fibroglandular and adipose tissues were segmented using a k-means clustering algorithm (these masks are used for the assignment of tissue stiffness properties in the mathematical model detailed below).

The DCE-MRI data were analyzed using the standard Kety-Tofts model:

$${C}_{t} ( \overline{x} , t ) = {K}^{trans} ( \overline{x} ) {\int }_{0}^{t} ( {C}_{p} ( u ) \cdot exp ( - \frac{{K}^{t r a n s} ( \overline{x} )}{{v}_{e} ( \overline{x} )} ( t - u ) ) ) du , \qquad ( S1 )$$

where Cₜ(x̅,t) and Cₚ(t) are the concentrations of the contrast agent in the tissue and plasma at position x̅ and time t, respectively; K^{trans}(x̅) is the volume transfer constant from the plasma space to the tissue space at position x, and vₑ(x̅) is the volume fraction of the extravascular extracellular space at position x. Eq. (S1) was fit to the DCE-MRI data from each voxel within the tumor using a population averaged Cₚₗₐₛₘₐ(t) that was established from the present data set according to the method developed in Li, X., et al., A novel AIF tracking method and comparison of DCE-MRI parameters using individual and population-based AIFs in human breast cancer. Phys Med Biol, 2011. 56(17): p. 5753-69. Voxels for which Eq. (S1) did not converge or converged to non-physical values (i.e., K^{trans}, vₑ<0, or vₑ>1) were removed.

To approximate the drug distribution in each voxel of tissue, a normalized map of the blood volume is calculated by computing the area under the dynamic curve (AUC) of the baseline-subtracted time course for each voxel, and then normalizing by the maximum AUC value from the whole tumor ROI. This normalized blood volume map is then scaled by the peak concentration of drug (as estimated from the Kety-Tofts model) to define the initial drug distribution throughout the domain at the time of each dose of therapy. Specifically, the Kety-Tofts model (i.e., Eq. (S1)) and the DCE-MRI derived physiological parameters per voxel are used where the concentration of contrast agent in the plasma is replaced with the concentration of drug in the plasma from measured population curves for each therapy. Therefore, the concentration of drug in the tumor tissue is spatially non-uniform and temporally varying based on the individual patient's NAT schedule and vascular characteristics.

The apparent diffusion coefficient (ADC) values for individual voxels were calculated using the DW-MRI data via standard methods. Then the ADC value for each voxel within the tumor (as segmented using the above methods) was converted to an estimate for the number of tumor cells per voxel at each 3D position x̅, and time t, N_{TC}(x̅,t), via established methods via Eq. 11, where ADC_{w} is the ADC of free water (3×10⁻³ mm²/s), ADC(x̅,t) is the ADC value for the voxel at position x and time t, and ADCₘᵢₙ is the minimum ADC value over all tumor voxels for the patient. The parameter θ is the carrying capacity describing the maximum number of tumor cells that can physically fit within a voxel; its numerical value is determined by assuming aspherical packing density of 0.7405, a nominal tumor cell radius of 10 μm, and the voxel volume (8.45 mm³).

The tumor volume was approximated as the product of the total number of voxels within the segmented tumor ROI and the DCE-MRI voxel volume. To calculate the longest axis of each tumor, the 3D tumor ROI was evaluated by MATLAB's regionprops3 function, which approximates the longest possible axis within a 3D object. These measures of tumor volume and longest axis are also applied to all the model's predictions (where the model uses the same domain as the MR images) for direct comparison to the experimentally measured values.

Model: A 3D mathematical model that includes the mechanical coupling of tissue properties to tumor growth and the delivery of therapy was designed to be initialized with patient-specific, quantitative, MRI data for breast cancer to predict therapy response. This previous approach is extended to account for multiple chemotherapy terms; see Eq. (12) for the governing equation for the spatiotemporal evolution of tumor, where the first term on the right-hand side describes tumor cell movement, the second term describes the logistic growth of the cells, and the third term describes the effect of chemotherapy. (See Tables S.1 and S.2 for descriptions of the variables and parameters, as well as how they are assigned.) For the growth term, k(x̅) is a spatially resolved proliferation rate map for tumor cells, and the parameter θ is the carrying capacity for logistic growth and is defined as in the previous supplemental section, while the proliferation rate is calibrated per voxel for each individual patient. The first term of Eq. (S3), representing the random diffusion (movement) of the tumor cells, D(x̅,t), is mechanically linked to the breast tissue's material properties via Eq. (2), where D(x̅,t) is the diffusion coefficient, D₀ the tumor cell diffusion constant in the absence of stress (please note that D₀ is the diffusion value for the tumor cells and is not to be confused with the ADC, which is the apparent diffusion coefficient of water molecules), γ an empirical coupling constant for the von Mises stress, σᵥₘ. The von Mises stress reflects the total stress experienced for a given section of tissue; while it is often used as failure criterion in material testing, here it is used to reflect the interaction between the growing tumor and its environment. Therefore, when there is no stress, the diffusion coefficient is equal to D₀. Additionally, a linear elastic, isotropic equilibrium between the tumor and its environment dependent upon changes in tumor cell number is enforced through Eq. (3). Briefly, this model describes tumor growth changes that can cause deformations in the surrounding healthy tissues (breast fibroglandular tissue and adipose tissue), potentially increasing stress and therefore reducing the outward expansion of the tumor.

The therapy term in Eq. (12) describes the spatiotemporal distribution of each drug in the tissue and its effect on the cells of each voxel. Here the model is expanded to acknowledge their differing efficacies and decay rates using Eq. (13). All simulation codes and numerical calculations were written and executed in MATLAB (MathWorks, Natick, MA). The model was implemented in three dimensions (3D) using a fully explicit finite difference scheme with Δt=0.25 day with the mesh dimensions defined by the size of the DCE-MRI voxels. The size of the computational domain is set by a rectangle whose dimensions are determined by the size of the breast for each patient. To reduce computation time for all calibrations, the voxel matrix within this designated rectangular domain was down sampled by a factor of two. A no flux boundary condition was prescribed at the boundary of the breast. To calibrate model parameters, a Levenberg-Marquardt (LM) least squares non-linear optimization is used, where the sum of squared errors between the simulated tumor cell numbers from the model and the calculated tumor cell densities from the imaging data is minimized.

TABLE S.2
Description of the variables of the model system including the
assigned parameter values and specification of units.

| Variable | Description | How Assigned |
|---|---|---|
| N_{TC}(x̅, t) | Number of tumor cells in the voxel at | Derived from the |
| | position x̅ at time t | DW-MRI data |
| D(x,t) | Diffusion coefficient of tumor cells, where | Coupled to the breast |
| | D = D₀exp(−γσᵥₘ(x̅, t)) (mm²/day) | tissues |
| σ^{νm} | Von Mises stress (kPa) | Calculated |
| [symbol] (x̅, t) | Displacement vector due to tumor | Calculated |
| | cell growth (mm) | |
| G | Shear modulus due to breast tissue | Calculated |
| | properties, where G = E/(2(1 − v)) (kPa) | |
| Cₜᵢₛₛᵤₑ^{drug} | Concentration of drug in the | Derived from the |
| (x̅, t) | tissue in the voxel at | DCE-MRI data |
| | position x̅ at time t (μM) | |

TABLE S.3
Description of the parameters for the model system including the
assigned values and specification of units.

| Parameter | Description | How Assigned |
|---|---|---|
| D₀ | Diffusion coefficient of | Calibrated, mm²/day |
| | tumor cells without stress | |
| γ | Mechanical coupling | Assigned at 2.0 × 10⁻³ |
| | coefficient for stress | (1/kPa) |
| v | Poisson's ratio | Assigned, 0.45 |
| | | (dimensionless) |
| E | Young's modulus for adipose, | Assigned, 4 kPa, 2kPa, |
| | fibroglandular, and tumor tissues | and 20 kPa, |
| | | respectively |
| λ | Coupling constant for | Assigned as 2.5 × 10⁻³ |
| | displacement oftumor cells | (dimensionless) |
| k(x̅) | Proliferation rate of tumor | Calibrated, 1/day |
| | cells per voxel | |
| θ | Carrying capacity of tumor cells | Calculated, |
| | in the voxel at position x̋ | 2.02 × 10⁶ cells |
| αβ | Efficacy of the drug against | Calibrated, |
| | tumor cells | 1/(μM · day) |
| | Drug exponential decay rate | Calibrated, 1/day |

Statistical analysis: To test the accuracy of the model's predictions, we generate a Monte Carlo estimated p value for testing the significance of the average absolute difference between the model's predictions and measured outcomes versus random sampling. This is a standard bootstrap random resampling approach. For this particular application, we test the null hypothesis that the mean difference is not equal to a delta value of 10%, 15%, or 20% error (determined using the established variation in the MRI measurements) via the following steps:

(1) Calculate the absolute difference between the model's prediction and measured tumor response for each patient (N=18). (2) Calculate the average absolute difference for the cohort. (3) Sample 500 times with replacement (sample size N=18) the absolute differences of step 1. (4) Calculate the average of each of the 500 samples constructed in step 3. (5) Subtract the corresponding average delta from the average of each of the 500 samples. (6) Calculate the proportion of times each delta adjusted sample average is less than the cohort average calculated in step 2. (7) Subtract the proportion calculated in step 6 from 1.0 to determine the Monte Carlo estimated p value.

These p values are calculated for each of the tumor response measures (i.e., total cellularity, volume, and longest axis).

TABLE S.4
Measured percent changes in total tumor cellularity, volume, and longest axis
betweenscans. Highlighted patients were designated as “responders” by the RECIST
criteria (CR or PR) applied to changes in longest dimension between scan 1 and scan 3.

| | Tumor cellularity | | Tumor volume | | Longest axis percent | |
|---|---|---|---|---|---|---|
| | percent change | | percent change | | change | |
| | Scan 1 to | Scan 1 to | Scan 1 to | Scan 1 to | Scan 1 to | Scan 1 to |
| Patient | Scan 2 | Scan 3 | Scan 2 | Scan 3 | Scan 2 | Scan 3 |
| 1 | — | — | — | — | — | — |
| 2 | −3 | −75 | −2 | −74 | −2 | −5 |
| 3 | −31 | −17 | −25 | −12 | −8 | −10 |
| [symbol] | −33 | −100 | −37 | −100 | −14 | −100 |
| 5 | −15 | −13 | 0 | −18 | 23 | 7 |
| 6 | 11 | −6 | −1 | −23 | −14 | −11 |
| 7 | — | — | — | — | — | — |
| [symbol] | −31 | −81 | −32 | −87 | −12 | −41 |
| 9 | −52 | −69 | −47 | −60 | −16 | −18 |
| [symbol] | −27 | −67 | −21 | −70 | −25 | −43 |
| | −66 | −84 | −65 | −81 | −22 | −52 |
| 12 | −73 | −37 | −74 | −59 | −34 | 32 |
| 13 | 44 | 21 | −20 | −24 | −7 | −13 |
| [symbol] | −73 | −86 | −75 | −86 | −65 | −63 |
| 15 | −14 | −62 | −21 | −63 | −4 | −22 |
| 16 | −8 | — | −5 | — | −3 | — |
| [symbol] | −43 | −30 | −47 | −39 | −44 | −37 |
| | −39 | −53 | −40 | −60 | −24 | −37 |
| 19 | −24 | −64 | −21 | −67 | −10 | −23 |
| [symbol] | −4 | −79 | 1 | −78 | 23 | −33 |
| 21 | −52 | −89 | −40 | −89 | −12 | −25 |
| Median | −27 | −65 | −25 | −65 | −12 | −24 |
| Quartile Range | [−52, −4] | [−82, −26] | [−47, −5] | [−82, −36] | [−24, −4] | [−41, −11] |

Various operations described herein can be implemented on computer systems having various design features. FIG. 16 shows a simplified block diagram of a representative server system 1600 (e.g., computing devices that analyze MRI data) and client computer system 1614 (e.g., imaging devices and sensors, and/or computing devices that receive and present analysis results) usable to implement certain embodiments of the present disclosure. In various embodiments, server system 1600 or similar systems can implement services or servers described herein or portions thereof. Client computer system 1614 or similar systems can implement clients described herein.

Server system 1600 can have a modular design that incorporates a number of modules 1602 (e.g., blades in a blade server embodiment); while two modules 1602 are shown, any number can be provided. Each module 1602 can include processing unit(s) 1604 and local storage 1606.

Processing unit(s) 1604 can include a single processor, which can have one or more cores, or multiple processors. In some embodiments, processing unit(s) 1604 can include a general-purpose primary processor as well as one or more special-purpose co-processors such as graphics processors, digital signal processors, or the like. In some embodiments, some or all processing units 1604 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 1604 can execute instructions stored in local storage 1606. Any type of processors in any combination can be included in processing unit(s) 1604.

Local storage 1606 can include volatile storage media (e.g., conventional DRAM, SRAM, SDRAM, or the like) and/or non-volatile storage media (e.g., magnetic or optical disk, flash memory, or the like). Storage media incorporated in local storage 1606 can be fixed, removable or upgradeable as desired. Local storage 1606 can be physically or logically divided into various subunits such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 1604 need at runtime. The ROM can store static data and instructions that are needed by processing unit(s) 1604. The permanent storage device can be a non-volatile read-and-write memory device that can store instructions and data even when module 1602 is powered down. The term “storage medium” as used herein includes any medium in which data can be stored indefinitely (subject to overwriting, electrical disturbance, power loss, or the like) and does not include carrier waves and transitory electronic signals propagating wirelessly or over wired connections.

In some embodiments, local storage 1606 can store one or more software programs to be executed by processing unit(s) 1604, such as an operating system and/or programs implementing various server functions or computing functions, such as any functions of any components of FIGS. 1 and 12 or any other computing device, computing system, and/or sensor identified in this disclosure.

“Software” refers generally to sequences of instructions that, when executed by processing unit(s) 1604 cause server system 1600 (or portions thereof) to perform various operations, thus defining one or more specific machine embodiments that execute and perform the operations of the software programs. The instructions can be stored as firmware residing in read-only memory and/or program code stored in non-volatile storage media that can be read into volatile working memory for execution by processing unit(s) 1604. Software can be implemented as a single program or a collection of separate programs or program modules that interact as desired. From local storage 1606 (or non-local storage described below), processing unit(s) 1604 can retrieve program instructions to execute and data to process in order to execute various operations described above.

In some server systems 1600, multiple modules 1602 can be interconnected via a bus or other interconnect 1608, forming a local area network that supports communication between modules 1602 and other components of server system 1600. Interconnect 1608 can be implemented using various technologies including server racks, hubs, routers, etc.

A wide area network (WAN) interface 1610 can provide data communication capability between the local area network (interconnect 1608) and a larger network, such as the Internet. Conventional or other activities technologies can be used, including wired (e.g., Ethernet, IEEE 802.3 standards) and/or wireless technologies (e.g., Wi-Fi, IEEE 802.11 standards).

In some embodiments, local storage 1606 is intended to provide working memory for processing unit(s) 1604, providing fast access to programs and/or data to be processed while reducing traffic on interconnect 1608. Storage for larger quantities of data can be provided on the local area network by one or more mass storage subsystems 1612 that can be connected to interconnect 1608. Mass storage subsystem 1612 can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage subsystem 1612. In some embodiments, additional data storage resources may be accessible via WAN interface 1610 (potentially with increased latency).

Server system 1600 can operate in response to requests received via WAN interface 1610. For example, one of modules 1602 can implement a supervisory function and assign discrete tasks to other modules 1602 in response to received requests. Conventional work allocation techniques can be used. As requests are processed, results can be returned to the requester via WAN interface 1610. Such operation can generally be automated. Further, in some embodiments, WAN interface 1610 can connect multiple server systems 1600 to each other, providing scalable systems capable of managing high volumes of activity. Conventional or other techniques for managing server systems and server farms (collections of server systems that cooperate) can be used, including dynamic resource allocation and reallocation.

Server system 1600 can interact with various user-owned or user-operated devices via a wide-area network such as the Internet. An example of a user-operated device is shown in FIG. 16 as client computing system 1614. Client computing system 1614 can be implemented, for example, as a consumer device such as a smartphone, other mobile phone, tablet computer, wearable computing device (e.g., smart watch, eyeglasses), desktop computer, laptop computer, and so on.

For example, client computing system 1614 can communicate via WAN interface 1610. Client computing system 1614 can include conventional computer components such as processing unit(s) 1616, storage device 1618, network interface 1620, user input device 1622, and user output device 1624. Client computing system 1614 can be a computing device implemented in a variety of form factors, such as a desktop computer, laptop computer, tablet computer, smartphone, other mobile computing device, wearable computing device, or the like.

Processor 1616 and storage device 1618 can be similar to processing unit(s) 1604 and local storage 1606 described above. Suitable devices can be selected based on the demands to be placed on client computing system 1614; for example, client computing system 1614 can be implemented as a “thin” client with limited processing capability or as a high-powered computing device. Client computing system 1614 can be provisioned with program code executable by processing unit(s) 1616 to enable various interactions with server system 1600 of a message management service such as accessing messages, performing actions on messages, and other interactions described above. Some client computing systems 1614 can also interact with a messaging service independently of the message management service.

Network interface 1620 can provide a connection to a wide area network (e.g., the Internet) to which WAN interface 1610 of server system 1600 is also connected. In various embodiments, network interface 1620 can include a wired interface (e.g., Ethernet) and/or a wireless interface implementing various RF data communication standards such as Wi-Fi, Bluetooth, or cellular data network standards (e.g., 3G, 4G, LTE, 5G, etc.).

User input device 1622 can include any device (or devices) via which a user can provide signals to client computing system 1614; client computing system 1614 can interpret the signals as indicative of particular user requests or information. In various embodiments, user input device 1622 can include any or all of a keyboard, touch pad, touch screen, mouse or other pointing device, scroll wheel, click wheel, dial, button, switch, keypad, microphone, and so on.

User output device 1624 can include any device via which client computing system 1614 can provide information to a user. For example, user output device 1624 can include a display-to-display images generated by or delivered to client computing system 1614. The display can incorporate various image generation technologies, e.g., a liquid crystal display (LCD), light-emitting diode (LED) including organic light-emitting diodes (OLED), projection system, cathode ray tube (CRT), or the like, together with supporting electronics (e.g., digital-to-analog or analog-to-digital converters, signal processors, or the like). Some embodiments can include a device such as a touchscreen that function as both input and output device. In some embodiments, other user output devices 1624 can be provided in addition to or instead of a display. Examples include indicator lights, speakers, tactile “display” devices, printers, haptic devices (e.g., tactile sensory devices may vibrate at different rates or intensities with varying timing), and so on.

Some embodiments include electronic components, such as microprocessors, storage and memory that store computer program instructions in a computer readable storage medium. Many of the features described in this specification can be implemented as processes that are specified as a set of program instructions encoded on a computer readable storage medium. When these program instructions are executed by one or more processing units, they cause the processing unit(s) to perform various operation indicated in the program instructions. Examples of program instructions or computer code include machine code, such as is produced by a compiler, and files including higher-level code that are executed by a computer, an electronic component, or a microprocessor using an interpreter. Through suitable programming, processing unit(s) 1604 and 1616 can provide various functionality for server system 1600 and client computing system 1614, including any of the functionality described herein as being performed by a server or client, or other functionality associated with message management services.

It will be appreciated that server system 1600 and client computing system 1614 are illustrative and that variations and modifications are possible. Computer systems used in connection with embodiments of the present disclosure can have other capabilities not specifically described here. Further, while server system 1600 and client computing system 1614 are described with reference to particular blocks, it is to be understood that these blocks are defined for convenience of description and are not intended to imply a particular physical arrangement of component parts. For instance, different blocks can be but need not be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus including electronic devices implemented using any combination of circuitry and software.

In various embodiments, the codes may be implemented with the logic of CPU-based programming. Multi-core CPUs may be deemed beneficial if some steps are to be run in parallel, though this is not necessary (nor is multi-node). In some of datasets, all images from one visit may take, for example, approximately 3 gigabytes (GB) of memory, and if there are 3 visits each patient, approximately 10 GB may be allocated to preserve similar performance with respect to computational time for certain embodiments discussed above.

### Additional Examples: Mri-Based Digital Models Forecast Patient-Specific Treatment Responses to Neoadjuvant Chemotherapy in Triple-Negative Breast Cancer

Triple-negative breast cancer (TNBC) is persistently refractory to therapy, and methods to improve targeting and evaluation of responses to therapy in this disease are needed. According to potential embodiments, Applicants integrate quantitative magnetic resonance imaging (MRI) data with biologically-based mathematical modeling to accurately predict the response of TNBC to neoadjuvant systemic therapy (NAST) on an individual basis. Specifically, 56 TNBC patients enrolled in the ARTEMIS trial (NCT02276443) underwent standard-of-care doxorubicin/cyclophosphamide (A/C) and then paclitaxel for NAST, where dynamic contrast-enhanced MRI and diffusion-weighted MRI were acquired before treatment and after two and four cycles of A/C. A biologically-based model was established to characterize tumor cell movement, proliferation, and treatment-induced cell death. Two evaluation frameworks were investigated using: 1) images acquired before and after two cycles of A/C for calibration and predicting tumor status after A/C, and 2) images acquired before, after two cycles, and after four cycles of A/C for calibration and predicting response following NAST. For Framework 1, the concordance correlation coefficients between the predicted and measured patient-specific, post-A/C changes in tumor cellularity and volume were 0.95 and 0.94, respectively. For Framework 2, the biologically-based model achieved an area under the receiver operator characteristic curve of 0.89 (sensitivity/specificity=0.72/0.95) for differentiating pathological complete response (pCR) from non-pCR, which is statistically superior (P<0.05) to the value of 0.78 (sensitivity/specificity=0.72/0.79) achieved by tumor volume measured after four cycles of A/C. Overall, this model successfully captured patient-specific, spatiotemporal dynamics of TNBC response to NAST, providing highly accurate predictions of NAST response.

Integrating MRI data with biologically-based mathematical modeling successfully predicts breast cancer response to chemotherapy, indicating digital twins could facilitate a paradigm shift from simply assessing response to predicting and optimizing therapeutic efficacy.

Introduction: Neoadjuvant systemic therapy (NAST) is widely considered the standard-of-care for treatment of stage II-III, locally advanced triple-negative breast cancer (TNBC). NAST increases the success rate for breast conservation surgery by reducing tumor burden and provides the opportunity to treat micrometastases in a naïve state, thereby improving progression-free survival of patients. Importantly, TNBC patients who achieve a pathological complete response (pCR) in the neoadjuvant setting have a favorable recurrence-free survival; in contrast, patients who have residual disease after NAST are at increased risk of early recurrence and death. Unfortunately, based on recent pooled analysis of 52 studies from 1999 to 2016, only 32.6% of TNBC patients treated with standard taxane/anthracycline-based NAST have a pCR or minimal residual disease at the time of surgical resection.

Development of novel neoadjuvant treatment regimens has provided opportunities to tailor treatment for individual patients to improve outcomes for TNBC. Thus, it is becoming increasingly important to develop techniques which can accurately predict the individual TNBC patient's response to NASTs. If it could be definitively determined that a therapeutic regimen is unlikely to achieve pCR for a patient, then risk-adapted therapy could be adopted, with addition of rationally-based treatments or removal of unnecessary components, potentially improving outcomes and lowering side effects. In particular, the importance of being able to remove patients from ineffective therapies, as early as possible, is difficult to overstate given their significant toxicities, including increased likelihood of hospitalization, cardiac damage, leukemia, and even death. Moreover, accurately predicting response to NAST would enable identification of exceptional responders who might benefit from treatment de-escalation, including the possibility of non-surgical management of their disease.

Numerous efforts have been devoted to investigating approaches that can accurately distinguish pCR from non-pCR patients early in NAST. Imaging biomarkers derived from magnetic resonance imaging (MRI), positron emission tomography, and ultrasound imaging have been shown to be strongly correlated with the response of breast tumors to NAST. In particular, MRI measurements before and during NAST have been valuable predictors of pCR, especially the functional tumor volume (FTV) derived from dynamic contrast-enhanced (DCE-) MRI and the apparent diffusion coefficient (ADC) derived from diffusion-weighted (DW-) MRI. More recently, the methods of artificial intelligence have been used to extract features from high-dimensional data to build predictive models for differentiating pCR from non-pCR in breast cancer. However, the majority of these approaches for predicting or assessing response have the intrinsic limitation of being population-based. Importantly, population-based approaches, which rely exclusively on statistical inference from properties of large populations, inevitably obscure conditions specific to the individual patient over time, especially for a disease as heterogeneous as cancer. Conversely, biologically-based models employing patient-specific data have the potential to shift the paradigm from population- to individual-based approaches. Furthermore, biologically-based mathematical modeling of tumor response can not only predict the changes in global metrics summarizing tumor burden (e.g., total tumor volume and cellularity), but also reveal biologically specific information (e.g., spatially-resolved maps of proliferation, pharmacokinetics, and each patient's sensitivity to the administered therapies). It promises unique opportunities to characterize tumor pathophysiology, to rigorously forecast long-term outcomes, and even optimize treatment plans on a patient-specific basis.

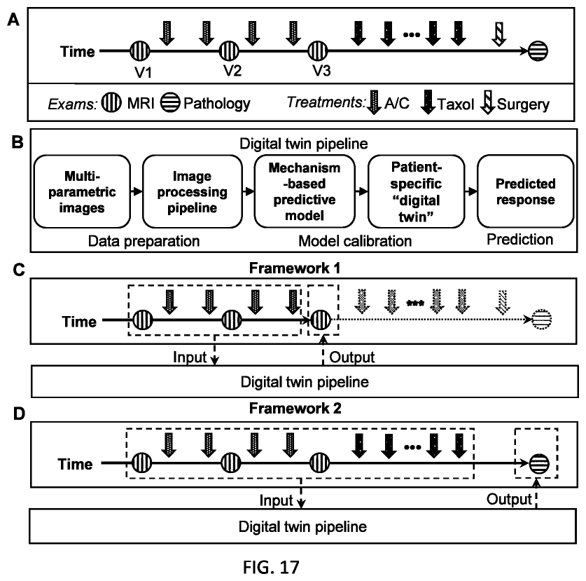

As discussed herein, according to various potential embodiments, Applicants have develop a clinical-computational approach to establish patient-specific models to make early predictions on the spatiotemporal development and response of individual TNBC patients to standard-of-care NAST. As this model can represent a physical object (i.e., tumor), predict the behavior of the object given influences (i.e., treatments), and enable decision-making to optimize the future behavior of the object (i.e., improve the treatment outcomes), we posit that our methodology represents a practical manifestation of digital twins in oncology. The approach requires no population-based training of models; instead, it integrates an individual patient's multiparametric MRI data obtained at multiple time points during their treatment (FIG. 17A) with biologically-based mathematical modeling. In particular, two frameworks were constructed to determine the predictive utility of each patient's digital twin (FIG. 17B). In Framework 1, we seek to employ digital twins to predict the outcome of a single NAST regimen (i.e., A/C); specifically, we evaluate the accuracy of digital twins to predict global metrics related to change in tumor burden, as well as spatiotemporally resolved tumor dynamics at the end of A/C (FIG. 17C). In Framework 2, we seek to employ digital twins to predict the outcome of the entire NAST (i.e., both A/C and paclitaxel); specifically, we evaluate the accuracy of digital twins to predict individual pCR or non-pCR status at the completion of NAST (FIG. 17D).

Patients and MRI Data: Treatment-naïve patients with biopsy-confirmed TNBC were enrolled in the prospective, Institutional Review Board-approved clinical trial, “A Robust TNBC Evaluation FraMework to Improve Survival” (ARTEMIS, NCT02276442). Among patients who signed informed consent between June 2018 to January 2020, those who had clinical stage I-III disease, had completed NAST with a complete series of MRI scans, and had known post-surgical pathology were included in this study (n=56).

Each patient underwent multiparametric MRI acquisitions before treatment (baseline/V1), after two cycles (V2), and after four cycles (V3) of the standard-of-care (neoadjuvant) doxorubicin and cyclophosphamide (A/C). (Each “cycle” of A/C is 2 weeks; see FIG. 17A.) Patients with disease progression, or <70% reduction in tumor volume at the end of A/C, were offered the opportunity to enroll in a biomarker-guided clinical trial using targeted bio/chemotherapy to complete therapy (n=9). Patients not meeting the criteria for suboptimal response to A/C were recommended to continue standard-of-care paclitaxel weekly for 12 cycles (n=43), or double-dosed every 3 weeks for four cycles (n=4). (The exact paclitaxel regimen for each patient's was determined by their physician.) All patients received surgery after NAST. The post-surgical pathology was used to classify patients as pCR or non-pCR; pCR was defined as the absence of residual invasive and in situ cancer on hematoxylin and eosin evaluation of the complete resected breast specimen and all sampled regional lymph nodes following completion of NAST.

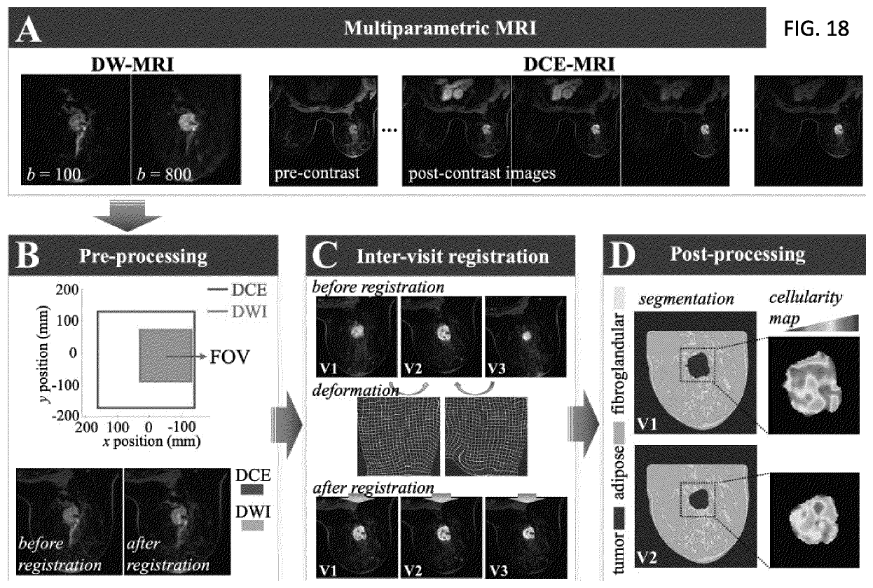

MRI was performed on a GE Discovery MR750 or MR750w whole-body scanner (GE Healthcare) with an eight-channel bilateral breast coil. In particular, the DCE-MRI data was acquired using a 3D DISCO sequence with bipolar readouts (FIG. 18A) and the following scan parameters: field-of-view=30×30 cm², matrix size=320×320, slice thickness/spacing=3.2/−1.6 mm, number of slices=140, flip angle=12°, repetition/echo time=8/2 ms. After one pre-contrast phase was obtained, a single bolus of contrast agent (Gadovist, Bayer HealthCare) was injected (0.1 mL/kg at ˜2 mL/second followed by saline flush) at the start of the post-contrast acquisition. The temporal resolution of the DISCO series ranged from 8 to 15.5 s (median=11 s), depending on the slice coverage, which resulted in the number of post-contrast phases varying from 32 to 64. The DW-MRI was acquired using a 2D spin-echo sequence of the diseased breast with the following scan parameters: field-of-view=16×16 cm², matrix size=80×80, slice thickness/spacing=4/0 mm, number of slices=16, flip angle=90°, repetition/echo time=4000/70 ms. The b-values used were 100 and 800 s/mm². The apparent diffusion coefficient (ADC) map was calculated using a GE AW server (v3.2, GE Healthcare, Milwaukee, WI). The tumors were manually segmented by two board-certified breast radiologists with 4-12 years of experience (authors MB, RMM). Tumor segmentations were reviewed by two breast fellowship trained radiologists with 19 and 20 years of experience (authors GMR, BEA). The entire tumor volume was segmented on two early phases of the DCE-MRI using an in-home software package developed in MATLAB (R2021b, Mathworks, Natick, MA). All segmentations were further refined using the thresholding tool of the package to exclude non-tumor voxels that were determined by radiologists. Necrotic regions and artifacts from the biopsy clip were manually segmented and excluded by two radiologists (authors MB, RMM).

Image processing: According to various potential embodiments, all MRI data from each patient were processed through a pipeline that consists of three components: 1) pre-processing, 2) inter-visit registration, and 3) post-processing (FIGS. 18B-D). This highly automated pipeline allows efficient processing of multi-visit, multi-parametric MRI with minimal user input.

First, the multiparametric images are co-localized to the same imaging grid to align slices and voxel locations. Specifically, DCE-MRI collected bilaterally were trimmed to the DW-MRI field-of-view covering the diseased breast, and the slices of DW-MRI and ADC maps were linearly interpolated to match the slice locations of DCE-MRI. A rigid registration was applied on the DCE-MRI to align all phases in one scan to the pre-contrast phase (MATLAB function, imregtform). A rigid registration was applied between the co-localized DCE-MRI and DW-MRI data to remove the small mismatches between the image volumes.

Second, an inter-visit image registration was performed to account for the change of breast tissue shape and patient position across MRI visits. Specifically, the registration was performed to align the images from V1 and V3 to the images from V2. The algorithm consisted of a rigid registration of the tumor ROIs for initial alignment, followed by a deformable b-spline, non-rigid registration on the whole breast with a rigidity penalty on the tumor regions. This rigidity penalty was imposed to preserve the tumor volume and shape across all visits. This registration was developed based on MATLAB and an open-source, command-line software, Elastix.

Third, the post-processing was performed as preparation for the subsequent predictive modeling. Specifically, a semi-automatic segmentation of the breast contour was performed on the pre-contrast frame of DCE-MRI based on a manually chosen intensity threshold, followed by a smoothing of the segmented mask edge (MATLAB function, imgaussfilt). A two-class k-means clustering (MATLAB function, kmeans) was used to segment fibroglandular and adipose tissues in each pre-contrast DCE-MRI. The enhancement of each DCE-MRI was calculated by subtracting the pre-contrast phase from the average of the post-contrast phases. A tumor cellularity map, N(x,t), was estimated based on the measured ADC map of each MRI visit:

$$N ( x , t ) = θ ( \frac{A D {C}_{w} - A D C ( x , t )}{A D {C}_{w} - A D {C}_{min}} ) \qquad ( T1 )$$

where ADC_{w} is the ADC of free water (3×10⁻³ mm²/s), ADC(x,t) is the ADC value for the voxel at position x and time t, and ADCₘᵢₙ is the minimal (positive) ADC value in the tumor for the patient across all visits. The carrying capacity, θ, describes the maximum number of tumor cells that can physically fit within a voxel, which was determined by assuming a spherical packing density of 0.7405 and a nominal tumor cell radius of 10 μm.

Image-guided biologically-based modeling: Applicants have developed a biologically-based mathematical model to represent the spatiotemporally-resolved dynamics of tumor growth and response to NAST. Specifically, a reaction-diffusion partial differential equation is used to describe the evolution of tumor cells, N(x,t), in response to the therapies, as shown in Eq. (T2):

$${{\frac{\partial N ( x , t )}{\partial t} = {∇ \cdot ( D ( x , t ) ∇ N ( x , t ) )}_{︸}^{︷}}^{Tumor cell diffusion}}_{Movement} + {\overbrace{{\underbrace{k ( x ) ( 1 - \frac{N ( x , t )}{θ} ) N ( x , t )}}_{Proliferation} - {\underbrace{N ( x , t ) {\sum }_{i} {λ}_{i} ( x , t )}}_{treatment - induced death}}}^{Reaction} , \qquad ( T2 )$$

where a detailed list of variables, parameters, their definitions and assignments are given in Table T3 below. The first term on the right-hand side of Eq. (T2) describes the movement of tumor cells, as well as the compression of surrounding tissue, by a diffusion process coupled to tissue mechanical properties via Eq. (T3):

$$D ( x , t ) = {D}_{0} {e}^{- γ σ ( x , t )} , \qquad ( T3 )$$

where Do is the tumor cell diffusion coefficient in the absence of external forces. The exponential term reduces tumor cells' mobility due to the surrounding tissue stiffness via the von Mises stress, σ(x,t), and an empirical coupling constant, γ. The von Mises stress was calculated for the fibroglandular and adipose tissues within the breast, with the fibroglandular tissue assigned a greater stiffness than the adipose tissue. Technical details on the mechanical-coupled diffusion can be found in. The proliferation of the tumor cells (second term on right-hand side of Eq. (T2)) is described by logistic growth with a spatially varying proliferating rate, k(x), and a global carrying capacity, θ. The effects of administered therapies (third term on the right-hand side of Eq. (T3) is modeled as the treatment-induced death rates of tumor cells, λi(x,t). The death rates are determined by the concentration of the administered drugs and the exponential decay of their efficacies:

$${λ}_{i} ( x , t ) = {α}_{i} {\sum }_{j = 1}^{Ji} {e}^{- {β}_{i} ( t - {τ}_{i j} )} C ( x , {τ}_{i j} ) , i = 1 , 2 , and 3 , \qquad ( T4 )$$

where αᵢ is the efficacy of the i^{th} administered drug, and i=1, 2, and 3 refers to doxorubicin, cyclophosphamide, and paclitaxel, respectively. As each drug was administered multiple times during the therapeutic regimen, the total effectiveness of drugs at time t was an accumulation of all administered cycles and their decays; that is, the j^{th} administration of the i^{th} drug was conducted at time τ_{i,j} at a total of Jᵢ times. The decay of drug efficacy from each administration is represented by the decay rate of βᵢ. The spatial distribution of i^{th} drugs caused by the j^{th} administration of this therapy, C(x, τ_{i,j}), is determined by the enhancement of DCE-MRI. Specifically, from the last DCE-MRI data collected before the injection at τ_{i,j}, the area under the DCE-MRI time course is calculated at each voxel and normalized by the maximum value within the tumor. The voxel-wise normalized area under the curve represents the map of drug concentration induced by this injection (see Supplemental Section 1.1 for details).

Eqs. (T2-T4) are personalized by the imaging and clinical data of each patient. Specifically, the geometry of the computational domain is determined by the segmented breast contour, tumor, and fibroglandular/adipose tissues. The tumor cellularity map at each imaging time point is determined from the ADC map obtained at the corresponding time point via Eq. (T1). The sequential cellularity maps are then used for patient-specific calibration of the model parameters (i.e., k(x), D₀, αᵢ, and βᵢ). Additionally, the DCE-MRI acquired during NAST were used for updating the spatial distribution of the administered drugs and the mechanical properties of breast tissues. The model constrained by patient-specific data was implemented in MATLAB and solved with the finite difference method. Certain details of the numerical implementation can be found in Jarrett A M, Kazerouni A S, Wu C, Virostko J, Sorace A G, DiCarlo J C, et al. Quantitative magnetic resonance imaging and tumor forecasting of breast cancer patients in the community setting. Nat Protoc 2021;16(11):5309-5338 and Jarrett A M, Hormuth II D A, Wu C, Kazerouni A S, Ekrut D A, Virostko J, et al. Evaluating patient-specific neoadjuvant regimens for breast cancer via a mathematical model constrained by quantitative magnetic resonance imaging data. Neoplasia 2020;22(12):820-830.

Digital twin frameworks: As shown in FIG. 17C, Framework 1 focuses on employing digital twins to predict the outcome of a single NAST regimen (i.e., A/C). Specifically, for each patient, the processed images from V1 and V2 are imported with the treatment regimen and calibrated to Eqs. (T2-T4). The calibrated model is the digital twin as it represents patient-specific pathophysiological properties of tumor growth and response, including pre-treatment tumor shape and cellularity, the proliferation rate and mobility of tumor cells, and the efficacy and decay of administered drugs (A/C). As the efficacy and decay rates were strongly coupled and challenging to simultaneously calibrate with only two-time-points, the decay rates of A/C were randomly sampled 5 times from ranges found in the published literature: β₁∈[0.01, 0.6] day⁻¹,) β₂∈[1.0, 5.4] day⁻¹. With each set of sampled β₁ and β₂₁, the efficacy of A/C was calibrated, resulting in five parameter sets (see Supplemental Section 2.1). By applying the remainder of the A/C (i.e., all A/C after V2; FIG. 17C) to the digital twin, the patient-specific spatiotemporally-resolved tumor cell distributions are predicted up to the end of A/C; one prediction is given by each parameter set, resulting in a median and a range of predictions. Furthermore, for each patient, global metrics—total tumor cellularity (TTC) and total tumor volume (TTV)—are derived from the spatiotemporally-resolved predictions.

The predictive accuracy of Framework 1 is evaluated both temporally and spatially. The temporal accuracy is assessed by the agreement between the predicted and measured global metrics (i.e., TTC and TTV). In particular, the concordance correlation coefficient (CCC; see Supplemental Section 1.2) is computed between the predicted and measured changes of TTC at the end of A/C; similarly, the CCC is computed between the predicted and measured changes of TTV. The spatial accuracy is assessed by the difference between the predicted and measured spatially-resolved tumor cell distributions. In particular, for each patient, the percent change of tumor cell counts from baseline (V1) to the end of A/C (V3) can be calculated at each location, x. We calculate the change from predicted and measured tumor cell distributions (i.e., ΔTCDₚ(x) and ΔTCDₘ(x), respectively), then the spatially-resolved difference is given by ΔTCDₚ(x)−ΔTCDₘ(x). In each patient, this difference is reported as the median and interquartile range within the original tumor region; across the cohort, the differences are evaluated by the mean and 95% confident interval (CI) of the median difference from each patient. (Considering 5 predictions are given for each patient, these evaluations are based on the median of the 5 predictions.)

As shown in FIG. 17D, Framework 2 focuses on employing digital twins to predict the outcome of the entire NAST (i.e., both A/C and paclitaxel). Specifically, for each patient, the processed images from V1, V2, and V3 are imported into the mechanism-based model for initialization and calibration. The calibrated model is the digital twin. (Note that the three-time-point data enables calibration of the efficacy and decay rates simultaneously within the same ranges assumed in Framework 1, so the sampling scheme in Framework 1 is not needed for Framework 2.) As no imaging data were available during the paclitaxel regimen, the efficacy of paclitaxel was set to literature value, α₃=0.3 day⁻¹, and the decay rate of paclitaxel was assumed as the average of calibrated A/C decay rate;) β₃=(β₁+β₂)/2. (See Supplemental Section 2.1 for details.) By applying paclitaxel to the digital twin, it predicts the patient-specific spatiotemporally-resolved tumor cell distributions, TTC, and TTV at the end of NAST.

The output of Framework 2 is evaluated by the ability of the digital twins to differentiate pCR and non-pCR. Specifically, receiver operating characteristic (ROC) analysis is performed on the predicted TTC and TTV. We report the area under ROC curve (AUC), sensitivity (i.e., the ability to correctly identify residual tumor at final surgical pathology), and specificity (i.e., the ability to correctly identify pCR at final surgical pathology) based on the optimal cut-off. Additionally, the AUC/sensitivity/specificity from the predicted TTC and TTV were compared to those obtained by the measured TTC and TTV. The 95% CIs of the AUCs were computed and compared via DeLong's method, with P<0.05 considered statistically significant.

Results, Framework 1: Patient-specific predictions of the spatiotemporal response to A/C: A cohort of 50 patients was used in Framework 1. Six patients were excluded from the entire patient cohort (n=56) due to image acquisition error or artifacts (n=1), unsuccessful inter-visit registration caused by a large change of breast shape between visits (n=1), and a complete response of the tumor at V2 which led to no data for model calibration (n=4).

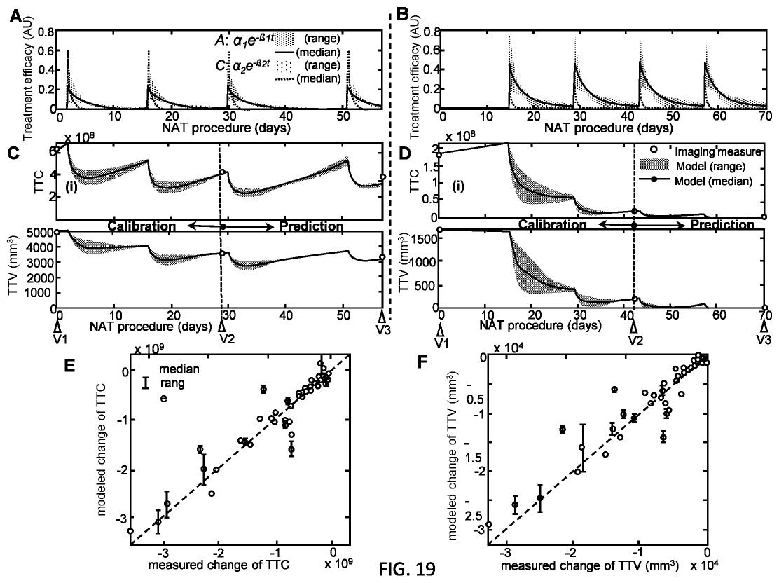

Each patient's digital twin provided a range of estimated treatment efficacies of A/C (e.g., FIG. 19A-B), simulating a range of treatment outcomes (e.g., FIG. 19C-D). Specifically, FIG. 19C presents a patient who had a suboptimal response to A/C (i.e., V3 imaging showed a <70% reduction in tumor volume). The digital twin predicted a mean (range) TTC and TTV of 3.17×10⁸ (3.05×10⁸-3.29×10⁸) cells and 3.19×10³ (3.15×10³-3.23×10³) mm³ at V3, respectively. This corresponds to predicted decreases of 49.55% (47.60%-51.50%) and 36.14% (35.44%-36.84%) for TTC and TTV at V3, respectively. In comparison, the measured decreases are 38.99% and 32.58% for TTC and TTV at V3, respectively. In contrast, FIG. 19D presents a patient who had a good response to A/C (i.e., V3 imaging showed >70% reduction in tumor volume). The digital twin predicted a TTC and TTV of 3.82×10⁶ (1.69×10⁶-5.94×10⁶) cells and 12.63 (0.00-25.27) mm³ at V3, respectively. This corresponds to predicted decreases of 98.00% (96.88%-99.11%) and 99.24% (98.48%-100.00%) for TTC and TTV at V3, respectively. In comparison, the measured decrease is 100% for both TTC and TTV at V3. Across the cohort, the CCC between the predicted and measured changes in TTC at V3 was 0.95 (FIG. 19E); the CCC between predicted and measured changes in TTV was 0.94 (FIG. 19F). These results indicate a high predictive accuracy and precision (i.e., uncertainty in the model's prediction; see Supplemental Section 2.2 for interpretation) of the temporal dynamics of the response of TNBC to neoadjuvant A/C (Table T1).

TABLE T1
Statistical evaluation of patient-specific predictions
of the TNBC response to A/C

| | metric | | |
|---|---|---|---|
| | temporal accuracy | | spatial accuracy |
| | CCC between | CCC between | Mean (95% CI) of the |
| | predicted and | predicted and | median differences |
| time | measured | measured | between predicted and |
| point | ΔTTC^{a} | ΔTTV^{b} | measured ΔTCD^{c} |
| V2 | 1.00 | 0.99 | 0.22% |
| | | | (−3.34%-3.78%) |
| V3 | 0.95 | 0.94 | 0.20% |
| | | | (−20.35%-20.75%) |

Note:
^{a}ΔTTC = change of total tumor cellularity from pre-treatment to time point V2 or V3
^{b}ΔTTV = change of total tumor volume from pre-treatment to time point V2 or V3
^{c}ΔTCD = change of spatially-resolved tumor cell distribution within original tumor region from pre-treatment to time point V2 or V3

Importantly, the personalized digital twins provide not only the global metrics summarized in the previous paragraph, but also the spatiotemporal evolvement of each patient's tumor. FIGS. 20A and 20B show the measured and predicted tumor cell distributions, respectively, from the central slice of the same two example patients; and FIGS. 20C and 20D present the 3D rendering of measured and predicted tumor volumes, respectively. In both cases, the digital twins successfully capture the lack (FIG. 20A) or presence (FIG. 20B) of response. Quantitatively, for the first patient, the difference between the predicted and measured change of tumor cell distributions at V3 has a median (interquartile range) of −3.30% (−22.07%-0.00%); for the second patient, the difference has a median (interquartile range) of 0.00% (0.00%-0.00%). The median differences across all patients had a mean (95% CI) of 0.20% (−20.35%-20.75%) at V3 (FIG. 20E). These results indicate high predictive accuracy and precision of spatially-resolved predictions of tumor cell distributions in TNBC patients in response to neoadjuvant A/C (Table T1).

Results, Framework 2: Patient-specific prediction of final pathological response: A cohort of 37 patients (18 pCR, 19 non-pCR) was used in Framework 2. After excluding the six patients from the entire cohort (n=56) as conducted in Framework 1, thirteen more patients were excluded due to presumed non-response to further standard-of-care chemotherapy and enrollment into clinical trials (n=9; part of ARTEMIS schema), missing the schedule of paclitaxel (n=2), and errors in image acquisition (n=2; ADC maps did not cover the entire tumor at V3).

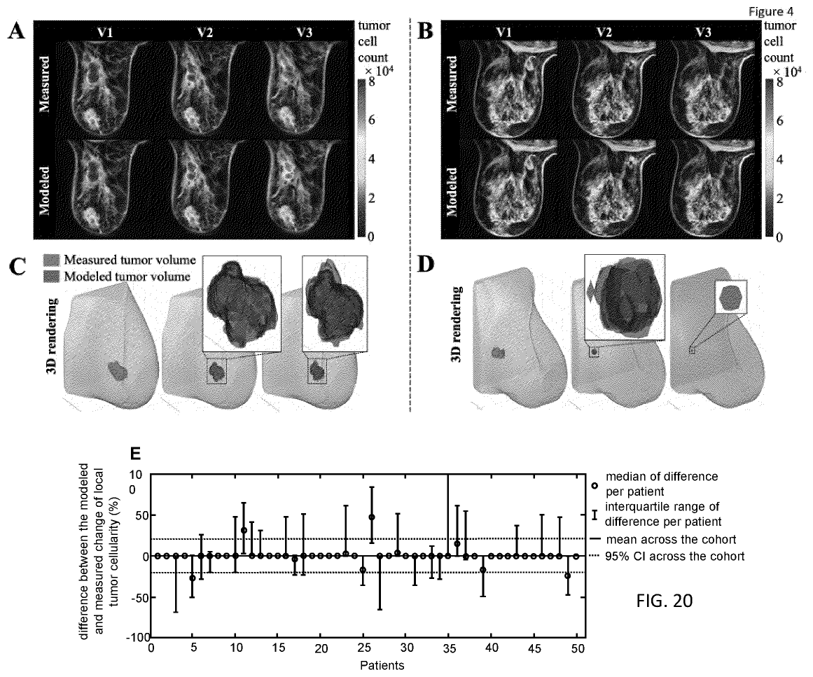

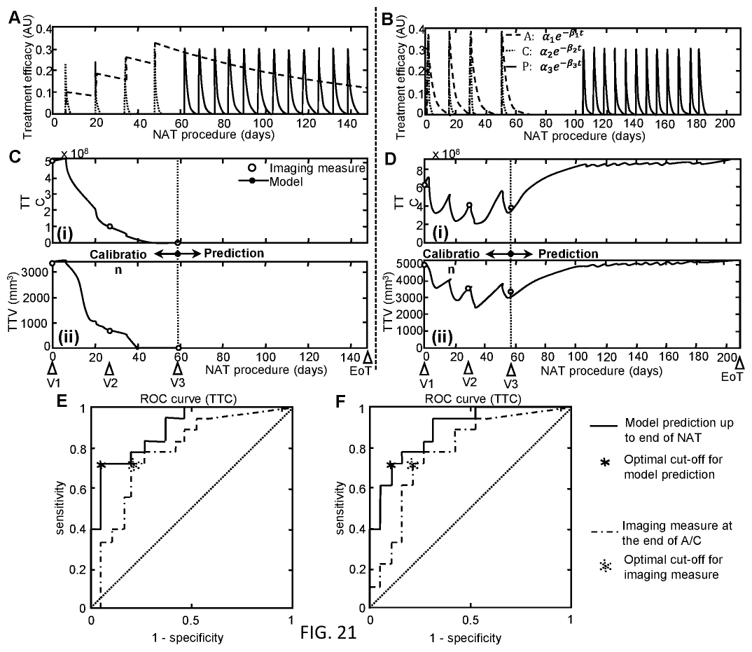

For each patient, the personalized digital twin estimated treatment efficacies based on the V1-V3 images (FIG. 21A), and represented tumor dynamics in response to A/C. These results were then used to predict the response to paclitaxel and, therefore, final treatment outcome after all NAST (FIG. 21C). For example, FIG. 21C shows the digital twin was able to be calibrated during the A/C regimen and used to predict no regrowth during paclitaxel for a patient that did, in fact, achieve pCR after NAST. In contrast, FIG. 21D shows the digital twin captured an initial response and then subsequent regrowth during A/C, and predicted strong regrowth before and during paclitaxel, resulting in a predicted TTC and TTV values of 9.03×10⁸ and TTV of 5.21×10³ mm³, respectively, after NAST.

Across the cohort, as shown in Table T2, the predicted TTC and TTV (based on the model calibrated with the V1-V3 data) from the digital twins both achieved an AUC of 0.89 (0.78-0.99) for distinguishing pCR from non-pCR (with post-surgical pathology as the ground truth). In comparison, the measured TTV or TTC (based on the V3 data) achieved an AUC of 0.78 (0.62-0.94) for distinguishing pCR from non-pCR. Furthermore, using the predicted TTC and TTV, the specificity was 0.95 and 0.89, respectively. In comparison, if using the measured TTV or TTC, the specificity was only 0.79. These results demonstrate that compared to the directly measured data, the digital twins improved the prediction of final response. Specifically, the AUC was improved by 14.28% for TTC (P=0.04; significant) and 13.83% for TTV (P=0.07). The specificity was improved by 20.25% and 12.66% for TTC and TTV, respectively; the sensitivity was unchanged.

TABLE T2
Statistical evaluation of patient-specific prediction
of final pathological response

| | | Statistic | | |
|---|---|---|---|---|
| Metric | | AUC(95% CI) | Sensitivity | Specificity |
| TTC | Measured | 0.75 (0.62-0.94) | 0.72 | 0.79 |
| | Predicted | 0.89 (0.78-0.99) | 0.72 | 0.95 |
| TTV | Measured | 0.78 (0.62-0.94) | 0.72 | 0.79 |
| | Predicted | 0.89 (0.78-0.99) | 0.72 | 0.89 |

Discussion of various embodiments: Potential embodiments of the disclosure provide a digital twin approach to achieve early, patient-specific, spatiotemporally-resolved predictions of the response of TNBC patients to neoadjuvant doxorubicin, cyclophosphamide, and paclitaxel. This approach was based on a biologically-based mathematical model calibrated with multi-visit, multiparametric MRI acquired for the individual patient. Thus, the methodology represents a significant step away from population-based predictions, and towards individual-based predictions.

Framework 1 shows how the digital twin employs the imaging data from individual patients acquired early in the course of neoadjuvant A/C to make very accurate predictions of tumor status at the conclusion of A/C. The CCC between the predicted and measured values of total tumor burden and total tumor volume were 0.95 and 0.94, respectively. This strongly indicates that early changes during the A/C regimen contain sufficient information to calibrate the digital twin and confidently predict tumor response at the end of A/C. This observation aligns with previous reports demonstrating that metrics from early-treatment MRI are strong predictors of NAST response in breast cancer. This provides strong support that our approach could be used to adjust treatment regimens on a patient-specific basis. For example, our approach could be applied after the first two cycles of A/C to predict if further dosing with A/C should be continued, or if an alternative intervention should be considered.

Framework 2 shows how the digital twin employs the imaging data from individual patients acquired during the A/C portion of NAST to make accurate predictions of their final pathological status (i.e., pCR or non-pCR) at the completion of all NAST, with an AUC=0.89. Importantly, using the tumor volume measured at the end of A/C yielded only an AUC=0.78; thus, the digital twin provided a substantial improvement on the AUC (14%). Comparing to previous MRI-based predictions of breast cancer response to NAST, the digital twin also shows an improved accuracy. Both our previous study and an I-SPY study reported the best discrimination between pCR/non-pCR in TNBC using the optimized mid-treatment FTV, with AUC=0.85. Moreover, the addition of post-NAST ADC to FTV showed an improvement for predicting response in TNBC, increasing AUC from 0.71 to 0.81. Combining a pharmacokinetic parameter (i.e., kₑₚ) with ADC measured post one-cycle NAST yielded an AUC=0.88 in breast cancer. Moreover, the predictive accuracy of the digital twin is comparable to state-of-the-art, machine learning (ML)-based predictions. For example, Ravichandran et al. applied a convolutional neural network (CNN) to predict pCR from pre-treatment DCE-MRI and achieved an AUC of 0.77 in a total of 166 breast cancer patients. A more recent CNN-based study using both pre-and post-treatment DCE-MRI achieved an AUC of 0.91 in a cohort of 42 breast cancer patients.

Importantly, embodiments of the disclosed digital twin approach have several inherent advantages comparing to ML algorithms. First, ML methods rely on access to a large patient population to train the algorithm and this training dataset must include all relevant pathophysiological features of the disease under investigation, have high-quality annotations, and be generalizable from one population to the next. In contrast, our approach calibrates a biologically-based model using patient-specific data to make patient-specific predictions, which does not require population-based training or annotation labels. Second, ML can be difficult to interpret biologically due to complex modeling features. In contrast, the digital twins provide an accurate prediction not only of pCR status at the conclusion of NAST, but also of the mechanistic interpretation of the tumor development during NAST. For example, our modeling framework can capture the initial and subsequent responses to A/C as depicted in FIG. 19A-D for patients with very different response dynamics. Third, because the digital twin parameters quantify and elucidate the observed tumor response dynamics, it provides another potential application: predicting response to multiple candidate therapeutic regimens. Thus, digital twins built on quantitative imaging data can provide—early in the course of NAST—a practical way to optimize individual treatment plans and hasten truly personalized cancer care.

Potential variations: First, in various embodiments, instead of utilizing DCE-MRI to inform the spatial distribution of delivered drug, coupling the tumor-response model (i.e., Eq. (T2)) with drug delivery and/or tumor angiogenesis models could estimate drug distribution more accurately. Second, in various embodiments, instead of assuming cell death is merely proportional to drug concentration (i.e., the last term on the right-hand side of Eq. (T2)), a model accounting for detailed therapeutic mechanisms and pharmacodynamics could improve predictive accuracy. Third, the methodology of this Additional Examples section is not currently capable of predicting invasion to the lymph nodes or axilla, which limits the accuracy for predicting pathological response or residual cancer burden (see Supplemental Section 2.3 for additional analysis). Of course, building more comprehensive models necessitates either more assumptions or more measurements, so a careful balance of model complexity and predictive accuracy must be sought.

While the dataset employed in the study of this Additional Examples section is much larger and more homogeneous than our previous preliminary study, there are still a few points about the cohort that could affect the analysis. Framework 1 excluded four patients with no visible tumor at V2. This decision was made so we did not overestimate the accuracy of our predictions as such patients also show no visible tumor at V3, thereby resulting in a 100%-accurate prediction without really testing the modeling. Additionally, the absence of pathological evaluation at the intermediate time point led to the lack of “ground-truth” for Framework 1. Framework 2 excluded nine patients due to enrollment in other trials, causing the cohort to be enriched with pCR patients. This could lead to an overestimation of accuracy for differentiating pCR/non-pCR (see Supplemental Section 2.4 for additional analysis).

The processing (e.g., segmentation, registration) steps are potential sources of error that can be propagated through the modeling pipeline and lead to bias in the prediction. A detailed investigation suggested that un-anticipated changes in ADC values and distribution, as well as the appearance of necrotic regions, are potential sources of error (see Supplemental Section 2.5). Additionally, Framework 1 involved sampling the drug decay rates, which introduces an uncertainty in the predicted tumor response and limits the accuracy for predicting final pathology (see Supplemental Section 2.6). However, compared to previous attempts of simultaneously calibrating the drug efficacy and decay, this procedure not only ensures a more robust model calibration, but also allows the uncertainty in tumor dynamics to be quantified and interpreted. Another source of uncertainty is the setting of drug efficacy and decay rate of paclitaxel in Framework 2, due to the lack of imaging during the paclitaxel portion of NAST. One solution is to incorporate more measurements during NAST, especially after alternating the therapies, so that the digital twin can be updated to preserve an accurate prediction. Of course, it is important to note the goal of the digital twin is not to provide a perfect reproduction of the patient's situation. Rather, a realistic goal is to provide an accurate and practical formalism that provides clinically actionable insights. In the present contribution, we have achieved this goal in the context of predicting the response of early-stage triple-negative breast cancer to neoadjuvant systemic chemotherapy.

This study also supports the value of longitudinal MRI in cancer care. Currently, only pre-treatment MRI is standard for assessing breast cancer patients. While increasing to multiple MRIs would increase the cost for imaging, the benefit of early detection of (for example) chemo-resistance would help to avoid unnecessary toxicity and costs. Both this and prior studies showed that follow-up imaging after the first 1-3 cycles, of NAST is helpful for the early prediction of response. Including even one follow-up MRI (enough to enable model calibration) would be of great benefit.

These embodiments provide patient-specific digital twins via integrating longitudinal, multiparametric MRI data with biologically-based mathematical modeling. This technique accurately captures the spatiotemporal response of TNBC to NAST and achieves high accuracy and specificity for predicting the final pathological status for each individual patient. The success of this approach demonstrates the potential of digital twins to shift the paradigm from assessing response to predicting and, eventually, optimizing response.

TABLE T3
Definition and assignment of the model variables and parameters

| Quantities | | Definition | Assignment |
|---|---|---|---|
| Domains | Ω | Breast tissue domain | Generated from pre-treatment |
| | | | anatomical MRI |
| | T | End time point of | Assigned based on clinical |
| | | NAST procedure | data |
| Variables | x | Spatial coordinate in | Associated with spatial |
| | | breast tissue | domain, Ω |
| | t | Time | Associated with temporal |
| | | | domain, t ∈ [0, T] |
| | N_{I}(x, t) | Image-measured | Computed via Eq. 1 from |
| | | tumor cell number | main text |
| | N(x, t) | Modeled tumor cell | Initialized from pre-treatment |
| | | number | N_{I}(x, 0), computed via Eq. 2 |
| | | | from main text |
| | D(x, t) | Diffusive mobility of | Computed via Eq. 3 from |
| | | tumor cells | main text |
| | λᵢ(x, t) | Death rate induced | Computed via Eq. 4 from |
| | | by the i^{th} drug | main text, i = 1 and 2 for |
| | | | A/C, and i = 3 for paclitaxel |
| | σ(x, t) | von Mises stress | Computed from gradient |
| | | | of N(x, t), based on (1) |
| | Cᵢ(x, •) | Spatiotemporal | Assigned based on DCE-MRI |
| | | distribution of i^{th} | |
| | | drug | |
| | Jᵢ | Total number of | Assigned based on clinical |
| | | administrations of | data |
| | | the i^{th} drug | |
| | τ_{i,j} | Time of the j^{th} | Assigned based on clinical |
| | | administration for | data |
| | | the i^{th} drug | |
| Parameters | k(x) | Proliferation rate of | Locally calibrated^{a} |
| | | tumor cells | |
| | θ | Tumor cells carry | Assigned based on (2) |
| | | capacity | |
| | αᵢ | Efficacy rate of the | Globally calibrated^{b} |
| | | i^{th} drug | |
| | βᵢ | Decay rate of the i^{th} | Globally calibrated or |
| | | drug | sampled |
| | D₀ | Diffusion coefficient | Globally calibrated |
| | | of tumor cells in the | |
| | | absence of mechanical | |
| | | restrictions | |
| | γ | Stress-tumor cell | Assigned based on (1) |
| | | diffusion coupling | |
| | | constant | |

^{a}“Locally calibrated” means the parameter is calibrated to yield a value for each individual voxel in eachh patient’s tumor ROI; i.e., locally calibrated parameter is a spatially varying map.
^{b}“Globally calibrated” means the parameter is calibrated to yied a value for each patient’s whole tumor ROI; i.e., globally calibrated parameter is a scalar.

### Supplemental Section 1: Supplemental Methods

### Supplemental Section 1.1 Determination of Drug Spatial Distribution

As indicated in Eq. (T4), the spatial distribution of i^{th} drug caused by the j^{th} administration of this therapy, C(x, τ_{i,j}) is determined by the enhancement observed via the DCE-MRI measurement (3). Specifically, for each voxel in the breast, the area under the dynamic curve (auc) of the baseline-subtracted DCE time course (i.e., DCE-MRI intensity time course minus the intensity of the pre-contrast frame) is calculated, and this voxel-wise auc is then normalized by dividing the maximum auc value within the tumor. The voxel-wise normalized auc therefore represents the map of peak drug concentration induced by the injection at time τ_{i,j}. This method allows for capturing the spatially non-uniform distribution of each drug as determined by the individual patient's perfusion characteristics.

For each injection, the calculation described in the last paragraph is performed on the last DCE-MRI scan collected before the injection of the drug. Specifically in this study, the spatial distributions of doxorubicin and cyclophosphamide caused by the first and second cycles are calculated from the DCE-MRI data from the V1 scan; the spatial distributions of doxorubicin and cyclophosphamide caused by the third and fourth cycles are calculated from the DCE-MRI data from the V2 scan; the spatial distribution of paclitaxel caused by each cycle is calculated from the DCE-MRI data from the V3 scan. This method allows accounting for changes in the vasculature over the therapeutic regimen.

The above methodology relies on two assumptions: 1) the drug concentration at each location in the domain is approximately proportional to the auc of local DCE-MRI intensity time course, and 2) the washout and efficacy decay of delivered drugs can be approximately represented by voxel-wise exponential decay. We acknowledge that these assumptions, while practical, may not be the most accurate. In future efforts, we desire to couple the tumor-response model (i.e., Eq. 2 from the main text) with more a rigorous description of drug delivery and tumor angiogenesis (4, 5) to achieve a more accurate spatial and temporal representation of drug distribution.

### Supplemental Section 1.2: Concordance Correlation Coefficient

The concordance correlation coefficient (CCC) (6) measures the agreement between two samples (i.e., x and y) via

$$C C C = \frac{2 CoV ( x , y )}{Var ( x ) + Var ( x ) + {( \overline{x} - \overline{y} )}^{2}} , \qquad ( T5 )$$

where x̅ and y̅ are the means of sample x and sample y, respectively; Var(x), Var(y), and CoV(x,y) are the variance of x, variance of y, and the covariance between x and y, respectively.

### Supplemental Section 2: Supplemental Results

### Supplemental Section 2.1: Uncertainty in the Drug Efficacy

As indicated in the discussion of methods in the Additional Examples section, there is uncertainty in both Framework 1 and Framework 2 in the description of drug efficacy and decay.

For Framework 1, the mathematical model is calibrated with the images from V1 and V2. The efficacy of the i^{th} drug and its decay is defined in Eq. (T2) (i.e., αᵢe^{-β}^{ᵢ}^{(t-τ}^{_{i,j}}⁾) in which the efficacy (αᵢ) and decay (βᵢ) rates are strongly coupled. That is, directly fitting αᵢe^{-β}^{ᵢ}^{(t-τ}^{_{i,j}}⁾ to two time points leads to an infinite number of solutions for αᵢ and βᵢ. Therefore, instead of directly fitting/calibrating, we randomly sample each βᵢ within a physical range based on literature values (7) so that for each sampled βᵢ, the corresponding αᵢ can be calibrated. Specifically, for doxorubicin, the decay rate β₁ is sampled five times within [0.01, 0.6] day⁻¹, resulting in five values of β₁ (i.e., β_{1,1}, β_{1,2}, β_{1,3}, β_{1,4}, and β_{1,5}). For cyclophosphamide, β₂ is sampled five times within [1.0, 5.4] day⁻¹, resulting in five values of β₂ (i.e., β_{2,1}, β_{2,2}, β_{2,3}, β_{2,4}, and β_{2,5}). Then, the results of each sample were combined to yield five sampled sets, {β_{1,1}, β_{2,1}}, {β_{1,2}, β_{2,2}}, {β_{1,3}, β_{2,3}}, {β_{1,4}, β_{2,4}}, and {β_{1,5}, β_{2,5}}. With each sampled set, the efficacy rates {α₁, α₂} are calibrated, resulting in five parameter sets of {α₁, α₂, β₁, β₂}. The five parameter sets represent a possible range of A/C efficacy in the individual patient, introducing a quantified source of uncertainty.

Fortunately, based on the results of this study, this approach only leads to a small uncertainty in model outputs of predicted tumor dynamics in Framework 1 (see FIG. 19). Additionally, even with the uncertainty, the model successfully captures substantial differences in tumor dynamics between patients with different responses. As shown in FIGS. 19A and 19B from the main text, the patient with poor response (i.e., 33% shrinkage of tumor volume at the completion of A/C) has much higher tumor cell proliferation rates than the patient with good response (i.e., 100% shrinkage of tumor volume at the completion of A/C): median k(x)=0.05 day⁻¹ versus 0.004 day⁻¹. Additionally, the poor-response patient has a high sensitivity to cyclophosphamide, but low sensitivity to doxorubicin (median α₁=0.24 day⁻¹, α₂=0.60 day⁻¹); in contrast, the good-response patient has a moderate sensitivity to both doxorubicin and cyclophosphamide (median α₁=0.47 day⁻¹, α₂=0.34 day⁻¹). In future work, it is possible that a large sample set of drug decay rates, combined with Bayesian approaches for quantifying the error propagation from model parameters to outcomes, can more completely address the question of how much the uncertainty in these parameters is acceptable before it begins to affect the final model prediction.

For Framework 2, the mathematical model is calibrated with images from V1, V2, and V3. The three-time-point data enables the efficacy rate (αᵢ) and decay rate (βᵢ) to be uniquely calibrated; thus, we chose to calibrate {α₁, α₂, β₁, β₂} simultaneously without the sampling scheme (described in the last paragraph) to avoid the uncertainty in the A/C regimen. However, another uncertainty exists regarding the second regimen, since no data are available to calibrate the decay and efficacy rates of paclitaxel (i.e., α₃ and β₃). To address this situation, the efficacy of paclitaxel is set to literature value; α₃=0.3 day⁻¹ (6). The decay rate of paclitaxel was taken as the average of calibrated A/C decay rates; β₃=(β₁+β₂)/2.

This uncertainty could undermine the potential heterogeneity in patients' sensitivity to the paclitaxel therapy in Framework 2. This limitation arose from the lack of monitoring data which does not allow for update the patient-specific digital twin when the therapeutic regimen changed from A/C to paclitaxel. In general, we lack data types that could help to inform each patients' sensitivity to the paclitaxel. Therefore, future efforts must seek to address this point. One option is to longitudinally monitor (multiple times) the patient's response, especially when the therapies are changed, so that the digital twin can be continuously calibrated and refined to preserve an accurate prediction. The other is to integrate multi-dimensional/multi-type data (e.g., genetic profiles), to inform the assignment of model parameters.

### Supplemental Section 2.2: Interpretation of Uncertainty in the Model Prediction

In Framework 1, the range of model predictions represents the uncertainty in an individual patient's tumor response dynamics under alternative drug pharmacokinetic (i.e., drug decay rates) scenarios. As revealed in FIGS. 19E and 19F, most patients in the cohort have small uncertainty in the predicted tumor response dynamics, while a few patients have relatively large uncertainty.

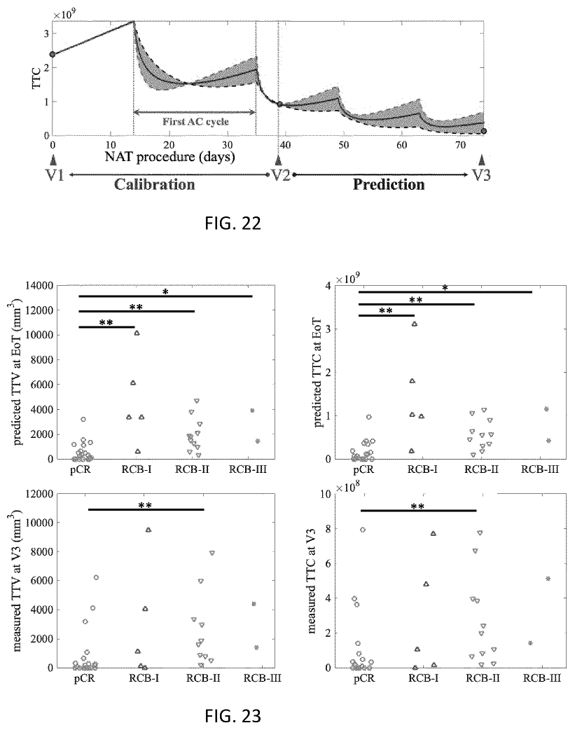

For example, FIG. 22 shows the predicted TTC time courses (median and range) for the patient showing the largest range in FIG. 19E.

Two TTC time courses in the predicted range are highlighted: one from assuming fast drug decays (magenta dash curve; sampled β₁=0.7 day⁻¹ and β₂=5.2 day⁻¹), and the other from assuming slow drug decays (black dash curve; sampled β₁=0.1 day⁻¹ and β₂=1.7 day⁻¹). Within the first A/C cycle (indicated by the green arrow), the trajectory with fast drug decay identifies a strong, rapid response of the tumor followed by a strong regrowth as well; in contrast, the trajectory with small drug decay identifies a relatively slow response with almost no apparent regrowth. Such dramatically different dynamics under the sampled drug pharmacokinetics in this patient lead to a “scissor-shaped” range within the first AC cycle, and further results in a large uncertainty at final prediction of TTC (similarly for TTV). This is very different from the patients we see in FIGS. 19C and 19D, which show “spindle-shaped” ranges within the first AC cycle and have a small uncertainty in the final prediction. Specifically, for the patient in FIG. 19C, all the TTC trajectories predict rapid response and strong regrowth; and for the patient in FIG. 19D, all the TTC trajectories predict weak regrowth.

This observation means, by “looking at” the V1 and V2 data, our model “thinks” there is a large range of possibilities in the underneath tumor dynamics that could lead to the imaging measured tumor change in this specific patient. This is actually an interesting insight, because one promising usage of digital twin techniques is to guide personalized monitoring exams-patients who show large uncertainty in early prediction could be recommended to go with more frequent follow-up imaging (or other) exams, while patients who show very small uncertainty could be recommended for lower frequency follow-ups. In future work, we also would like to further investigate the imaging characteristics, as well as the molecular profiles from intermediate biopsies to gain a better understanding of which features actually result in the large uncertainty of tumor response dynamics in these patients.

### Supplemental Section 2.3: Comparison Between TTV/TTC and Pathological Residual Cancer Burden

In addition to predicting pCR status, we further compared the model predictions and imaging measurements to the pathological residual cancer burden (RCB). Specifically, RCB was estimated from routine pathologic sections of the primary breast tumor site and the regional lymph nodes after the completion of neoadjuvant therapy. Among the 37 patients included in Framework 2, 19 patients are pCR, 5 patients are RCB-I, 11 patients are RCB-II, and 2 patients are RCB-III. The RCB-I, -II, -III classes are considered as the non-pCR group.

FIG. 23 presents the predicted TTV at the end of treatment (EoT), the predicted TTC at EoT, the measured TTV at V3, and the measured TTC at V3, respectively, versus the RCB-classes. For each metric investigated, we also performed the Wilcoxon test between each pair of RCB-classes (* indicates P<0.05 and ** indicates P<0.01 in FIG. 23).

For the predicted TTV at EoT, significant differences were found between the pCR group and each non-pCR group; with P=0.003 for pCR vs RCB-I, P=0.001 for pCR vs RCB-II, and P=0.046 for pCR vs RCB-III, respectively. Similarly, for the predicted TTC at EoT, significant differences were found between the pCR group and each non-pCR group; with P=0.003 for pCR vs RCB-I, P=0.001 for pCR vs RCB-II, and P=0.034 for pCR vs RCB-III, respectively. However, no significant difference was found for predicted TTV or TTC between either pair of non-pCR groups. In contrast, for the measured TTV at V3, a significant difference was only found between the pCR and RCB-II groups, with P=0.006. Similarly, for the measured TTC at V3, a significant difference was only found between the pCR and RCB-II groups, with P=0.006. No significant difference was found for measured TTV or TTC between the other pairs of RCB classes.

These results support the position that the digital twin-based prediction improves the ability to discriminate pCR and non-pCR. However, neither the predictions up to the end of NAST or the imaging measurements at the end of A/C can provide a good discrimination between different non-pCR RCB classes. This is possibly due to the fact that neither the imaging measurements nor the prediction scheme in the Additional Examples section considered invasion into the lymph nodes.

### Supplemental Section 2.4: Stratified Analysis in the 9 Patients Excluded From Framework 2

Considering nine patients were excluded from Framework 2 due to enrollment in other clinical trials, we performed a stratified analysis in the nine-patient subset to assess the potential impact of this exclusion on the evaluation.

We repeated the Framework 1 evaluation in this nine-patient subset. The CCC between the measured and predicted change of TTC is 0.83, the CCC between the measured and predicted change of TTV is 0.79, and the difference between the measured and predicted change of tumor cell distributions has a mean (95% CI) of −1.33% (−41.83%-39.18%). Thus, the prediction accuracy of Framework 1 in this 9-patient subset is lower than that in the whole cohort (shown in the Table T1). This observation suggests that excluding these nine patients from Framework 2 may lead to an overestimation of accuracy for differentiating pCR versus non-pCR.

### Supplemental Section 2.5: Investigation on Patients With Suboptimal Predictive Accuracy

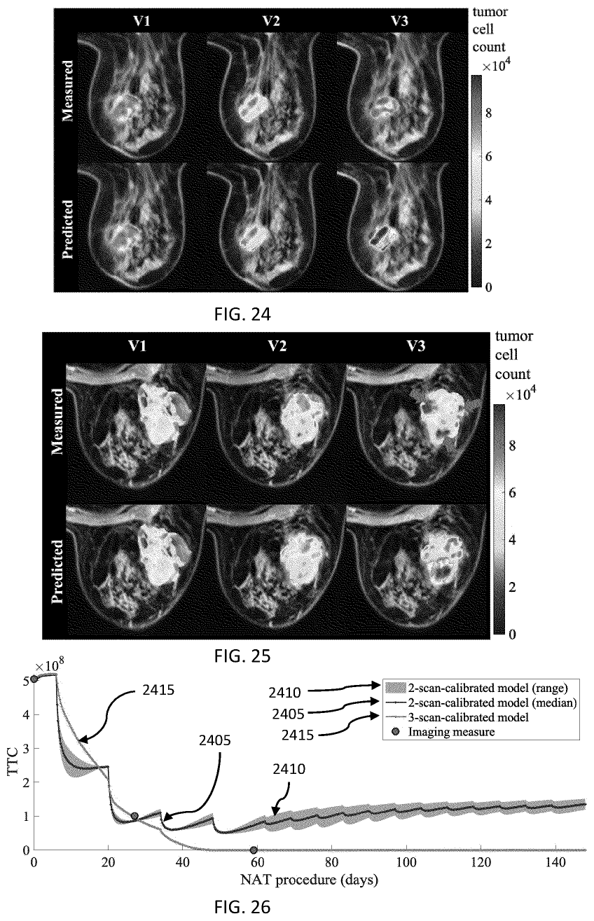

As shown in FIG. 20E, four patients (i.e., Patients 5, 11, 26, and 49) were identified to have suboptimal predictive accuracy (i.e., predictions were outside the mean 95% CI of the cohort). Careful investigation indicated that the suboptimal predictions in these patients are related to unexpected changes in the spatial pattern of measured tumor response across the treatment. For example, the panels in FIG. 24 show the measured and predicted tumor cell distributions on the central tumor slice for Patient 26 in FIG. 20E.

Considering the top row, we see the tumor cells distribute almost uniformly at the V1 measurement, tend to accumulate to the medial-posterior and lateral-anterior of the tumor at V2, and change to accumulate along the periphery of the tumor at V3. Based on the imaging measurements at V1 and V2, the change of cell distribution from V2 to V3 is quite unexpected. Thus, after calibrating with V1 and V2, the model predicted the tumor response with a spatial pattern of high proliferation around the medial-posterior and lateral-anterior regions, and high death in other regions, which leads to a big difference as compared to the measured V3. Such change in the spatial pattern of tumor response across longitudinal imaging could be due to the evolution of the tumor microenvironment and clonal population, which cause tumors to respond very differently between the early and late phases of AC therapy. Alternatively, it could be due to error in the measured ADC. In future work, we would like to further investigate the image quality, the imaging characteristics, as well as the molecular profiles from intermediate biopsies of these patients to gain better an understanding of tumor response over NAST.

Another factor that could lead to the unexpected change in the spatial pattern is the presence of large necrotic regions. For example, the panels of FIG. 25 show the measured and predicted tumor cell distributions on the central tumor slice for Patient 49 in FIG. 20E. The appearance of large necrotic regions in V3 causes difficulty in predicting the final tumor shape.

### Supplemental Section 2.6: 2-MRI Calibration Versus 3-MRI Calibration

The investigation indicated both improved accuracy and reduced uncertainty in prediction with an additional MR scan used for calibration. Consider the patient in the FIG. 21C as an example. FIG. 26 plots the whole-NAST prediction with the 2-scan-calibrated model (2405 and 2410), and the 3-scan-calibrated model (2415). The results indicate that the 2-scan-calibrated model successfully predicts an early and strong response in the patient, but fails to predict the complete shrinkage of the tumor. In contrast, including the third MRI scan in the calibration enables the model to fully capture the shrinkage of the tumor, leading to a successful prediction of pCR for this patient.

Non-limiting example embodiments are provided here:

Embodiment A: A method comprising: acquiring, by a computing system, magnetic resonance imaging (MRI) data corresponding to a plurality of MRI scans of an anatomical region comprising a tumor of a patient, the plurality of MRI scans comprising a first set of images obtained through a first scan performed prior to an administration of a therapy to the patient and a second set of images obtained through a second scan performed following the administration of the therapy to the patient, the therapy including administration of a plurality of drugs; determining, by the computing system, from MRI data, tissue properties of tissue surrounding the tumor; registering, by the computing system, image-related data generated from the second set of images with image-related data generated from the first set of images; determining, by the computing system, diffusion characteristics of the tumor based on the tissue properties; determining, by the computing system, growth characteristics of the tumor based on the tissue properties; determining, by the computing system, for each drug of the plurality of drugs, an effect of the drug on tumor cells; and generating, by the computing system, based on the diffusion characteristics of the tumor, the growth characteristics of the tumor, and the determined effect of each drug on cells included in each voxel, a score indicating a predicted response of the tumor to the therapy.

Embodiment B: The method of Embodiment A, further comprising performing tumor segmentation to identify a tumor region of interest (ROI) based on the MRI data prior to determining the tissue properties.

Embodiment C: The method of Embodiment A or B, wherein the tissue properties are related to vasculature in the tissue.

Embodiment D: The method of any of Embodiments A-C, wherein the MRI data comprises dynamic contrast enhanced MRI (DCE-MRI) data, and wherein the tissue properties are quantified based on the DCE-MRI data.

Embodiment E: The method of any of Embodiments A-D, wherein the tissue properties are quantified based on a pharmacokinetic model.

Embodiment F: The method of any of Embodiments A-E, wherein the tissue properties are quantified based on a fluid mechanics model.

Embodiment G: The method of any of Embodiments A-F, wherein the tissue properties are quantified based on a Kety-Tofts model or a variation of the Kety-Tofts model.

Embodiment H: The method of any of Embodiments A-G, wherein the MRI data comprises diffusion-weighted MRI (DW-MRI), the method further comprising generating a map of apparent diffusion coefficient (ADC) of water.

Embodiment I: The method of any of Embodiments A-H, wherein registering the image-related data from the second set of images with the image-related data generated from the first set of images aligns images with the map into a common domain.

Embodiment J: The method of any of Embodiments A-I, further comprising determining drug distribution in each voxel of tissue.

Embodiment K: The method of any of Embodiments A-J, wherein determining drug distribution in each voxel of tissue comprises generating a normalized map of blood volume to define initial drug distribution throughout the domain at time of each dose of therapy.

Embodiment L: The method of any of Embodiments A-K, wherein the diffusion characteristics correspond to diffusion of the tumor cells as mechanically linked to material properties of the tissue via a physical stressor, thereby representing tumor changes that can cause deformations in the tissue.

Embodiment M: The method of any of Embodiments A-L, wherein the growth characteristics are based on a carrying capacity related to a maximum number of tumor cells that can physically fit within a voxel.

Embodiment N: The method of any of Embodiments A-M, wherein the growth characteristics are based on a proliferation rate per voxel, the proliferation rate calibrated per voxel within the tumor ROI for the patient.

Embodiment O: The method of any of Embodiments A-N, wherein the effect of each drug on tumor cells is based on concentration of the drug in the tissue.

Embodiment P: The method of any of Embodiments A-O, wherein the effect of each drug on tumor cells corresponds to a spatiotemporal distribution of each drug in the tissue.

Embodiment Q: The method of any of Embodiments A-P, wherein the effect of each drug on tumor cells is based on at least one of an efficacy parameter α of the drug, a washout parameter β of the drug over time after each dose, or an initial concentration of the drug.

Embodiment R: The method of any of Embodiments A-Q, wherein the effect of each drug on tumor cells is based on an efficacy parameter α of the drug, a washout parameter β of the drug over time after each dose, and an initial concentration of the drug.

Embodiment S: The method of any of Embodiments A-R, wherein at least one of the efficacy parameter and the washout parameter is calibrated for the patient or the drug.

Embodiment T: The method of any of Embodiments A-S, wherein calibration of the washout parameter for the patient is restricted using bounds defined from ranges for terminal elimination half-lives of the drug.

Embodiment U: The method of any of Embodiments A-T, wherein the MRI data comprises dynamic contrast enhanced MRI (DCE-MRI) data, and wherein the initial concentration is approximated using the DCE-MRI data.

Embodiment V: The method of any of Embodiments A-U further comprising performing rigid or nonrigid intrascan registration of MRI data in each of the first set of images and the second set of images.

Embodiment W: The method of any of Embodiments A-V, further comprising determining a modified therapy based on the score indicating the predicted response of the tumor to the therapy.

Embodiment X: The method of any of Embodiments A-W, further comprising administering a modified therapy based on the score indicating the predicted response of the tumor to the therapy.

Embodiment Y: The method of any of Embodiments A-X, wherein the therapy and/or the modified therapy is a neoadjuvant therapy (NAT).

Embodiment AA: A computing system comprising one or more processors and a computer-readable memory with instructions configured to cause the one or more processors to: acquire MRI data corresponding to a plurality of MRI scans of an anatomical region comprising a tumor, the plurality of MRI scans comprising a first set of images obtained through a first scan performed prior to an administration of a therapy to a patient and a second set of images obtained through a second scan performed following the administration of the therapy to the patient, the therapy including administration of a plurality of drugs; determine from MRI data, one or more tissue properties of tissue surrounding the tumor; register image-related data generated from the second set of images with image-related data generated from the first set of images; determining diffusion characteristics of the tumor based on the tissue properties; determining growth characteristics of the tumor based on the tissue properties; determining, for each drug of the plurality of drugs, an effect of the drug on tumor cells; and generate, based on the diffusion characteristics of the tumor, the growth characteristics of the tumor, and the determined effect of each drug on cells included in each voxel, a score indicating a predicted response of the tumor to the therapy.

Embodiment BB: The system of Embodiment AA, the instructions further configured to cause the one or more processors to perform tumor segmentation to identify a tumor ROI based on the MRI data prior to determining the tissue properties.

Embodiment CC: The system of Embodiment AA or BB, wherein the tissue properties are related to vasculature in the tissue.

Embodiment DD: The system of any of Embodiments AA-CC, wherein the MRI data comprises dynamic contrast enhanced MRI (DCE-MRI) data, and wherein the tissue properties are quantified based on the DCE-MRI data.

Embodiment EE: The system of any of Embodiments AA-DD, wherein the tissue properties are quantified based on a pharmacokinetic model, a fluid mechanics model, a Kety-Tofts model, and/or a variation of the Kety-Tofts model.

Embodiment FF: The system of any of Embodiments AA-EE, wherein the MRI data comprises diffusion-weighted MRI (DW-MRI), the instructions further configured to cause one or more processors to generate a map of apparent diffusion coefficient (ADC) of water.

Embodiment GG: The system of any of Embodiments AA-FF, wherein registering the image-related data from the second set of images with the image-related data generated from the first set of images aligns images with the map into a common domain.

Embodiment HH: The system of any of Embodiments AA-GG, the instructions further configured to cause one or more processors to estimate drug distribution in each voxel of tissue.

Embodiment II: The system of any of Embodiments AA-HH, wherein determining drug distribution in each voxel of tissue comprises generating a normalized map of blood volume to define initial drug distribution throughout the domain at time of each dose of therapy.

Embodiment JJ: The system of any of Embodiments AA-II, wherein the diffusion characteristics correspond to diffusion of the tumor cells as mechanically linked to material properties of the tissue via a physical stressor, thereby representing tumor changes that can cause deformations in the tissue.

Embodiment KK: The system of any of Embodiments AA-JJ, wherein the growth characteristics are based on a carrying capacity related to a maximum number of tumor cells that can physically fit within a voxel.

Embodiment LL: The system of any of Embodiments AA-KK, wherein the growth characteristics are based on a proliferation rate per voxel, the proliferation rate calibrated per voxel within the tumor ROI for the patient.

Embodiment MM: The system of any of Embodiments AA-LL, wherein the effect of each drug on tumor cells is based on concentration of the drug in the tissue.

Embodiment NN: The system of any of Embodiments AA-MM, wherein the effect of each drug on tumor cells corresponds to a spatiotemporal distribution of each drug in the tissue.

Embodiment OO: The system of any of Embodiments AA-NN, wherein the effect of each drug on tumor cells is based on at least one of an efficacy parameter α of the drug, a washout parameter β of the drug over time after each dose, or an initial concentration of the drug.

Embodiment PP: The system of any of Embodiments AA-OO, wherein the effect of each drug on tumor cells is based on an efficacy parameter α of the drug, a washout parameter β of the drug over time after each dose, and an initial concentration of the drug.

Embodiment QQ: The system of any of Embodiments AA-PP, wherein at least one of the efficacy parameter and the washout parameter is calibrated for the patient or the drug.

Embodiment RR: The system of any of Embodiments AA-QQ, wherein calibration of the washout parameter for the patient is restricted using bounds defined from ranges for terminal elimination half-lives of the drug.

Embodiment SS: The system of any of Embodiments AA-RR, wherein the MRI data comprises DCE-MRI data, and wherein the initial concentration is approximated using the DCE-MRI data.

Embodiment TT: The system of any of Embodiments AA-SS, the instructions further configured to cause the one or more processors to perform rigid and/or nonrigid intrascan registration of MRI data in each of the first set of images and the second set of images.

Embodiment UU: The system of any of Embodiments AA-TT, the instructions further configured to cause the one or more processors to determine a modified therapy based on the score indicating the predicted response of the tumor to the therapy.

Embodiment VV: The system of any of Embodiments AA-UU, wherein the therapy and/or the modified therapy is a neoadjuvant therapy (NAT).

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that the terms “exemplary,” “example,” “potential,” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the Figures. It should be noted that the orientation of various elements may differ according to other exemplary embodiments, and that such variations are intended to be encompassed by the present disclosure.

The embodiments described herein have been described with reference to drawings. The drawings illustrate certain details of specific embodiments that implement the systems, methods and programs described herein. However, describing the embodiments with drawings should not be construed as imposing on the disclosure any limitations that may be present in the drawings.

It is important to note that the construction and arrangement of the devices, assemblies, and steps as shown in the various exemplary embodiments is illustrative only. Additionally, any element disclosed in one embodiment may be incorporated or utilized with any other embodiment disclosed herein. Although only one example of an element from one embodiment that can be incorporated or utilized in another embodiment has been described above, it should be appreciated that other elements of the various embodiments may be incorporated or utilized with any of the other embodiments disclosed herein.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described in order to explain the principals of the disclosure and its practical application to enable one skilled in the art to utilize the various embodiments and with various modifications as are suited to the particular use contemplated. Other substitutions, modifications, changes and omissions may be made in the design, operating conditions and arrangement of the embodiments without departing from the scope of the present disclosure as expressed in the appended claims.

What is claimed is:

1. A method comprising:
acquiring, by a computing system, magnetic resonance imaging (MRI) data corresponding to a plurality of MRI scans of an anatomical region comprising a tumor of a patient, the plurality of MRI scans comprising a first set of images obtained through a first scan performed prior to an administration of a therapy to the patient and a second set of images obtained through a second scan performed following the administration of the therapy to the patient, the therapy including administration of a plurality of drugs;
determining, by the computing system, from MRI data, tissue properties of tissue surrounding the tumor;
registering, by the computing system, image-related data generated from the second set of images with image-related data generated from the first set of images;
determining, by the computing system, diffusion characteristics of the tumor based on the tissue properties;
determining, by the computing system, growth characteristics of the tumor based on the tissue properties;
determining, by the computing system, for each drug of the plurality of drugs, an effect of the drug on tumor cells; and
generating, by the computing system, based on the diffusion characteristics of the tumor, the growth characteristics of the tumor, and the determined effect of each drug on cells included in each voxel, a score indicating a predicted response of the tumor to the therapy.

2. The method of claim 1, further comprising performing tumor segmentation to identify a tumor region of interest (ROI) based on the MRI data prior to determining the tissue properties.

3. The method of claim 1, wherein the tissue properties are related to vasculature in the tissue.

4. The method of claim 1, wherein the MRI data comprises dynamic contrast enhanced MRI (DCE-MRI) data, and wherein the tissue properties are quantified based on the DCE-MRI data.

5. The method of claim 1, wherein the MRI data comprises diffusion-weighted MRI (DW-MRI), the method further comprising generating a map of apparent diffusion coefficient (ADC) of water.

6. The method of claim 1, further comprising determining drug distribution in each voxel of tissue.

7. The method of claim 1, wherein the diffusion characteristics correspond to diffusion of the tumor cells as mechanically linked to material properties of the tissue via a physical stressor, thereby representing tumor changes that can cause deformations in the tissue.

8. The method of claim 1, wherein the growth characteristics are based on a carrying capacity related to a maximum number of tumor cells that can physically fit within a voxel.

9. The method of claim 1, wherein the growth characteristics are based on a proliferation rate per voxel, the proliferation rate calibrated per voxel within the tumor ROI for the patient.

10. The method of claim 1, wherein the effect of each drug on tumor cells corresponds to a spatiotemporal distribution of each drug in the tissue.

11. The method of claim 1, wherein the effect of each drug on tumor cells is based on at least one of an efficacy parameter α of the drug, a washout parameter β of the drug over time after each dose, or an initial concentration of the drug.

12. The method of claim 1, further comprising determining a modified therapy based on the score indicating the predicted response of the tumor to the therapy.

13. The method of claim 1, further comprising administering a modified therapy based on the score indicating the predicted response of the tumor to the therapy.

14. A computing system comprising one or more processors and a computer-readable memory with instructions configured to cause the one or more processors to:
acquire MRI data corresponding to a plurality of MRI scans of an anatomical region comprising a tumor, the plurality of MRI scans comprising a first set of images obtained through a first scan performed prior to an administration of a therapy to a patient and a second set of images obtained through a second scan performed following the administration of the therapy to the patient, the therapy including administration of a plurality of drugs;
determine from MRI data, one or more tissue properties of tissue surrounding the tumor;
register image-related data generated from the second set of images with image-related data generated from the first set of images;
determining diffusion characteristics of the tumor based on the tissue properties;
determining growth characteristics of the tumor based on the tissue properties;
determining, for each drug of the plurality of drugs, an effect of the drug on tumor cells; and
generate, based on the diffusion characteristics of the tumor, the growth characteristics of the tumor, and the determined effect of each drug on cells included in each voxel, a score indicating a predicted response of the tumor to the therapy.

15. The computing system of claim 14, the instructions further configured to cause the one or more processors to perform tumor segmentation to identify a tumor ROI based on the MRI data prior to determining the tissue properties.

16. The computing system of claim 14, wherein the tissue properties are related to vasculature in the tissue.

17. The computing system of claim 26, wherein the MRI data comprises diffusion-weighted MRI (DW-MRI), the instructions further configured to cause one or more processors to generate a map of apparent diffusion coefficient (ADC) of water.

18. The computing system of claim 14, wherein the growth characteristics are based on a carrying capacity related to a maximum number of tumor cells that can physically fit within a voxel.

19. The computing system of claim 14, wherein the effect of each drug on tumor cells is based on at least one of an efficacy parameter α of the drug, a washout parameter β of the drug over time after each dose, or an initial concentration of the drug.

20. The computing system of claim 14, further comprising determining a modified therapy based on the score indicating the predicted response of the tumor to the therapy.
